(19) United States
(12) Patent Application Publication
(10) Pub. No.: US 2009/0197336 A1
(43) Pub. Date: Aug. 6, 2009

(54) SUPPRESSION OF POLYMORPHIC ALLELES

(76) Inventors: Gwenyth Jane Farrar, Monkstown (IE); Peter Humphries, Cabinteeley D18 (IE); Sophia Millington-Ward, Glasnevin (IE); Paul Francis Kenna, Dublin (IE)

Correspondence Address: WOLF GREENFIELD & SACKS, P.C., 600 ATLANTIC AVENUE, BOSTON, MA, 02210-2206, US

(21) Appl. No.: 12/188,405

(22) Filed: Aug. 8, 2008

Related U.S. Application Data
(63) Continuation of application No. 10/000,773, filed on Nov. 30, 2001, now abandoned.
Application No. 10/000,773 is a continuation-in-part of application No. 09/142,125, filed on Apr. 12, 1999, now abandoned.

(30) Foreign Application Priority Data
Mar. 1, 1996 (GB) 9604449.0
Mar. 3, 1997 (GB) PCT/GB97/00574

(51) Int. Cl. C12N 15/85; C12N 9/22

(52) U.S. Cl. 435/455; 435/199

(57) ABSTRACT

Methods and agents for suppressing expression of a mutant allele of a gene having a polymorphism are provided. The methods of the invention provide suppression effectors such as antisense nucleic acids or ribozymes, that bind to nucleic acid regions having a polymorphism within a gene such that one allele of a gene is exclusively or preferentially suppressed. The method also provides the administration of a replacement nucleic acid, if required. The invention has the advantage that the same suppression strategy, when directed to polymorphisms, could be used to suppress, in principle, many mutations in a polymorphic gene. This is particularly relevant when large numbers of mutations within a single gene cause disease pathology.

### RELATED APPLICATIONS

This application is a continuation-in-part application of U.S. Ser. No. 09/142,125, filed Apr. 12, 1999, which claims priority to PCT/GB97/00574, filed Mar. 3, 1997; which claims priority to GB 9604449.0, filed Mar. 1, 1996; the entire disclosures of which are hereby incorporated by reference.

### FIELD OF THE INVENTION

the invention relates to methods and reagents for suppressing a gene which has a polymorphism.

### BACKGROUND OF THE INVENTION

Polymorphisms are present in the coding and non-coding sequences of most genes. Polymorphisms such as, for example, simple sequence repeats, insertions, deletions or single nucleotide changes (either silent changes or changes resulting in amino acid substitutions) have been observed in many normal and disease human genes. Further, as the human genome sequencing project proceeds, the level of polymorphism in the genome will be more accurately defined and increasing numbers of intragenic polymorphisms will become known.

The polymorphic nature of the human genome, and that of many other species, provides a means for mapping genetic disease without necessarily knowing the identity of the gene or genes involved. For example, retinitis pigmentosa (RP) is a hereditary degenerative ocular disorder which is genetically heterogeneous in nature and characterized by genetic polymorphisms. Many different mutations in various RP genes have been identified, characterized and mapped. For example, applying the approach of genetic linkage, x-linked RP (x1 RP) genes have been localised to the short arm of the X chromosome (Ott et al., 1990). The gene involved in one form of x1 RP was subsequently identified. In addition, various genes involved in autosomal dominant forms of RP (adRP) have been localized. The first of these mapped to 3q, close to the gene encoding the rod photoreceptor protein rhodopsin (McWilliam et al., 1989; Dryja et al., 1990). Similarly, an adRP gene was mapped to 6p close to the gene encoding the photoreceptor protein peripherin (Farrar et al., 1991a,b; Kajiwara et al., 1991). Other adRP genes have been mapped to discrete chromosomal locations, but as yet remain uncharacterized. In addition, various genes involved in autosomal recessive RP (arRP) and other macular dystrophies have been localized and, in some cases, have been characterized (Humphries et al., 1992; Farrar et al., 1993; Van Soest et al., 1994; Mansergh et al., 1995).

Genetic linkage and mutational screening techniques have also enabled identification of causative dominant mutations in the genes encoding rhodopsin and peripherin. Globally, about 100 rhodopsin mutations have been found in patients with RP or congenital stationary night blindness. Approximately 40 mutations have been characterized in the peripherin gene in patients with RP or macular dystrophies. Knowledge of the molecular etiology of these retinopathies has lead to the generation of animal models of these diseases and the search for methods of therapeutic intervention (Farrar et al., 1995; Humphries et al., 1997).

In addition, Osteogenesis Imperfecta (OI), often referred to as “brittle bone disease” is caused by one or more mutational events that are characterized by genetic polymorphisms. OI is an autosomal dominant inherited disorder which is caused by mutations in two genes, COL1A1 and COL1A2. Both genes encode the two type 1 pro-collagen proteins, precursor forms of collagen, the most abundant protein in man and the major protein lending strength to bone and fibrous tissue. The disease phenotypes of OI patients vary substantially and have generally been divided into four major categories (Type I, Type II, Type III and Type IV) depending on the specific mutation involved. Type 1 OI, the mildest form of the disease, is characterized by blue sclera, brittle bones, sometimes loss of hearing and dentinogenesis imperfecta and is usually caused by null mutations in the COL1A1 or COL1A2 genes that result in lower levels of protein being formed. Type II OI results in extensive fractures and deformities causing perinatal death. Types III and IV are fairly similar disorders. They are both progressive and cause short stature, hearing loss, fracturing, blue sclera and dentinogenesis imperfecta (Sillence et al., 1979; Byers, 1989, 1990, 1993; Wenstrup et al., 1990).

Type I collagen molecules, consisting of two α1 chains and one α2 chain, are products of two genes, COL1A1 and COL1A2, located on human chromosomes 17 and 7, respectively. Each gene contains more than 50 exons. Mutations in COL1A1 and COL1A2 are known not only to cause OI, but also the autosomal dominant type VII Ehlers-Danlos syndrome (EDS) (Lehmann et al, 1994), a disease which causes loose-jointedness and fragile, bruisable skin that heals with peculiar ‘cigarette-paper’ scars (Barabas, 1966; Byers et al., 1997). Mutations in these genes have also been implicated in Marfan syndrome (Dalgleish et al, 1986; Philips et al., 1990; D'Alessio, et al.; Vasan, N S et al., 1991).

Multiple genes and/or multiple mutations within a gene can give rise to a similar disease phenotype in a number of diseases, including OI, familial hypercholesteremia and RP. For example, there are over 150 different mutations in COL1A1 known to cause OI and over 80 in COL1A2 known to cause either EDS or OI (for an online list see http://www.le.ac.uk/genetics/collagen/col1A1.html). In addition, certain mutations in the COL1A1 gene have been implicated in osteoporosis (Hampson et al., 1998; Keen et al, 1999). These mutations are dominant and many are single point mutations, although a number of insertion and deletion mutations have been found (Willing et al., 1993; Zhuang et al., 1996). Given this heterogeneity there is a need for mutation independent therapeutic agents to treat heterogeneous diseases such as OI (i.e., therapeutics that do not target only a single mutation in a gene).

The treatment of dominantly inherited diseases in many cases requires suppression of the primary mutation or modulation of the dominant effect of the mutation by altering secondary effects associated with the disease pathology, e.g., modulating apoptosis or programmed cell death (Chang et al., 1993; Davidson and Stellar, 1998). Similar treatment strategies can be applied to polygenic disorders involving co-inheritance of a number of genetic components that together give rise to a disease phenotype. Effective gene therapy for monogenic or polygenic diseases caused by dominant negative mutations requires suppression of the disease allele while in many cases still maintaining the function of the normal allele and/or modulating a secondary effect associated with the disease pathology. However, the genetic heterogeneity inherent in many genetic disorders complicates the development of methods of treatment.

Gene therapies utilizing viral and non-viral delivery systems have been used to treat inherited disorders, cancers and infectious diseases. However, many studies have focused on recessively inherited disorders, the rationale being that introduction and expression of the wild-type gene may be sufficient to prevent/ameliorate the disease phenotype. In contrast, gene therapy for dominant disorders (caused by dominant negative mutations) requires suppression of the dominant disease allele. Indeed there are over 1,000 autosomal dominantly inherited disorders in man. A further difficulty is the heterogeneous nature of some dominant disorders, e.g., when many different mutations in the same gene give rise to a similar disease phenotype. Development of specific gene therapies for each of these mutations may be prohibitive in terms of cost and effectiveness. In addition, there are many polygenic disorders due to co-inheritance of a number of genetic components which together give rise to a disease state. Strategies for differentiating between normal and disease alleles and for selectively switching off the disease allele using, for example, antisense DNA/RNA, peptide nucleic acids (PNAs), or triple helix forming oligonucleotides that target a particular mutation are problematic where the disease and normal alleles differ by only a single nucleotide. There is therefore a need for effective gene therapies for dominant or polygenic diseases that target the primary defect, thereby suppressing the disease allele, and in many cases also maintaining the function of a normal allele. In addition, there is a need for suppression therapies that target secondary effects associated with the disease pathology such as, for example, programmed cell death (apoptosis), which has been observed in many inherited disorders.

### SUMMARY OF THE INVENTION

The present invention solves the problem of suppressing dominant negative disease genes using by targeting suppressor effectors to polymorphisms in genes. The invention exploits the utility of polymorphism to discriminate between alleles that carry a mutation which may be independent of the polymorphism and ones that do not. This provides flexibility in the choice of target sequence for suppression, in contrast to suppression strategies directed towards single disease mutations, since many genes have multiple polymorphic target sites.

In one aspect, the invention provides a suppression effector for suppressing endogenous gene expression, the suppression effector binds to a polymorphic site on a DNA or RNA of a target allele of an endogenous gene, thereby suppressing the functional expression of the target allele. The suppression effector is preferably a ribozyme which comprises a nucleic acid sequence that binds to at least 3 to about 100 nucleotides on either side of a polymorphic site. Preferably, the suppression effector suppresses functional gene expression of a target allele and does not suppress functional gene expression of any wild-type allele. In a preferred embodiment, the suppression effector binds to the DNA or RNA of the target allele specifically. In another preferred embodiment, the functional gene expression of the target allele is exclusively suppressed. In yet another preferred embodiment, the functional gene expression of the target allele is preferentially suppressed.

The suppression effector broadly may be an antisense nucleic acid, peptide nucleic acid (PNA), peptide, antibody, and modified forms thereof. In a preferred embodiment, the suppression effector is encoded by a DNA that is incorporated into an expression vector that is operatively linked to nucleic acids which regulate its expression in a host cell. In a particularly preferred embodiment, the suppression effector is a ribozyme, such as a hammerhead ribozyme. In another preferred embodiment, the suppressor effector is a DNA which forms triple helix DNA with the target allele of the gene.

In an embodiment of the invention, the polymorphism or multiple polymorphisms which are the target of the suppressor effectors of the invention is located in one or more sites consisting of coding regions, the 5′ untranslated region, the 3′ untranslated region, intronic regions, control regions, or regions adjacent to a gene to be suppressed. In an embodiment, the control region is a transcription control region or a translation control region.

In one embodiment, the suppression effector increases the effectiveness or action of a compound with which it is co-administered, e.g., by altering drug response. For example, one allelic variant of a drug metabolising enzyme may either metabolise an administered drug too rapidly thereby limiting the bioavailability of the drug and therefore its efficacy or alternatively some allelic variants may metabolise an administered drug too slowly therefore leading to potential toxicity—i.e., too high levels of drug. Hence, it may be that the co-administration of the drug together with suppressing one allelic variant of a gene (e.g., a drug metabolizing enzyme or DME) and replacing it with an alternative allelic variant of the DME would aid in optimizing the effectiveness of the co-administered drug and limit the associated toxicity. In another embodiment, the invention can be used to suppress and replace genes and gene products involved in either the absorption and transport of the drug and/or the receptor target for the drug itself. Some key categories of genes include those encoding drug metabolising enzymes (for example, the cytochrome P450 genes, thiopurine methyl transferase amongst others), genes encoding receptor(s) for drugs (for example, dopamine receptors, β2-adrenergic receptor amongst others) and genes encoding products which alter drug absorption and transport (for example, P-glycoprotein amongst others). For example, the multi-drug resistance gene (MDR-1) encoding P glycoprotein, a member of the ABC transporter family, can significantly influence the bioavailability of chemotherapeutic drugs and over-expression of this gene is responsible for tumour resistance to chemotherapeutics in some cases (Refs: Gottesman M M, Pastan I. Annu Rev Biochem 62: 385-427 1993). Similarly, well over 100 drugs are substrates for one of the cytochrome P450 genes (CYP2D6); various allelic variants have been defined in this gene that can result in significantly altered activity of the encoded protein, for example, allele CYP2D6*5 carries a deletion and hence encodes no enzyme (Skoda R C et al. 1988 PNAS USA 85: 5240-5243; Daly A K et al. 1996 Pharmacogenetics 6: 193-201). Furthermore, studies with the β2-adrenergic receptor gene suggest that a single polymorphic variant at codon 16 (Gly/Arg) of the receptor gene significantly alters response (approximately a 5-fold difference) to bronchodilators such as albuterol (Martinez F D et al. 1997 J Clin Invest 150: 3184-3188). The suppression and replacement of genes involved in altering drug response may aid in optimizing the utility of a broad range of drugs.

In another embodiment, the suppression effector and replacement technology can be used to render a cell or individual which is genetically predisposed to infection by an infectious agent resistant to infection. Given knowledge of the molecular mechanisms of infection and resistance to infectious agents the suppression and replacement technologies described herein can be used in the treatment of infectious agents/disorders.

In another aspect, the invention provides methods for inhibiting the expression of a target allele by providing one or more suppression effectors that bind to a polymorphism within or adjacent a DNA or RNA of a target allele such that the functional expression of one allele of a gene is exclusively or preferentially suppressed. The methods of the invention may be useful for treating any disease, but are particularly useful for treating or preventing diseases that are caused by dominant negative mutations (either monogenic or polygenic).

In another preferred embodiment, the methods of the invention further comprise administering a replacement nucleic acid which replaces the polymorphic allele with a functional wild-type allele or other polymorphic allele not associated with the disease that is associated with the target allele. The replacement nucleic acid may be an artificially derived allelic variant of a target allele, whereby the allelic variant has a different nucleic acid sequence from that of the target allele. Preferably, the suppression effector does not bind, or binds less effectively, to the replacement nucleic acid than to an RNA or DNA encoding the target allele.

The replacement nucleic acid for use in the methods of the invention preferably has a nucleic acid sequence that is different from that of the target allele. The replacement nucleic acid preferably encodes a normal gene product which has equivalent or improved activity compared to the endogenous gene product. Thus, in another aspect, the invention provides replacement nucleic acids, which may be in the form of DNA or RNA sequences that include sequences that allow expression of the replacement nucleic acid in vivo.

In another aspect, the invention provides a treatment for a disease by providing (a) one or more nucleic acids encoding a suppression effector that binds to a DNA or RNA encoding one or more polymorphic sites within or adjacent to a target allele of an endogenous gene, and (b) one or more nucleic acids encoding a replacement nucleic acid to which the suppression effector does not bind, or binds less efficiently than the suppression effector binds to the target allele.

In another aspect, the invention provides a kit comprising a therapeutic for the treatment of a genetic disease caused by a deleterious mutation in one or more alleles of a gene, containing one or more suppression effectors that bind to a DNA or RNA at a polymorphic site within or adjacent a target allele of an endogenous gene and thereby suppress the expression of the mutant allele. The kit may also contain one or more replacement nucleic acids.

Alternatively, a kit is provided for the detection of a polymorphism in a DNA, RNA, or body fluid sample by selective cleavage by a ribozyme that binds only to DNA or RNA comprising certain alleles of a polymorphism, wherein only those alleles that bind to the ribozyme are cleaved. The invention provides a diagnostic kit for detecting whether a patient has a mutation on the same allele as a polymorphism, e.g., by using long-range RT PCR.

In a particularly preferred embodiment, the genetic disease associated with the polymorphism is dominant negative or polygenic in nature, such as for example, osteogenesis imperfecta and retinitis pigmentosa.

### BRIEF DESCRIPTION OF THE DRAWINGS

The foregoing and other objects, features and advantages of the present invention, as well as the invention itself, will be more fully understood from the following description of preferred embodiments when read together with the accompanying drawings, in which:

FIG. 1 provides a schematic of two scenarios of the invention in which the allele suppression and replacement strategy may be utilized. A. Schematic of two alleles of a patient with a dominant negative mutation (indicated with an X) who is homozygous for a polymorphism (indicated with an o). Both alleles have a polymorphic variant which can be suppressed, or partially suppressed with a suppressor effector. A replacement construct comprising a replacement nucleic acid may need to be administered. The replacement construct contains a different polymorphic variant (indicated with an ▪) which can not be suppressed or is suppressed less efficiently by the suppressor effector and encodes wild-type protein or non-disease causing protein. B. Schematic of two alleles of a patient with a dominant negative mutation (indicated with an X) in one allele and is heterozygous for a polymorphism (indicated with an o or ▪). Both alleles have different polymorphic variants of a gene. The suppressor effector may selectively suppress the mutant allele. The allele with the mutation has a suppressible or partially suppressible polymorphism (indicated with an o) and the wild-type allele has a polymorphic variant (indicated with a ▪) which can not be suppressed or is suppressed less efficiently by the suppressor effector. To help overcome possible ill effects due to haploinsufficiency of a target gene, a replacement construct which includes a replacement nucleic acid may need to be administered. The replacement construct which encodes wild-type protein or non-disease causing or non-disease predisposing protein will have the polymorphic variant which can not be suppressed or is suppressed less efficiently.

FIG. 2A illustrates the retroviral plasmid pLRNL. RzPolCol1A1 was cloned into the SalI and BamHI sites of pLRNL. In addition, a CMV promoter driving a second RzPolCol1A1 was cloned into the ClaI site of pLRNL.

FIG. 2B illustrates that the RNA population generated by pLRNL would be a mixed population of three different RNAs due to the presence of three different promoters.

FIG. 3 illustrates graphs of percentage T-allele RNA collagen 1A1 cleaved by RzPolCol1A1 versus time. (A.) Curves a-g show percentage T-allele RNA cleavage by RzPolCol1A1 at different time intervals of timepoint cleavage reaction of T-allele RNA:RzPolCol1A1 of 1:0.1, 1:0.3, 1:0.5, 1:1, 1:2, 1:10 and 1:100, respectively. (B). Curves a-e show the steady-state interval for ratios of T-allele RNA:RzPolCol1A1 of 1:0.1, 1:0.3, 1:0.5, 1:1 and 1:2, respectively. The steady-state interval is at least 10 minutes. Vₘₐₓ and Kₘ were determined using the slopes of the graphs of cleavage of the T-allele RNA by RzPolCol1A1 under multiple-turnover conditions, where the ratio of T-allele RNA:RzPolCol1A1 is 1:0.1, 1:0.3 and 1:0.5. t_{1/2}, at which half the substrate COL1A1 transcript has been cleaved under saturating conditions, was calculated from the slope of curves g and h in FIG. 3A.

FIG. 4 illustrates how RzPolCol1A1 in pLRNL (FIG. 2) suppresses the RNA from the T-allele of collagen 1A1 in mesenchymal progenitor stem cells (MPCs), which are homozygous for the T-allele. FIGS. 4A and 4B show that the ribozyme suppresses the collagen 1A1 transcript by about 28%. The machine used in this experiment was the light cycler (Roche) and the kit used for the one step RT-PCR reaction was the QuantiTect SYBR Green RT-PCR kit (Qiagen, catalogue number 204243). FIG. 4A illustrates the amount of fluorescence (y-axis), representing the amount of initial gene transcript (in this case either collagen 1A1, collagen 1A2 or GAPDH), in an untransduced MPC line (mpc) and an MPC line stably transduced with pLRNL and RzPolCol1A1 (pol) (FIG. 2). All reactions were carried out in triplicate. Lanes 1-4 represent a standard curve of an RT-PCR reaction on RNA from mpc using collagen 1A1 primers. Lanes 4-7 represent RT-PCR reactions on RNA from mpc using collagen 1A1 primers. Lanes 8-10 represent RT-PCR reactions on RNA from pol using collagen 1A1 primers. Lanes 11-13 represent RT-PCR reactions on RNA from mpc using collagen 1A2 primers. Lanes 14-16 represent RT-PCR reactions on RNA from mpc using collagen 1A2 primers. Lanes 23-25 represent RT-PCR reactions on RNA from mpc using GAPDH primers. Lanes 26-28 represent RT-PCR reactions on RNA from pol using GAPDH primers. Lanes 29-32 are water blank reactions. It is clear that while the amount of collagen 1A1 is lower in pol than in mpc, the level of the two control genes, collagen 1A2 and GAPDH are similar in both pol and mpc. FIG. 4B illustrates the quantified amount of fluorescence measured by the light cycler (Roche). The lane numbers are as in FIG. 4A. From these numbers the suppression level of collagen 1A1 by RzPolCol1A1 is calculated to be 28.8%.

FIG. 5 shows GIF images of MPC cells after four days of growth. Cells were then treated with a primary anti-human type 1 collagen antibody raised in mouse and a secondary anti-mouse antibody with a green fluorescent lable. Cells were analysed and photographed with a fluorescent microscope. Green patches represent type 1 collagen protein (i.e. COL1A1 and COL1A2 fibers).

FIG. 6 shows GIF images of Pol 1,2 cells after four days of growth. Cells were then treated with a primary anti-human type 1 collagen antibody raised in mouse and a secondary anti-mouse antibody with a green fluorescent lable. Cells were analysed and photographed with a fluorescent microscope. Green patches represent type 1 collagen protein (i.e. COL1A1 and COL1A2 fibers).

FIG. 7 shows GIF images of Pol 3 cells after four days of growth. Cells were then treated with a primary anti-human type 1 collagen antibody raised in mouse and a secondary anti-mouse antibody with a green fluorescent lable. Cells were analysed and photographed with a fluorescent microscope. Green patches represent type 1 collagen protein (i.e. COL1A1 and COL1A2 fibers).

FIG. 8 shows GIF images of MPC cells after seven days of growth. Cells were then treated with a primary anti-human type 1 collagen antibody raised in mouse and a secondary anti-mouse antibody with a green fluorescent table. Cells were analysed and photographed with a fluorescent microscope. Green patches represent type 1 collagen protein (i.e. COL1A1 and COL1A2 fibers).

FIG. 9 shows GIF images of Pol 1,2 cells after seven days of growth. Cells were then treated with a primary anti-human type 1 collagen antibody raised in mouse and a secondary anti-mouse antibody with a green fluorescent table. Cells were analysed and photographed with a fluorescent microscope. Green patches represent type 1 collagen protein (i.e. COL1A1 and COL1A2 fibers).

FIG. 10 shows GIF images of Pol 3 cells after seven days of growth. Cells were then treated with a primary anti-human type 1 collagen antibody raised in mouse and a secondary anti-mouse antibody with a green fluorescent table. Cells were analysed and photographed with a fluorescent microscope. Green patches represent type 1 collagen protein (i.e. COL1A1 and COL1A2 fibers).

### DETAILED DESCRIPTION OF THE INVENTION

Reagents and methods are provided that circumvent the problem of genetic heterogeneity present in many genetic disorders by suppressing a polymorphic allele of a gene using one or more suppression effectors. Suppression effectors (or “RNA inactivating agents”) include nucleic acids such as ribozymes, DNA and RNA, PNAs, peptides, antibodies or modified forms of these used to silence or reduce gene expression in a sequence specific manner. The invention is of particular use in the suppression of gene alleles that contain mutations that are independent of the polymorphism and which cause abnormal or deleterious cell functioning, deleterious effect(s) or cell death. Suppression effectors can be directed to coding sequence, 5′ or 3′ untranslated regions (UTRs), introns, control regions, or to sequences adjacent to a gene or to any combination of such regions of a gene or RNA product. Binding of the suppression effector(s) prevents or lowers functional expression of a target allele of the endogenous gene carrying a deleterious mutation by targeting polymorphism(s) within or adjacent to the gene. In a preferred embodiment, the invention provides ribozymes and uses thereof which are designed to elicit cleavage of RNAs encoded by a target allele of a polymorphic gene. The invention further provides nucleotides and uses thereof which form triple helix DNA. Nucleic acids for antisense, ribozymes and triple helix DNA may be modified to increase their stability, binding efficiencies and/or uptake.

The term “target allele” “suppression effector target” or “target sequence” or the like are used interchangeably herein and refer to the allele of a gene which is targeted by a suppressor effector and which is thereby suppressed in the practice of the methods of the invention. In a preferred embodiment, the gene is an endogenous or naturally occurring gene. To “target” a gene or allele of a gene also refers to the binding of the suppression effectors of the invention to their targets, such as, for example, a ribozyme to an RNA encoded by a particular polymorphic allele of a gene.

Generally, suppression effectors such as nucleic acids—antisense or sense, ribozymes, PNAs, triple helix forming oligonucleotides, peptides and/or antibodies directed to polymorphisms in a gene, in transcripts or in protein, can be employed in the invention to achieve gene suppression. The invention is of particular use in the suppression of gene alleles that contain mutations that are independent of the polymorphism (e.g., a polymorphism is present on an allele and, in addition, a disease-causing mutation is present at a different position on that allele). Alleles of a specific gene can differ from each other in a single nucleotide, or more than one nucleotide, and can include substitutions, deletions, insertions and inversions of nucleotides.

In addition, a replacement nucleic acid may be provided which contains the sequence of a different polymorphic allele to that of the target allele to be suppressed. Preferred replacement nucleic acids include DNA or RNA. A replacement nucleic acid may be a partial sequence (i.e., encoding only part of an endogenous gene sequence) or a complete sequence. The replacement nucleic acid is therefore not recognized (i.e., is not bound by) or is recognized less effectively by the suppression effector. Replacement nucleic acids not only escape suppression, at least in part, but are likely to be translated into wild-type (e.g., non-disease causing or normal) gene product. Preferred replacement nucleic acids are operatively linked to appropriate promoter and/or enhancer sequences that direct efficient gene transcription, and/or sequences that ensure the stability, translation, and transport of the replacement nucleic acid RNA and its translation into protein. Additionally, inducible promoters could drive expression of replacement nucleic acids. Replacement genes may contain both naturally occurring and/or artificially introduces polymorphism(s).

In a preferred embodiment of the invention, methods are provided for the targeting (i.e., hybridize, detect, recognize) of a mutant allele at a polymorphic site within an endogenous gene that gives rise to a dominant or deleterious effect or phenotype. The deleterious effect may be either monogenic or polygenic. In another embodiment, a replacement nucleic acid is provided in the method of the invention to substitute the mutant gene product with a wild-type gene product.

In accordance with the invention a reagent is prepared, broadly referred to herein as a suppression effector, which has two functions. First, it comprises a nucleic acid having a nucleotide sequence complementary to a region of the sequence of the target allele that distinguishes that allele from its counterpart or from a properly functioning allele. Typically, the region is an allele-specific polymorphic site. Second, the reagent has a portion that functions to suppress or inhibit transcription or translation of the target allele or its mRNA. Both functions may be embodied in a single molecular structure, but the suppression effector may have distinct portions which provide a targeting function and a suppressor function. The targeting function is a nucleic acid that hybridizes under physiological conditions to the distinctive portion of the target allele or its RNA. The suppressing function preferably is achieved by a ribozyme which restricts or cuts the target allele or more preferably its RNA. In the case where the reagent is a site-specific ribozyme, it preferably is provided to a cell by transfection of a DNA which upon transcription generates the RNA structure of the ribozyme, complete with its targeting nucleotide sequence. Details of how to make and how to use such suppression effectors are disclosed herein. However, it should be noted that the means and methods of transfecting such DNAs into cells, using appropriate vectors and otherwise, and the methods for targeting the transfection to appropriate cells in a multicellular organism, are known to those skilled in the art and are not considered an aspect of the invention.

As a result of this transfection, expression of the target allele is inhibited, suppressed, or preferably eliminated, and its normally consequent phenotypic effects are blocked or at least diminished. In the case where the target allele's counterpart is wild-type or otherwise is expressed so as to maintain in the organism a non-diseased phenotype, nothing further may be needed to complete the therapeutic regime. In other instances, a replacement gene may be necessary. The replacement gene may be supplied to cells via coadministration on the same vector as, or at the same time as the DNA encoding the suppression effector. This is otherwise generic gene therapy of the type described, for example, in 5,399,346, 5,087,617, 5,246,921, 5,834,440, the disclosures of which are incorporated herein by reference.

The invention provides methods for suppressing specifically, selectively, or preferentially one allele of an endogenous gene with a deleterious mutation(s) and, if required, introducing a replacement nucleic acid, the method having the steps of: (1) providing a suppressor effector that binds to at least one allele of a gene to be suppressed and (2) providing a replacement nucleic acid which is a different allele (either a naturally occurring or artificially derived allelic variant) than the allele targeted for suppression, wherein the suppressor effectors are unable to bind or bind less efficiently to equivalent or homologous regions in the genomic DNA or cDNA to prevent expression of the replacement gene. A suppressor effector which partially recognizes its target DNA or target allele may not completely suppress its target allele. In a preferred embodiment a suppression effector achieves between 5% and 10%, 10% and 30%, 30% and 60% suppression of its target gene, more preferably between 60% and 80% suppression, more preferably 80%-90% suppression and still more preferably 90% to 100% suppression. The replacement nucleic acids will not be recognised by suppression nucleic acids, or will be recognised less effectively, than the allele targeted by suppression nucleic acids.

Replacement nucleic acids are provided such that replacement nucleic acids will not be recognised by naturally occurring suppressors found to inhibit or reduce gene expression in one or more individuals, animals or plants. The invention provides for use of replacement nucleic acids which have altered sequences around polymorphic site(s) targeted by suppressors of the gene such that suppression by naturally occurring suppressors is completely or partially prevented. This may be due to partial or less efficient recognition, or selective or preferential bind of supressor to the mutant allele vs. the replacement allele, and may refer to binding which is not stable, due to, for example, sequence dissimilarity or lack of complementarity of the sequences. Replacement genes may have naturally occurring or artificially introduced polymorphisms to minimize suppression.

In an additional embodiment of the invention, a method is provided for suppressing one allele of a gene using polymorphism where that allele or the product of that allele interferes with the action of an administered compound, e.g., by increasing the effectiveness or action of a compound with which it is co-administered. It is clear that variations in a range of genes and therefore gene products can significantly alter drug response. There are, for example, instances where one allelic variant of a drug metabolising enzyme (DME) either metabolises an administered drug too rapidly thereby limiting the bioavailability of the drug and therefore its efficacy or alternatively some allelic variants of DMEs metabolise the administered drug too slowly therefore leading to potential toxicity—i.e., too high levels of drug. Hence, it may be that the co-administration of the drug together with suppressing one variant of the gene encoding the DME and replacing it with an alternative allelic variant of the DME would aid in optimizing the effectiveness of the co-administered drug and limiting associated toxicity. The same principle applies to genes and gene products involved in either the absorption and transport of the drug and/or the receptor target for the drug itself. Some key categories of genes include those encoding drug metabolising enzymes (for example, the cytochrome P450 genes, thiopurine methyl transferase amongst others), genes encoding receptor(s) for drugs (for example, dopamine receptors, β2-adrenergic receptor amongst others) and genes encoding products which alter drug absorption and transport (for example, P-glycoprotein amongst others). For example, the multi-drug resistance gene (MDR-1) encoding P glycoprotein, a member of the ABC transporter family, can significantly influence the bioavailability of chemotherapeutic drugs and over-expression of this gene is responsible for tumour resistance to chemotherapeutics in some cases (Refs: Gottesman M M, Pastan I. Annu Rev Biochem 62: 385-427 1993). Similarly, well over 100 drugs are substrates for one of the cytochrome P450 genes (CYP2D6); various allelic variants have been defined in this gene that can result in significantly altered activity of the encoded protein, for example, allele CYP2D6*5 carries a deletion and hence encodes no enzyme (Skoda R C et al. 1988 PNAS USA 85: 5240-5243; Daly A K et al. 1996 Pharmacogenetics 6: 193-201). Furthermore, studies with the β2-adrenergic receptor gene suggest that a single polymorphic variant at codon 16 (Gly/Arg) of the receptor gene significantly alters response (approximately a 5-fold difference) to bronchodilators such as albuterol (Martinez F D et al. 1997 J Clin Invest 1140: 3184-3188). Hence, the suppression and replacement of genes involved in altering drug response may aid in optimizing the utility of a broad range of drugs. In another embodiment, functional gene expression of the target allele of a gene interferes with the action of an administered compound.

In another embodiment, the suppression effector and replacement technology can be used to render a cell or individual which is genetically predisposed to infection by an infectious agent resistant to infection. It is clear that infectious agents use defined molecular mechanisms to enter and infect cells. These mechanisms are depending on/specific to a particular infectious agent and indeed in some cases can vary between different serotypes of the same infectious agent (Davidson et al. 2000 PNAS 97: 3428-3432; Yotnda P et al. 2001 Gene Therapy 8: 930-937). There is also evidence that small variations in the genes encoding products involved in these mechanisms of infection can have a substantial effect(s) on the ability of the agent to be infectious. For example, there is evidence that HIV requires the CCR5 receptor for infection. The CCR5 gene encodes a cell surface receptor protein that binds HIV-suppressive β-chemokines. It has been observed that some individuals seem to be resistant to HIV (Samson et al. 1996 Nature 382: 722-725) and this resistance has been linked to one allelic variant of the CCR5 gene which has a 32 bp deletion in the gene—individuals homozygous for this allele seem to be highly resistant to HIV infection. In Caucasian population the frequency of this allele is believed to be about 0.1 suggesting that approximately 1 in 100 people may be homozygous for the allele. The other 99% of the population harbour one or two alleles of the CCR5 receptor gene that aids HIV infection. Similarly it has been established that the Haemaglobin C variant (β6Glu to Lys) can protect against malarial infection in individuals who are Haemoglobin C homozygous (HbCC). (Modlano D et al. 2001 Nature 414: 305-308/Commentary in Science magazine 2001 294: p1439). Given this scenario there has been and will continue to be a natural evolution towards increased frequencies of the Haemaglobin C variant in populations where malaria is prevalent. Given knowledge of the molecular mechanisms of infection and resistance to infectious agents the suppression and replacement technologies described herein can be used in the treatment of infectious agents/disorders such as those outlined above.

The invention further provides a tool for selectively suppressing a mutant endogenous gene or an endogenous gene which predisposes to disease by the introduction of nucleic acids targeting one allele of a gene to be suppressed, wherein suppression is targeted to polymorphism(s) in coding regions 5′ or 3′ untranslated regions, intronic regions, control regions of a gene to be suppressed or regions adjacent to a gene to be suppressed. The method may also include the sequential or concomitant introduction of replacement nucleic acids with sequences which allow its expression. In an embodiment, the suppressor may be administered with additional compounds.

In another aspect, the invention provides a kit containing suppression effectors and replacement nucleic acids for use in the treatment of a disease caused by a deleterious mutation in a gene. In one embodiment, suppression effectors able to bind one allelic variant of the gene to be suppressed are provided and, if required, a replacement nucleic acid which has control regions that allow its expression. The suppression effector completely or partially escapes suppression.

The invention further provides a vector or vectors (e.g., expression vectors) comprising suppression effectors in the form of nucleic acids directed towards a polymorphic site within or adjacent to a target gene and vector(s) containing genomic DNA or cDNA encoding a replacement gene sequence to which suppression nucleic acids are unable to bind (or bind less efficiently), in the preparation of a method of suppression and replacement in cells harboring dominant negative mutations. Exemplary viral vectors which may be used in the practice of the invention include those derived from adenovirus (Ad) (Macejak et al. 1999); adenoassociated virus (AAV) (Horster et al. 1999); retroviral-C type such as MLV (Wang et al. 1999); lentivirus such as HIV or SIV (Takahashi et al. 1999); herpes simplex (HSV) (Latchman et al. 2000); and SV40 (Strayer et al. 2000). Exemplary, non-viral vectors which may be useful in the practice of the invention include bacterial vectors from Shigella flexneri (Sizemore et al. 1995 and Courvalin et al. 1995), such as the S. flexneri which is deficient in cell-wall synthesis and requires diaminopimelicacid DAP) for growth. In the absence of DAP, recombinant bacteria lyse in the host cytosol and release the plasmid. Cationic lipid mediated delivery of suppression effectors (Tam et al. 2000), soluble biodegradable polymer-based delivery (Maheshwari et al. 2000), or electroporation/ionthophoresis (Muramatsu et al. 2001; Rossi et al. 1983) may also be used.

Nucleic acids for suppression or replacement gene nucleic acids may be provided in the same vector or in separate vectors. Naked nucleic acids or nucleic acids in vectors can be delivered with lipids or other derivatives which aid gene delivery. Nucleotides may be modified to render them more stable, for example, resistant to cellular nucleases while still supporting RNaseH mediated degradation of RNA or with increased binding efficiencies.

The invention also relates to a strategy for suppressing a gene or disease allele using methods which do not target the disease mutation specifically but instead target some characteristic associated with the allele in which the disease mutation resides. “Characteristic” refers to any nucleotide or sequence difference between two alleles of a gene or the gene product or the phenotype associated therewith. A particular embodiment of the invention is the use of polymorphism within a gene to direct suppression strategies to the disease allele while still allowing continued expression of the normal allele. The strategy circumvents the need for a specific therapy for every mutation within a given gene. In addition the invention allows greater flexibility of choice of target sequence for suppression of a disease allele.

Generally the present invention will be useful where the gene, which is naturally present in the genome of a subject, contributes to a disease state. Generally, one allele of the gene in question will be mutated, that is, will possess alterations in its nucleotide sequence that affects the function or level of the gene product. For example, the alteration may result in an altered protein product from the wild-type gene or altered control of transcription and processing. Inheritance, or somatic acquisition, of such a mutation can give rise to a disease phenotype or can predispose an individual to a disease phenotype. Alternatively, the gene of interest could also be of wild-type or normal phenotype, but contribute to a disease state in another way such that the suppression of the gene would alleviate or improve the disease state or improve the effectiveness of an administered therapeutic compound.

Notably, the invention has the advantage that the same suppression strategy when directed to polymorphisms could be used to suppress, in principle, many mutations in a gene. This is particularly relevant when large numbers of mutations within a single gene cause disease pathology. The proportion of disease mutations which can be suppressed using a polymorphism will depend in part on the frequency of the polymorphism chosen for suppression in the population. For example, approximately 40% of individuals are heterozygous for the collagen 1A1 3210 polymorphism. To increase the number of individuals that could be treated using suppression effectors directed to polymorphisms and, in addition, to increase the efficiency of suppression, multiple polymorphisms within a gene could be used when necessary.

Suppression using one allele of a polymorphism enables, when necessary, the introduction of a replacement gene with a different allele of the polymorphism such that the replacement gene escapes suppression completely or partially as does the normal endogenous allele. The replacement gene provides (when necessary) additional expression of the normal protein product when required to ameliorate pathology associated with reduced levels of wild-type protein. The same replacement gene could in principle be used in conjunction with the suppression of many different disease mutations within a given gene. Target polymorphisms may be found either in coding or non-coding sequence or in regions 5′ or 3′ of the gene. For example, intronic polymorphisms could be used for suppression. The use of polymorphic targets for suppression in 5′ and 3′ non-coding sequence holds the advantage that such sequences are present in both precursor and mature RNAs, thereby enabling suppressors to target all forms of RNA. In contrast, intronic sequences are spliced out of mature transcripts. Similarly polymorphism found in coding sequence would be present in precursor and mature transcripts, again enabling suppressors to target all forms of RNA. Polymorphisms in coding sequence may be silent and have no effect on subsequent protein amino acid content or may result in an amino acid substitution but not lead to a disease pathology. In the latter case, such polymorphisms may enable targeting of one allele specifically at the protein level by directing, for example, antibodies, uniquely to one form of the protein. In addition, polymorphisms may cause disease or predispose to disease.

The instant invention is particularly useful for diagnosing an autosomal dominant or polygenic genetic disorder by detecting the presence of a polymorphism that is independent of a disease mutation on an allele. An individual may be homozygous or heterozygous for a particular polymorphism. The proportion of individuals who will be heterozygous for a particular polymorphism will depend on the allele frequencies of the polymorphism in the population being assessed.

In some cases it is possible that lowering RNA levels may lead to a parallel lowering of protein levels, however this is not always the case. In some situations mechanisms may prevent a significant decrease in protein levels despite a substantial decrease in levels of RNA. However, in many instances suppression at the RNA level has been shown to lower protein levels. In some cases it is thought that ribozymes elicit suppression not only by cleavage of RNA but also by an antisense effect due to the antisense arms in the ribozyme surrounding the catalytic core. Ribozyme activity may be augmented by the use of, for example, non-specific nucleic acid binding proteins or facilitator oligonucleotides (Herschlag et al., 1994; Jankowsky and Schwenzer, 1996). Multitarget ribozymes such as, for example, connected or shotgun ribozymes have been suggested as a means of improving the efficiency of ribozymes for gene suppression (Ohkawa et al., 1993). In addition, maxizymes which do not require NUX sites offer more flexibility in terms of selecting suitable target sites.

In an embodiment, the invention provides transgenic animals, e.g., non-human animals, birds, reptiles, marsupials or amphibians, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. This molecule may be integrated within a chromosome, or it may be extrachromosomally replicating DNA. In the typical transgenic animals described herein, the transgene causes cells to express a recombinant form of a polypeptide, e.g. either agonistic or antagonistic forms. Moreover, transgenic animals may be animals in which gene disruption of one or more genes is caused by human intervention, including both recombination and antisense techniques. The technologies could be used, as in humans, to develop therapies for animals. Alternatively, the patented technologies could be used as research tools in the development of animal models mostly via transgenic techniques. In addition, they could be utilized in such animals to investigate the role/functions of various genes and gene products. Alternatively, the methods of the invention herein can be used in the field of veterinary medicine as therapeutics.

### Synthesis of an Expression Effector

A ribozyme can be designed to cleave most RNA molecules by designing specific ribozyme arms which bind to a particular RNA on either side of a consensus NUX site, where N is selected from the group consisting of C, U, G, A and X is selected from the group consisting of C, U or A. Thus any RNA possessing an NUX site is a potential target. However, other variables require consideration in designing a ribozyme, such as the two dimensional conformation of the RNA (e.g., loops) and the accessibility of a ribozyme for its target. The utility of an individual ribozyme designed to target an NUX site in an open loop structure of transcripts from one allele of a gene will depend in part on the robustness of the RNA open loop structure when various deleterious mutations are also present in the transcript. Robustness may be evaluated using an RNA-folding computer program such as RNAPlotFold. A robust loop refers to the occurrence of the loop for most or all of the plotfolds with different energy levels. For example, in Example 1, data for six different adRP causing mutations in the rhodopsin gene were evaluated. For each of these mutations the large RNA open loop structure which is targeted by Rz10 was maintained in the mutant transcripts (Table 1).

Robustness of loop structures was evaluated over a broad energy profile, for example, a 140 bp RNA was assessed in the range between about −280 to −380 kcal/mol. For example, a 1347 nucleotide human COL1A1 mRNA was assessed for internal molecular energy levels of: −311.8, −312.9, −313.0, −314.1 and −314.4 kcal/mole. A 370 nucleotide human COL1A1 mRNA was assessed in a second plotfold for levels of: −52.5, −51.7, −51.1, −50.7 and −50.2 kcal/mole. A 1399 nucleotide human COL1A2 mRNA was assessed for: −455.4, −455.2, −454.1, −453.9 and −453.7 kcal/mole. A 1300 nucleotide human rhodopsin mRNA was assessed for: −342.3, −343.0, 0342.7, −342.5, −341.9, −341.1, −340.4, −340.3, −340.1, −339.3, −338.3 and −338.1 kcal/mole.

As exemplified herein, RNA was expressed from cDNAs coding for alleles of three different proteins: human rhodopsin, human type I collagen 1A1 and human type I collagen 1A2. The cDNA templates utilized in the invention coded for specific allelic variants of each of these three genes. In the case of rhodopsin, polymorphisms were artificially derived to exemplify the invention and the use of the invention for retinopathies such as adRP. In the case of the collagen cDNAs, three separate naturally occurring polymorphisms of the human collagen 1A1 or 1A2 genes were targeted to demonstrate the invention and the potential use of the invention for disorders such as OI. Transcripts expressed from individual allelic variants of all three genes were used as in vitro targets of hammerhead ribozymes directed towards one single allelic form of the gene. In all three examples, the ribozymes directed to polymorphic sites were successful in cleaving target RNAs from one allele in the predicted manner. Additionally, replacement transcripts from an alternative polymorphic variant of each of the genes tested were not cleaved by the ribozymes, thus demonstrating the polymorphism-specific nature of the reagents and methods of the invention.

In one embodiment, the invention is exemplified herein using polymorphic regions of the rhodopsin gene (see Example 1). The unmutated human rhodopsin cDNA (SEQ ID NO: 1) and the human rhodopsin cDNA with a single nucleotide substitution in the coding sequence were cut with BstEII and expressed in vitro. A single base change occurs at the third base position of the codon at position 477 (position 209 of SEQ ID NO: 12) and therefore does not alter the amino acid coded by this triplet. While the polymorphism is artificially derived, it mirrors naturally occurring polymorphisms in many genes which contain single nucleotide alterations that are silent. The hammerhead ribozyme Rz10 (SEQ ID NO:14) cloned into pcDNA3 was cut with XbaI and expressed in vitro. Resulting ribozyme and human rhodopsin RNAs were mixed with varying concentrations of MgCl₂ to optimize cleavage of template RNA by Rz10. In addition, a profile of human rhodopsin RNA cleavage by Rz10 over time was generated by polyacrylamide gel electrophoresis by standard methods. Unmutated and mutated human rhodopsin cDNAs were cut with FspI and BstEII respectively, expressed in vitro and the resulting transcripts mixed together with Rz10 RNA to test for cleavage over time. Likewise, unmutated and mutated human rhodopsin cDNAs were cut with Acyl and BstEII respectively, both were expressed in vitro and resulting transcripts mixed with Rz10 RNA at varying MgCl₂ concentrations to test for cleavage. It is worth noting that the Acyl enzyme cuts after the stop codon and therefore the resulting RNA included the complete coding sequence of the gene. In all cases expressed RNAs were the predicted size. In addition, in all cases unmutated transcripts were cleaved into products of the predicted size. Cleavage of unmutated human rhodopsin RNA was almost complete—little residual uncleaved RNA remained (e.g., less than 5%). In all cases mutated human rhodopsin RNAs with a single base change at a silent site remained intact, that is, it was not cleaved by Rz10 and therefore “protected.” Thus, transcripts from one allele of this artificial polymorphism were cleaved by Rz10 while transcripts from the other allele were not cleaved.

The ribozyme Rz20 (SEQ ID NO: 15) was designed to elicit mutation specific cleavage of transcripts containing a Pro23Leu rhodopsin mutation. Rz20 cloned into pcDNA3 was cut with XbaI and expressed in vitro. Similarly, the rhodopsin cDNA containing a Pro23Leu mutation was cut with BstEII and expressed in vitro. A profile of human rhodopsin RNA cleavage by Rz20 over time was generated by polyacrylamide gel electrophoresis by standard methods. All expressed products, and cleavage products were the predicted size

Cleavage of mutant rhodopsin transcripts by Rz10 which targets a ribozyme cleavage site 3′ of the site of the Pro23Leu mutation in one allele of an artificially derived polymorphism in rhodopsin coding sequence was also explored. The mutant rhodopsin cDNA and Rz10 clones were cut with BstEII and XbaI respectively and expressed in vitro. Resulting RNAs were mixed and incubated with 10 mM MgCl₂ for varying times. All expressed products and cleavage products were the predicted size. Rz10 cleaved mutant rhodopsin transcripts when the mutation was on the same allele of the polymorphism targeted by Rz10. Using an artificially derived allelic variant around the Rz10 cleavage site, transcripts from the artificial allele remained intact due to the absence of the Rz10 target site. Hence, Rz10 can be used to cleave mutant transcripts in a manner independent of the disease mutation itself, that is, by targeting a polymorphism, while wild-type transcripts from the alternative allele (in this case artificially derived to exemplify the process for rhodopsin) remain intact and therefore could supply the wild-type protein.

Table 1A provides a list of some polymorphisms in the rhodopsin, peripherin and collagen 1A1 and 1A2 genes. Table 1B shows that the Rz10 target site (475-477) open loop structure of rhodopsin RNA remains intact in various mutant transcripts

TABLE 1
A: Listing of some polymorphisms (silent/non-silent) in rhodopsin,
peripherin and collagen 1A1 and 1A2 genes.
The polymorphisms used in the invention are listed here - however
many other polymorphisms have been characterised in the collagen
1A1 and 1A2 genes.
A 38 base pair polymorphism in collagen1A2 is also listed.

| Rhodopsin | Peripherin | Collagen 1A1 | Collagen 1A2 |
|---|---|---|---|
| Gly 120 Gly | C558T | T(0.28)3210C | A902G |
| | | (0.72) | |
| Ala 173 Ala | Glu 304 Gln | | T907A |
| | Lys 310 Arg | | 38 bp insert. |
| | | | (Dalgleish, 1986) |
| | Gly 338 Asp | | |

B: Rhodopsin mutations tested to assess if the predicted open
loop RNA structure containing the Rz10 target site (475-477)
remains intact in mutant transcripts.

| Rhodopsin mutation | RNA open loop targeted by Rz10 |
|---|---|
| Pro 23 Leu | Intact |
| Gly 51 Val | Intact |
| Thr 94 IIe | Intact |
| Gly 188 Arg | Intact |
| Met 207 Arg | Intact |
| IIe del 255 | Intact |

In another embodiment, the invention is exemplified herein using polymorphic regions of the collagen 1A1 gene (see Example 1). The hammerhead ribozyme RzPolCol1A1 (SEQ ID NO: 19) targeting a polymorphic site in human collagen 1A1 sequence was cut with XbaI and expressed in vitro. The human collagen 1A1 cDNA clones (A and B) containing the two allelic forms of a naturally occurring polymorphism (T/C) in the 3′UTR of the gene at position 3210 of the sequence were cut with XbaI, expressed in vitro and both RNAs mixed separately with RzPolCol1A1 RNA to test for cleavage. RNAs were mixed with varying concentrations of MgCl₂ to optimize cleavage of RNAs by RzPolCol1A1. Notably, over 90% of the RNA transcripts from human collagen 1A1 (A) which has a T nucleotide at position 3210 and therefore contains a ribozyme cleavage site GTC (3209-3211) were cleaved, while transcripts from the other allele (collagen 1A1 (B)) which has a C nucleotide at this position, remained intact. Cleavage of collagen 1A1 transcripts over time in 10 mM MgCl₂ was assessed for the T allele of the polymorphism and the C allele of the polymorphism at position 3210, as well.

In another embodiment, the invention is exemplified herein using polymorphic regions of the collagen 1A2 gene (see Example 1). The hammerhead ribozymes Rz902 (SEQ ID NO:23) and Rz907 (SEQ ID NO:24), cloned into pcDNA3, which target two different polymorphic sites located 6 bases apart in human collagen 1A2 sequence. The two polymorphic sites are in the same open loop structure in the predicted 2-D conformations of the collagen 1A2 transcript. One ribozyme targets one allele of the first polymorphism in collagen 1A2 while the second ribozyme targets one allele of the second polymorphism in collagen 1A2. This experiment demonstrates that multiple polymorphisms within or close to a gene in the same allele could be targeted to achieve efficient and specific suppression of an individual allele. Significantly, naturally occurring polymorphic variants have been observed in the retinal specific genes encoding the photoreceptor proteins rhodopsin and peripherin (Table 1). Although these do not occur at optimal ribozyme cleavage sites (i.e., NUX sites in RNA open loop structures) approaches such as, for example, antisense, triplex helix, maxizymes or antibodies may be utilized to achieve suppression of single alleles carrying disease mutations while enabling continued expression from alternative allelic forms of the gene with wild-type sequence using these or other polymorphisms. Further sequencing of the introns of these retinal genes may reveal other appropriate polymorphic target sites for hammerhead ribozymes. The human collagen 1A2 cDNA clones (A and B) containing two allelic forms of two polymorphisms in the coding sequence of the gene, at positions 902 and 907 of the sequence, were both cut with either XbaI and MvnI, expressed in vitro and RNA, mixed together with Rz902 or Rz907 RNA to test for cleavage of transcripts by these ribozymes. All expressed transcripts were the predicted sizes. RNAs were mixed with varying concentrations of MgCl₂ to optimize cleavage of RNAs by Rz902 and Rz907. Notably the majority of the RNA transcripts from human collagen 1A2 (A) which has a G nucleotide at position 902 and a T nucleotide at position 907 was cleaved by Rz907. Cleavage products were the predicted size. In contrast human collagen 1A2 (A) transcripts were not cleaved by Rz902. This allelic form of the gene has a ribozyme cleavage site at 907 but does not have a cleavage site at position 902. Notably the situation is reversed with transcripts from human collagen 1A2 (B) where in this allelic form of the gene due the nature of the polymorphisms present there is a ribozyme cleavage site at position 902 but the site which in the other allelic form of the gene was at position 907 has been lost. Transcripts from human collagen 1A2 (B) were cleaved specifically by Rz902 and cleavage products were the predicted size. In contrast, transcripts from this allelic form of the gene were not cleaved by Rz907 due to the alteration in the sequence around the ribozyme cleavage site. Cleavage of collagen 1A2 (B) by Rz902 was less efficient than cleavage of collagen 1A2 (A) by Rz907. This is consistent with 2-D predictions of RNA open loop structures for the two polymorphisms—in the allele containing the Rz907 ribozyme cleavage site, the target site is found more consistently in an open loop structure when compared to the Rz902 cleavage site. However, these two polymorphisms which are in strong linkage disequilibrium with each other (separated by only 6 bases) and which are often found in the same open loop structure of the transcript clearly demonstrate the feasibility and utility of polymorphisms in directing suppression effectors to different alleles of genes, in this case the human collagen 1A2 gene.

### Ribozyme Suppressor

Preferred antisense molecules are ribozymes designed to catalytically cleave target allele mRNA transcripts to prevent translation of mRNA and expression of a target allele (See, e.g., PCT International Publication WO 94/11364, published Oct. 4, 1990; Sarver et al., 1990). Ribozymes are enzymatic RNA molecules capable of catalyzing the specific cleavage of RNA. A ribozyme may be, for example, a hammerhead ribozyme (Haseloff et al. 1989); a hairpin ribozyme (Feldstein et al. 1989); a hepatitis delta virus RNA subfragment (Wu et al. 1989); a neurospora mitochondrial VA RNA (Saville et al. 1990); a connected or shotgun ribozyme (Chen et al. 1992); or a minizyme (or a transplicing ribozyme (Ayre et al. 1999) or a maxizyme (Kuwabara et al. 1998) (Kuwabara et al. 1996). In addition, the inhibitory effect of some ribozymes may be due in part to an antisense effect of the antisense sequences flanking the catalytic core which specify the target site. A hammerhead ribozyme may cleave an RNA at an NUX site in any RNA molecule, wherein N is selected from the group consisting of C, U, G, A and X is selected from the group consisting of C, U or A. Alternatively, other recognition sites may be used as appropriate for the ribozyme. The mechanism of ribozyme action involves sequence specific hybridization of the ribozyme molecule to complementary target RNA, followed by an endonucleolytic cleavage. The composition of ribozyme molecules must include one or more sequences complementary to the target gene mRNA, and must include a catalytic sequence responsible for mRNA cleavage. For example, see U.S. Pat. No. 5,093,246, which is incorporated by reference herein in its entirety. As such within the scope of the invention are engineered hammerhead motif ribozyme molecules that specifically and efficiently catalyze endonucleolytic cleavage of RNA sequences encoding a target allele protein.

While various ribozymes that cleave mRNA at site specific recognition sequences can be used to destroy target allele mRNAs, the use of hammerhead ribozymes is preferred. Hammerhead ribozymes are small catalytic RNA enzymes that can elicit sequence specific cleavage of a target RNA transcript. Hammerhead ribozymes cleave mRNAs at locations dictated by flanking regions that form complementary base pairs with the target mRNA. The sole requirement is that the target mRNA have the following sequence of two bases: 5′-UX-3′ where X=A, C or U. The construction and production of hammerhead ribozymes is well known in the art and is described more fully in Haseloff and Gerlach, 1988.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984; Zaug and Cech, 1986; Zaug, et al., 1986; published International patent application No. WO88/04300 by University Patents Inc.; Been and Cech, 1986). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target allele. Hairpin, hammerhead, trans-splicing ribozymes and indeed any ribozyme could be used in the practice of the invention (Haseloff et al. 1989; Feldstein et al. 1989; Wu et al. 1989; Saville 1990; Chen et al. 1992; and Kuwabara et al 1996). In addition, any RNA inactivating or RNA cleaving agent which is capable of recognition of and/or binding to specific nucleotide sequences in an RNA is contemplated. For example, splicesome-mediated RNA trans-splicing (Puttaraju et al. 1999); double strand RNA (Fire et al. 1998; Bahramian et al. 1999); PNAs (Chinnery et al. 1999; Nielsen et al. 2000); antisense DNA (Reaves et al. 2000); antisense RNA (Chadwick et al. 2000); or triple helix forming oligonucleotides (Chan et al. 1997) have been used as gene therapies. All types of RNA may be cleaved in the practice of the invention, including, for example, mRNA, tRNA, rRNA and snRNPs.

As in the antisense approach, the ribozymes can be composed of modified oligonucleotides (e.g., for improved stability, targeting, etc.) and should be delivered to cells which express the target allele. A preferred method of delivery involves using a DNA construct “encoding” the ribozyme under the control of a strong constitutive pol III or pol II promoter, so that transfected cells will produce sufficient quantities of the ribozyme to destroy endogenous target allele messages and inhibit translation. Because ribozymes unlike antisense molecules, are catalytic, a lower intracellular concentration of ribozymes may be required for efficient suppression.

### Antisense Suppressor

One aspect of the invention relates to the use of the suppressor effectors in “antisense” suppression. As used herein, antisense suppression refers to administration or in situ generation of nucleic acid sequences or their derivatives which specifically hybridize or bind under cellular conditions, with the cellular mRNA and/or genomic DNA encoding one or more of the subject target alleles so as to inhibit expression of that target allele, e.g. by inhibiting transcription and/or translation. The binding may be by conventional base pair complementarity, or, for example, in the case of binding to DNA duplexes, through specific interactions in the major groove of the double helix. In general, antisense suppression refers to the range of techniques generally employed in the art, and includes any suppression which relies on specific binding to nucleic acid sequences. An antisense construct of the present invention can be delivered, for example, as an expression plasmid which, when transcribed in the cell, produces RNA which is complementary to at least a unique portion of the cellular mRNA which encodes a target sequence or target allele of an endogenous gene. Alternatively, the antisense construct is a nucleic acid which is generated ex vivo and which, when introduced into the cell causes inhibition of expression by hybridizing with the mRNA and/or genomic sequences of a target allele of an endogenous gene. Such nucleic acids are preferably modified oligonucleotides which are resistant to endogenous nucleases, e.g. exonucleases and/or endonucleases, and are therefore stable in vivo. Modifications, such as phosphorothioates, have been made to nucleic acids to increase their resistance to nuclease degradation, binding affinity and uptake (Cazenave et al., 1989; Sun et al., 1989; McKay et al., 1996; Wei et al., 1996). Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al., 1988 and Stein et al., 1988.

Antisense approaches involve the design of oligonucleotides (either DNA or RNA) that are complementary to a target allele of a gene or its gene product. The antisense oligonucleotides may bind to the target allele mRNA transcripts and prevent translation. Absolute complementarity, although preferred, is not required. Antisense nucleic acids that are complementary to the 5′ end of the message, e.g., the 5′ untranslated sequence up to and including the AUG initiation codon, should work most efficiently at inhibiting translation. However, sequences complementary to the 3′ untranslated sequences of mRNAs are also effective at inhibiting translation of mRNAs. (Wagner, R. 1994. Therefore, nucleic acids complementary to either the 5′ or 3′ untranslated, non-coding regions of a target allele of an endogenous gene could be used in an antisense approach to inhibit translation of the product of the target allele. Nucleic acids complementary to the 5′ untranslated region of the mRNA should preferably include the complement of the AUG start codon. Antisense nucleic acids complementary to mRNA coding regions are less efficient inhibitors of translation but could be used in accordance with the invention. Whether designed to hybridize to the 5′, 3′ or coding region of the mRNA encoding a target allele, antisense nucleic acids should be about at least six nucleotides in length, and are preferably nucleic acids ranging from 6 to about 50 nucleotides in length. In certain embodiments, the nucleic acid is at least 10 nucleotides, at least 17 nucleotides, at least 25 nucleotides, or at least 50 nucleotides in length.

Suppression of RNAs that are not translated are also contemplated, such as, for example, snRNPs, tRNAs and rRNAs. For example, some genes are transcribed but not translated or the RNA transcript functions at the RNA level (i.e., the RNA of these genes may have a function that is separate from the function which its translated gene product (protein) may have). For example, in an Irish family suffering from retinitis pigmentosa in conjunction with sensorineural deafness, the mutation was identified to be a single base substitution in the second mitochondrial serine tRNA gene, a gene which is indeed transcribed but not translated (Mansergh et al. 1999). Other examples include Tsix and Xist (van Stijn et al. 1995; Ruport et al. 1995), H19 (Miyatake et al. 1996; Matsumoto et al. 1994; Redeker et al. 1993), IPW (imprinted gene in the Prader-Willi syndrome region) (Wevrick et al. 1994). The IPW RNA is spliced and polyadenylated, but its longest open reading frame is 45 amino acids. The RNA is widely expressed in adult and fetal tissues and is found in the cytoplasmic fraction of human cells, which is also the case for the H19 non-translated RNA, but differs from the Xist RNA which is found predominantly in the nucleus. Using a sequence polymorphism, exclusive expression from the paternal allele in lymphoblasts and fibroblasts has been demonstrated and monoallelic expression found in fetal tissues.

Regardless of the choice of target sequence, it is preferred that in vitro studies are first performed to quantitate the ability of the antisense nucleic acid to inhibit gene expression. It is preferred that these studies utilize controls that distinguish between antisense gene inhibition and nonspecific biological effects of the nucleic acids. It is also preferred that these studies compare levels of the target RNA or protein with that of an internal control RNA or protein.

The antisense nucleic acids can be DNA or RNA or chimeric mixtures or derivatives or “modified versions thereof”, single-stranded or double-stranded. As referred to herein, “modified versions thereof” refers to nucleic acids that are modified, e.g., at a base moiety, sugar moiety, or phosphate backbone, for example, to improve stability or halflife of the molecule, hybridization, etc. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone. The nucleic acid may include other appended groups such as peptides (e.g., for targeting host cell receptors in vivo), or agents facilitating transport across the cell membrane (see, e.g., Letsinger et al., 1989; Lemaitre et al., 1987; PCT Publication No. WO 88/09810, published Dec. 15, 1988) or the blood-brain barrier (see, e.g., PCT Publication No. WO 89/10134, published Apr. 25, 1988), hybridization-triggered cleavage agents, (See, e.g., Krol et al., 1988) or intercalating agents. (See, e.g., Zon, 1988). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including, but not limited to, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), -5-methyl-2-thiouracil, 3-(3-amino-3-N-2-carboxypropyl) uracil, (acp3) w, and 2,6-diaminopurine.

The antisense nucleic acids may also comprise at least one modified sugar moiety selected from the group including but not limited to arabinose, 2-fluoroarabinose, xylulose, and hexose.

In yet another embodiment, the antisense nucleic acid comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense nucleic acid is an .alpha.-anomeric oligonucleotide. An .alpha.-anomeric oligonucleotide forms specific double-stranded hybrids with complementary RNA in which, contrary to the usual .beta.-units, the strands run parallel to each other (Gautier et al., 1987). The oligonucleotide is a 2′-0-methylribonucleotide (Inoue et al., 1987), or a chimeric RNA-DNA analogue (Inoue et al., 1987).

The antisense molecules should be delivered to cells which express the target allele. A number of methods have been developed for delivering antisense DNA or RNA to cells; e.g., antisense molecules can be injected directly into a tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore a preferred approach utilizes a recombinant DNA construct in which the antisense nucleic acid is placed under the control of a strong promoter. The use of such a construct to transfect target cells will result in the transcription of sufficient amounts of single stranded RNAs that will form complementary base pairs with the endogenous target allele transcripts and thereby prevent translation of the target allele mRNA. For example, a vector can be introduced such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981), the promoter contained in the 3′ long terminal repeat of Rous sarcoma virus (Yamamoto et al., 1980), the herpes thymidine kinase promoter (Wagner et al., 1981), the regulatory sequences of the metallothionein gene (Brinster et al, 1982), the rhodopsin promoter (McNally et al. 1999; Zack et al., 1991), the collagen 1A2 promoter (Akai et al. 1999; Antoniv et al. 2001), the collagen 1A1 promoter (Sokolov et al. 1995; Breault et al. 1997) and others. Any type of plasmid, cosmid, YAC or viral vector can be used to prepare the recombinant DNA construct which can be introduced directly into the tissue site; e.g., the bone marrow. Alternatively, viral vectors can be used which selectively infect the desired tissue; (e.g., for brain, herpesvirus vectors may be used), in which case administration may be accomplished by another route (e.g., systemically).

The antisense constructs of the present invention, by antagonizing the normal biological activity of the target allele proteins, can be used in the modulation (i.e., activation or stimulation, e.g., by agonizing or potentiating and inhibition or suppression, e.g., by antagonizing, decreasing or inhibiting) of cellular activity both in vivo and, likewise, for ex vivo tissue cultures.

The antisense techniques can be used to investigate the role of target allele RNA or protein product in developmental events, as well as the normal cellular function of target allele products in adult tissue. Such techniques can be utilized in cell culture, but can also be used in the creation of transgenic animals.

### Triple Helix Suppressor

Endogenous target allele gene expression can be reduced by targeting DNA sequences complementary to the regulatory region of the target allele (i.e., the target allele promoter and/or enhancers) to form triple helical structures that prevent transcription of the target allele in target cells in the body (Helene, 1991; Helene et al., 1992; Maher, 1992).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex. Alternatively, other suppression effectors such as double stranded RNA could be used for suppression.

Antisense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al., 1988, methylphosphonate oligonucleotides can be prepared by use of controlled pore glass polymer supports (Sarin et al., 1988).

Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stablely into cell lines.

### Hammerhead Ribozymes of the Invention

The invention provides for suppression of a mutant gene in a mutation-independent manner by targeting a polymorphism in a gene and providing, if necessary, a replacement gene which is a different polymorphic variant, and that is therefore protected either partially or completely from suppression. The use of a mutation-independent suppression approach exploiting polymorphism enables much of the mutational heterogeneity inherent in diseases such as OI to be circumvented. Many different agents may be used to suppress gene expression such as, for example, antisense RNA and DNA, triple helix DNA, double stranded RNA, peptide nucleic acids, ribozymes and so on.

The invention is demonstrated herein by hammerhead ribozymes. Detailed kinetic analysis of the cleavage profile of a hammerhead ribozyme under single and multiple turnover kinetics provides a clear indication of its efficiency in vitro. While kinetic profiles obtained for ribozymes in vitro do not accurately represent the cleavage efficiency that may be achieved in vivo, such studies can provide a broad indication as to potential in vivo efficiencies (Birikh et al., 1997). For this reason a detailed kinetic profile for a hammerhead ribozyme targeting the COL1A1 transcript has been developed and is presented below in Example 2.

Hammerhead ribozyme RzPolCol1A1 has its cleavage site at a common (2 pq=0.4; Millington-Ward et al., 1999) intragenic polymorphic site (CUC3210CCC; Weterhausen et al., 1990) enabling generation of a replacement gene with the alternate polymorphic sequence but which nevertheless codes for wild-type or functional COL1A1 protein. Transcripts from replacement genes escape ribozyme cleavage due to the absence of the ribozyme cleavage site and therefore can be translated to provide wild-type or functional protein. A detailed kinetic profile of RzPolCol1A1 targeting the human COL1A1 transcript is provided.

### Ribozyme Kinetics

Kinetic profiles of ribozymes in vitro can be used as broad predictors of potential efficiencies in vivo (Birikh et al., 1997). As the cellular milieu has been demonstrated in some cases not to differentially alter the activity of ribozymes tested in vitro when used in vivo (Chowrira et al., 1994), it is instructive to assess kinetic behaviour of ribozymes by developing kinetic models of catalytic activity in vitro prior to evaluation in cell-culture, animal or plant systems. This may aid in prioritizing which of a battery of ribozymes should be evaluated initially in vivo. Ribozymes, like their protein counterparts, may follow Michaelis-Menten kinetic regimes (Fedor and Uhlenbeck, 1992; Hendry et al., 1997). A kinetic description for one turnover of a hammerhead ribozyme reaction involves (a) binding of the ribozyme and substrate to form a ribozyme-substrate complex, (b) cleavage of the phosphodiester backbone of substrate RNA to produce ribozyme bound 5′ and 3′ products and (c) two pathways for dissociation of product RNA thereby releasing free ribozyme for rebinding to intact substrate (Hertel et al., 1994).

### Kinetic Pathway for a Hammerhead Ribozyme

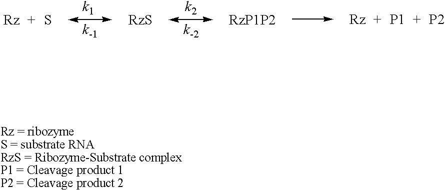

Each step in this pathway, in both forward and reverse direction, is defined by a measurable elemental rate constant (k₁, k₂, k₋₁, k₋₂). Therefore, by varying the relative molar concentrations of ribozyme and substrate under suitable experimental conditions (typically 10 mM MgCl₂, pH 7.5 and 37° C.), the catalytic pathway for the ribozyme can be evaluated into individual components and the kinetic constant corresponding to each step determined. For instance, single-turnover experiments are carried out in conditions where a saturating excess of ribozyme has bound to the substrate. Experimentally, by preannealing large excesses of radioactively labelled ribozyme to substrate, all substrate molecules become bound by ribozyme. The cleavage reaction is then initiated by the addition of MgCl₂ and the half-life of substrate RNA, t_{1/2}, and the rate constant for the cleavage reaction, k₂, can be directly measured (see section on methods and results for details).

In contrast to single-turnover kinetics, multiple-turnover cleavage reactions may be carried out to determine the ribozyme's ability to function as a true re-cycling catalyst. In this case, reactions are performed under conditions of substrate excess and as ribozymes follow Michaelis-Menten kinetic regimes (Fedor and Uhlenbeck, 1992), kinetic parameters such as the maximum rate of cleavage (Vₘₐₓ) and the Michaelis constant (Kₘ) can be determined. Kₘ resembles a dissociation constant in that it has dimensions of concentration and specifies the relative concentration of free enzyme, free substrate and enzyme-substrate complex under steady-state conditions. In practical terms, it may be viewed as a measure of the affinity of ribozyme for substrate RNA (Cornish-Bowden and Wharton, 1990).

### Mesenchymal Progenitor Stem Cells—a Model System for OI Therapy

A possible route of gene therapy for OI may involve removal of cells expressing COL1A1 or cells which are the progenitors of COL1A1 expressing cells from bone marrow of a patient, ex vivo genetic manipulation of these cells and subsequent re-administration of manipulated cells into the patient. Target cells for such a procedure in relation to OI are amongst others embryonic stem cells, blood mononuclear cells (MNCS) (See U.S. Pat. No. 5,399,346, which is incorporated herein by reference) and mesenchymal progenitor stem cells (MPCs) (See U.S. Pat. Nos. 5,486,359; 5,811,094; 6,010,696; 6,030,836 for example, which are incorporated herein by reference). These undifferentiated cells are present in bone marrow and give rise to osteoblasts, the cell type implicated in OI (Pittenger et al., 1999) as well as other cell types. These cells are commercially available from BioWhittaker and are known to be susceptible to retroviral (Allay et al., 1997) and adenoviral infection (Conget and Minguell, 2000). In addition, donor mesenchymal progenitor stem cells have been successfully transplanted into the bone marrow of children with OI (Horwitz et al., 1999). Thus mesenchymal progenitor stem cells are a useful vehicle for delivery of therapeutic genes to bone marrow of OI patients. An exemplary procedure is provided in Example 8.

Gene Suppressor Delivery

Many in vitro studies have been undertaken on methods of gene suppression using, for example, antisense DNA or RNA, double stranded RNA, PNAs and various types of ribozymes. Efficient gene suppressors are then studied either in cell culture or animal models, for example, there are two generally accepted methods of introducing ribozymes into cells. The first is through exogenous delivery of pre-formed ribozymes and the second through endogenous delivery, which involves the expression of ribozymes from transcriptional units such as DNA plasmids. Endogenous expression has been achieved on many occasions, using plasmids and viruses with strong promoters driving ribozyme expression, such as the SV40, CMV, RNA polymerase II (pol II) and beta-actin, promoters or tissue specific promoters such as the COL1A or rhodopsin promoter. To aid delivery of plasmids into target cells, plasmids can be associated with non-viral vectors such as lipids and synthetic polymers.

Complete silencing of a disease allele may be difficult to achieve using antisense, PNA, ribozyme and triple helix approaches or any combination of these. However, small quantities of mutant gene product may be tolerated in some disorders. In others, a significant reduction in the proportion of a mutant gene product to normal product may result in an amelioration of disease symptoms. Hence, this strategy may be applied to any dominant or polygenic disease in animals where the molecular basis of the disease has been established. In addition, the strategy is applicable to modulating infectious disorders. This strategy will enable the same therapy to be used to treat a range of different disease mutations within the same gene. The term “treat” as used herein is intended to encompass curing as well as ameliorating at least one symptom of the condition or disease. Likewise, a “therapeutic” is an agent which cures or ameliorates at least one symptom of the condition or disease. The invention provides important therapies for dominant and polygenic diseases, that circumvent the need for a specific therapy for every mutation causing or predisposing to a disease. This is particularly relevant in some disorders, for example, rhodopsin linked autosomal dominant RP, in which to date about one hundred different mutations in the rhodopsin gene have been observed in adRP patients. Likewise hundreds of mutations have been identified in the human type I collagen 1A1 and 1A2 genes in autosomal dominant osteogenesis imperfecta. Costs of developing therapies for each mutation are prohibitive at present. The present invention uses a general approach for treating cells with dominant-negative mutations which cause or predispose to disease pathology or for therapy for such diseases. Such general approaches may target the primary defect as described in the exemplification. In addition, the methods of the invention can target secondary effects such as, for example, apoptosis. Polymorphism has been proposed as a method for suppressing one allele of a gene(s) whose product(s) is vital to cell viability. This has been proposed particularly in relation to treatment of tumors where one allele is absent in tumor cells and therefore suppression of the second allele, which is vital for cell viability, may result in induction of tumor cell death. Non-tumorous diploid cells should, in principle, remain viable as they should still maintain one functioning wild-type allele, even after the suppression therapy has been administered (D. E. Housman, PCT/US94/08473).

The invention may be applied in gene therapy approaches for biologically important polygenic disorders affecting large proportions of the world's populations, such as age-related macular degeneration, glaucoma, manic depression, cancers having a familial component and indeed many others. Polygenic diseases require inheritance of more than one mutation or component to give rise to the disease state. Notably an amelioration in disease symptoms may require reduction in the presence of only one of these components, that is, suppression of one mutant genotype, which together with others leads to the disease phenotype, may be sufficient to prevent or ameliorate symptoms of the disease. In some cases suppression of more than one component may be required to improve disease symptoms. This invention thus provides polygenic interventive therapies for common polygenic diseases to suppress a particular genotype(s) associated with one or more polymorphisms, thereby suppressing the disease phenotype.

Practice of the invention will be still more fully understood from the following examples, which are presented herein for illustration only and should not be construed as limiting the invention in any way. Variations and alternate embodiments will be apparent to those of skill in the art. The contents of all cited references (including literature references, issued patents, published patent applications that may be cited throughout this application) are hereby expressly incorporated by reference. The practice of the present invention will employ, unless otherwise indicated, conventional techniques of molecular biology, cell biology, cell culture, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al., U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al., eds.), the entire contents of which are hereby incorporated by reference.

### EXEMPLIFICATION

The present invention is exemplified using three genes: human rhodopsin and human collagen 1A1 and 1A2. The first of these genes is retina specific. In contrast, collagens 1A1 and 1A2 are expressed in a range of tissues including skin and bone. While these three genes have been used as examples, there is no reason why the invention could not be deployed for the suppression of individual allelic variants of many other genes in which mutations cause or predispose to a deleterious effect. Many examples of mutant genes which give rise to disease phenotypes are available from the prior art. Similarly, many polymorphisms have been identified in genes in which disease causing or predisposing mutations have been observed—these genes all represent targets for the invention. Some exemplary polymorphisms for use as target alleles of the invention are given in Tables 5-11.

The present invention is exemplified using hammerhead ribozymes with antisense arms to elicit RNA cleavage. There is no reason why other suppression effectors such as antisense RNA, antisense DNA, triple helix forming oligonucleotides, PNAs and peptides, directed towards individual polymorphic variants of genes or gene products could not be used to achieve gene suppression. Hammerhead ribozymes with antisense arms were used to elicit sequence specific cleavage of RNA transcribed from a vector containing one allele of a polymorphism and non-cleavage of RNA from a vector containing a different allelic variant of the polymorphism. Uncleavable alleles could be used as a replacement nucleic acid, if required, to restore levels of wild-type protein, thereby preventing pathology due to haplo-insufficiency.

In Example 1, ribozymes were designed to cleave alleles at a single polymorphic site. In one example, collagen 1A2, two ribozymes were used to target two different polymorphic sites located 6 bases apart, often in the same open loop structure in the predicted 2-D conformations of the collagen 1A2 transcript. One ribozyme targets one allele of the first polymorphism in collagen 1A2 while the second ribozyme targets one allele of the second polymorphism in collagen 1A2. If necessary, multiple polymorphisms within or close to a gene in the same allele could be targeted to achieve efficient and specific suppression of an individual allele. For example, naturally occurring polymorphic variants have been observed in the retinal specific genes encoding the photoreceptor proteins rhodopsin and peripherin (Table 1). Although these do not occur at appropriate ribozyme cleavage sites (i.e., NUX sites in RNA open loop structures) approaches such as, for example, antisense, triplex helix or antibodies could be utilized to achieve suppression of single alleles carrying disease mutations while enabling continued expression from alternative allelic forms of the gene with wild-type sequence, using these or other polymorphisms. Further sequencing of the introns of these retinal genes may reveal other appropriate polymorphic target sites for ribozymes. Furthermore and artificially created polymorphism in the rhodopsin gene has been used to demonstrated the invention. Such artificial polymorphisms may be used alone or in concert with naturally occurring polymorphisms to minimize suppression in replacement genes.

The invention is exemplified using suppression effectors directed to target single allelic variants of human rhodopsin and human collagens 1A1 and 1A2 genes or transcripts, targeting polymorphic sites in coding or 3′ untranslated regions of their transcripts. There is no reason why polymorphisms in other transcribed but untranslated regions of genes or in introns or in regions involved in the control of gene expression, such as promoter regions, or in regions adjacent to a gene or any combination of these could not be used to achieve gene suppression. Suppression targeted to any polymorphism within or close to a gene may allow selective suppression of one allele of the gene carrying a deleterious mutation while maintaining expression of the other allele. Multiple suppression effectors, for example shotgun ribozymes, could be used to optimise efficiency of suppression if necessary. Additionally, when required, expression of a replacement nucleic acid comprising an allelic variant sequence different from the sequence to which suppression effector(s) are targeted may be used to restore levels of wild-type gene product.

Human rhodopsin and human collagen 1A1 and 1A2 cDNA clones representing specific polymorphic variants of these genes were transcribed in vitro. Ribozymes targeting specific alleles of the human rhodopsin and collagen 1A1 and 1A2 cDNAs were also expressed in vitro. cDNA clones were cut with various restriction enzymes resulting in the production of differently sized transcripts after expression. This aided in differentiating between RNAs expressed from cDNAs representing different alleles of polymorphisms in rhodopsin and collagen 1A1 and 1A2. Restriction enzymes used to cut each clone, sizes of resulting transcripts and predicted sizes of products after cleavage by target ribozymes are given below in Table 3. Exact sizes of expression products may vary by a few bases from that estimated, as there is some ambiguity about the specific base at which transcription starts (using the T7 promoter) in pcDNA3.

### Example 1

### Cleavage of Wild-Type (Unmutated) and Mutated Rhodopsin and collagen 1A1 and 1A2 RNA by Ribozymes

cDNA templates and ribozyme DNA fragments were cloned into commercial expression vectors (pcDNA3, Invitrogen; pZeoSV, Invitrogen; or pBluescript, Stratagene) which enabled expression in vitro from T7, T3 or SP6 promoters or expression in cells from CMV or SV40 promoters. DNA inserts were placed into the multiple cloning site (MCS) of these vectors typically at or near the terminal ends of the MCS to delete most of the MCS and thereby prevent any possible problems with efficiency of expression subsequent to cloning. Clones containing template cDNAs and ribozymes were sequenced by an automated sequencing machine (Applied BioSystems, 373A DNA Sequence), using standard protocols.

RNA was obtained from clones in vitro using a commercially available Ribomax expression system (Promega) and standard protocols. RNA purifications were undertaken using the Bio-101 RNA purification kit or a solution of 0.3M sodium acetate and 0.2% SDS after running on 4%-8% polyacrylamide gels. Cleavage reactions were performed using approximately 30-80 nM target RNA and over up to 1000-fold more ribozyme, using standard ribozyme protocols with 5 mM Tris-HCl pH8.0 and varying MgCl₂ concentrations (0-15 mM) at 37° C., typically for 3 hours. Time points were performed at the predetermined optimal MgCl₂ concentrations for up to 5 hours. Radioactively labeled RNA products were obtained by incorporating α-P³² rUTP (Amersham) in the expression reactions (Gaughan et al., 1995). Labeled RNA products were run on polyacrylamide gels before cleavage reactions were undertaken for the purposes of RNA purification and subsequent to cleavage reactions to establish if RNA cleavage had been achieved.

Predictions of the secondary structures of human rhodopsin and human collagens 1A1 and 1A2 mRNAs were obtained using the RNAPlotFold program (Wisconsin Package, Version 10.0, Genetics Computer Group (GCG), Madison Wis.). Though the program can predict likely secondary structures for a given RNA target sequence, it cannot absolutely predict the likelihood of success or failure of a ribozyme directed at a certain region in that RNA molecule. Ribozymes and antisense were designed to target areas of the RNA that were predicted to be accessible to suppression effectors according to criteria known those skilled in the art of RNA structure or ribozymes. The integrity and robustness of open loop structures was evaluated from the 10 most probable RNA structures. Additionally, RNA structures for truncated RNA products were generated and the integrity of open loops between full length and truncated RNAs was compared. RNA structures for 6 mutant rhodopsin transcripts were generated and the open loop structures targeted by ribozymes compared between mutant transcripts (Table 1).

The human rhodopsin cDNA (SEQ ID NO: 1) was cloned into the HindIII and EcoRI sites of the MCS of pcDNA3 in a 5′ to 3′ orientation allowing subsequent expression of RNA from the T7 or CMV promoters in the vector. The full length rhodopsin 5′UTR sequence was inserted into this clone using primer driven PCR mutagenesis and a HindIII (in pcDNA3) to BstEII (in the coding sequence of the human rhodopsin cDNA) DNA fragment.

Briefly, PCR directed mutagenesis was performed as follows. The general protocol involves three PCR reactions (PCR1, PCR2 and PCR3). In the case of mutagenizing rhodopsin for example, PCR1 amplifies the sequence between the HindIII site (5′ in the MCS of the vector pcDNA3) and the site where one desires to introduce a mutation. The reverse primer has a base change at the site where the mutation is being introduced, so that PCR1 products contain an altered base. PCR2 amplifies the DNA between the site of mutagenesis and the BstEII site which is present in the rhodopsin cDNA. However, the forward primer for this PCR has a sequence alteration at the site where the mutation is being introduced. PCR2 products therefore also contain an altered base at this site. The PCR1 and PCR2 were run on a 1% agarose gel and the amplification products were isolated from the gel using columns such as Genelute Agarose Spin columns (Sigma) or an Agarose Gel Gene Extraction kit (Boerhinger Mannheim). The resulting DNA amplified products are used in a third PCR reaction which amplified the whole region between the HindIII and BstEII sites. Resulting amplifications contained the altered bases. Amplification product were digested with HindIII and BstEII and cloned into the HindIII and BstEII sites of a pcDNA3 clone containing the full length rhodopsin cDNA which was also digested with HindIII and BstEII. Table 2 lists the primers used for mutagenesis in Example 1.

TABLE 2

| | |
|---|---|
| Primer | SEQ ID NO |

Rhodopsin (5′ > 3′)

| | | |
|---|---|---|
| Forward over HindIII site | ACCCAAGCTTAGAGTCATCC | 2 |
| Reverse over BstEII site | ACCATGCGGGTGACCTCCTT | 3 |
| Forward over mutation site | ATCGTGCTGGTCTTCCCCAT | 4 |
| Reverse over mutation site | ATGGGGAAGACCAGCACGAT | 5 |

Collagen 1A2 (5′ > 3′)

| | | |
|---|---|---|
| Forward | CAGAGATGGTGAAGATGGT | 6 |
| Reverse | AGTCCTCTGGCACCAGTAGC | 7 |
| Forward mutagenesis | TAACGCTGGTCCTACTGGACCCG | 8 |
| Reverse mutagenesis | CGGGTCCAGTAGGACCAGCGTTA | 9 |

Collagen 1A1 (5′ > 3′)

| | | |
|---|---|---|
| Forward | AGTCACACCGGAGCCTGGGG | 10 |
| Reverse | GGTAAGGTTGAATGCACTTTTG | 11 |

In the case of collagen 1A1, no mutagenesis was carried out as both versions of the 3210 polymorphic site (3210C and 3210T) were found in human DNA samples from CEPH (Centre d'Etude du Polymorphisme Humain (CEPH) in Paris; Dausset et al. (1990) Genomics 6:575-577) panel DNA. Twelve random DNAs from unrelated individuals from the CEPH panel where sequenced over the 3210 polymorphic site and two DNA samples with T/T and C/C genotypes identified. A small region of 371 bp over the polymorphic site was amplified from these people using primers in Table 2 with SEQ ID NO: 10 and SEQ ID NO: 11 and cloned from each of the samples.

The human rhodopsin hybrid cDNA with a single base alteration, (a C-->G change at position 477) was introduced into human rhodopsin cDNA using a HindIII to BstEII PCR cassette by primer directed PCR mutagenesis according to the standard method described above. Primers used for mutagenesis were the following (Table 2): PCR1 used SEQ ID NO:2 and SEQ ID NO:5, PCR2 used SEQ ID NO:4 and SEQ ID NO:3 and PCR3 used SEQ ID NO:2 and SEQ ID NO:3. This artificial polymorphism occurs at a silent position, i.e., it does not give rise to an amino acid substitution, however the C>G change eliminates the ribozyme cleavage site (i.e., GUX-->GUG). The hybrid rhodopsin (also referred herein as “mutated rhodopsin”) (SEQ ID NO: 12) was cloned into pcDNA3 in a 5′ to 3′ orientation allowing subsequent expression of RNA from the T7 or CMV promoters in the vector.

A human rhodopsin adRP mutation, a single base alteration (a C-->T change at codon 23) was introduced into human rhodopsin cDNA using a HindIII to BstEII PCR cassette by primer directed PCR mutagenesis according to the standard method described above. This sequence change results in the substitution of a leucine for a proline residue (“Pro23Leu”) (position 88 in sequence listing). Additionally, the nucleotide change (CCC-->CTC) creates a ribozyme cleavage site. The mutated rhodopsin (SEQ ID NO: 13) was cloned into the HindIII and EcoRI sites of pcDNA3 in a 5′ to 3′ orientation allowing subsequent expression of RNA from the T7 or CMV promoters in the vector.

A hammerhead ribozyme (termed Rz10) (SEQ ID NO: 14), designed to target a large open loop structure in the RNA from the coding regions of the wild-type gene, was synthesized, annealed and cloned into the HindIII and XbaI sites of pcDNA3, allowing expression of RNA from the T7 or CMV promoters in the vector. NB: There is a one base mismatch in one of the antisense arms of Rz10. The target site was GUC (the NUX rule) at position 322-324 of SEQ ID NO: 1. A hammerhead ribozyme (termed Rz20) (SEQ ID NO: 15), designed to target an open loop structure in RNA from the coding region of a rhodopsin gene with a Pro23Leu mutation, was synthesized, annealed and cloned into the HindIII and XbaI sites of pcDNA3 again allowing expression of RNA from the T7 or CMV promoters in the vector. The target site was CTC (the NUX rule) at codon 23 of the human rhodopsin sequence (position 88 of SEQ ID NO: 13) (Accession number: K02281). The antisense flanking sequence of Rz10 and Rz20 are underlined.

(SEQ ID NO:14)

Rz10: GGTCGGTCTGATGAGTCCGTGAGGACGAAACGTAGAG

(SEQ ID NO:15)

Rz20: TACTCGAACTGATGAGTCCGTGAGGACGAAAGGCTGC

Briefly, ribozyme cDNAs were prepared as follows. Sense and antisense ribozyme oligonucleotides were synthesized by commercial companies (either VHBio or Genesys). Ribozyme oligonucleotides were designed and synthesized to contain a HindIII site at the 5′ end of the ribozyme and an XbaI site at the 3′ end of the ribozyme. Equal amounts of the sense and antisense ribozyme oligonucleotides were combined in an eppendorf tube with 1 μl of 1 M NaCl. H₂O was added to a final volume of 40 μl. The eppendorf tube was heated to 94° C. for 3 minutes and left overnight at room temperature to anneal the sense strand to the antisense strand. Annealed ribozyme was digested with HindIII and XbaI and cloned into the HindIII and XbaI sites of expression vector pcDNA3 (Invitrogen) using a ligation kit (Stratagene) and the manufacturers protocols. Ligations were transformed into XL1 blue MRA cells using standard protocols in the art. Resulting clones were selected with Ampicillin, picked and grown in bulk using standard procedures. Plasmid DNA was isolated using standard procedures and sequenced in an automated sequencing machine (ABI).

A section of the human collagen 1A1 cDNA was cloned from genomic DNA from two individuals with the T/T and the C/C genotype into the HindIII and XbaI sites of pcDNA3 according to the standard method described above (Table 2). The clones were in a 5′ to 3′ orientation allowing subsequent expression of RNA from the T7 or CMV promoters in the vector. The clones contain the collagen 1A1 sequence from position 2977 to 3347 (Accession number: K01228) (2977 to 3347 of SEQ ID NO:16). Clones containing allele A (position 233 of SEQ ID NO: 17) and allele B (nucleotide 233 of SEQ ID NO: 18) of a naturally occurring polymorphism in the 3′UTR (Westerhausen et al., 1990) were identified by sequence analysis according to standard methods. Allele A has a T at position 3210 (position 233 of SEQ ID NO: 17) and allele B has a C at position 3210 (position 233 of SEQ ID NO:18).

A hammerhead ribozyme (termed RzPolCol1A1) (SEQ ID NO:19) designed to target a large open loop structure (as determined from the ten most probable 2-D structures) in the RNA from the 3′UTR of the type I collagen 1A1 gene was synthesized and cloned into the Hind III and XbaI sites of pcDNA3 according to the standard method described above, again allowing subsequent expression of RNA from the T7 or CMV promoters in the vector. The ribozyme target site was a GUX site at position 3209-3211 of the human collagen 1A1 sequence (Accession number: K01228) (nucleotide 3209 to 3211 of SEQ ID NO:16). The antisense flanking sequences of RzPolCol1A1 are underlined.

(SEQ ID NO:19)

RzPo1Co11A1: TGGCTTTTCTGATGAGTCCGTGAGGACGAAAGGGGGT

A human type I collagen 1A2 cDNA was obtained from the ATCC (Accession No: Y00724) (SEQ ID NO:20). Two naturally occurring polymorphisms have previously been found in collagen 1A2, one at position 902 involving a G-->A and one at 907 involving a T-->A nucleotide change, respectively (Filie et al., 1993). Both polymorphisms occur often in the same predicted open loop structure of human collagen 1A2 RNA. Polymorphic variants of human collagen 1A2 were generated by PCR directed mutagenesis using a HindIII to XbaI PCR cassette according to the standard method described above (Table 2). Primers used for PCR1 were SEQ ID NO:6 and SEQ ID NO:9. Primers used for PCR2 were SEQ ID NO:8 and SEQ ID NO:7 and primers used for PCR3 were SEQ ID NO:6 and SEQ ID NO:7 (Table 2). Resulting clones contained the following polymorphisms: collagen 1A2 (A) has a G nucleotide at position 902 and a T nucleotide at position 907 (nucleotide 674 and 679 of SEQ ID NO:21, respectively). In contrast, human collagen 1A2 (B) has A nucleotides at both positions 902 and 907 (nucleotide 674 and 679 of SEQ ID NO:22). The site at 902 creates a ribozyme target site in collagen 1A2 (B), that is an NUX site (900-902), but is not a ribozyme target site in collagen 1A2 (A), in that it breaks the NUX rule—it has a G nucleotide in the X position. In contrast, in collagen 1A2 (A) there is a ribozyme target site at position 907, that is a GTC site (906-908) however this site is lost in collagen 1A2 (B) the sequence is altered to GAC (906-908), thereby disrupting the ribozyme target site.

Hammerhead ribozymes (termed Rz902 and Rz907) were designed to target a predicted open loop structures in the RNA from the coding region of polymorphic variants of the human collagen 1A2 gene. Rz902 (SEQ ID NO:23) and Rz907 (SEQ ID NO:24) oligonucleotides were synthesized, annealed and cloned into the HindIII and XbaI sites of pcDNA3, allowing subsequent expression of RNA from the T7 or CMV promoters in the vector. The target sites were NUX sites at positions 900-902 and 906-908 of the human type I collagen 1A2 sequence (Accession number: Y00724), respectively. The antisense flanking sequences of Rz902 and Rz907 are underlined.

(SEQ ID NO:23)

Rz902: GGTCCAGCTGATGAGTCCGTGAGGACGAAAGGACCA

(SEQ ID NO:24)

Rz907: CGGCGGCTGATGAGTCCGTGAGGACGAAACCAGCA

The plasmid pBR322 was cut with MspI, radioactively labeled and run on a polyacrylamide gel to enable separation of the resulting DNA fragments. This digestion gave DNA sizes of 76, 90, 110, 123, 147, 160, 180, 190, 201, 217, 238, 242, 307, 404, 527 and 622 base pairs. This DNA ladder was then used on subsequent polyacrylamide gels (4-8%) to provide an estimate of the size of the RNA products run on the gels. Though the mobility of DNA and RNA of the same length may vary depending on the percentage of polyacrylamide and the gel running conditions, the markers provide an estimate of the size of transcripts.

The unmutated wild-type human rhodopsin cDNA was expressed by in vitro transcription from the T7 promoter to the BstEII site in the rhodopsin coding sequence. Resulting RNA was mixed with Rz10 RNA in 15 mM magnesium chloride and incubated at 37° C. for 0, 1, 2 and 3 hours. Sizes of the expressed RNAs and cleavage products were as predicted (Table 3). Complete cleavage of human rhodopsin RNA was obtained, with a less than 5% residual amount of intact RNA present at 1 hour.

The mutated human rhodopsin cDNA was expressed from the T7 promoter to the BstEII site in the coding sequence. Resulting RNA was mixed with Rz10 and 0, 5, 10 and 15 mM magnesium chloride and incubated at 37° C. for 3 hours. The mutated rhodopsin transcripts with an artificial polymorphism were not cleaved by Rz10. Cleavage of mutated transcripts would have resulted in cleavage products of 564 bases and 287 bases, which were not present on the gel.

The unmutated human rhodopsin cDNA was expressed from the T7 promoter to the FspI site in the coding sequence. The mutated human rhodopsin cDNA was expressed from the T7 promoter to the BstEII site in the coding sequence. Both rhodopsin RNAs were mixed together with Rz10 and 15 mM magnesium chloride and incubated at 37° C. for 0, 1, 2 and 3 hours. The smaller unmutated rhodopsin transcripts were cleaved by Rz10 while the larger mutated transcripts were not cleaved (i.e., were protected from cleavage) by Rz10. Cleavage of mutated protected transcripts would have resulted in products of 564 bases and 287 bases. The 564 base product was not present. The 287 base product is also generated by cleavage of the unmutated human rhodopsin transcripts and hence is present. After 3 hours over 90% of the unmutated rhodopsin transcripts was cleaved by Rz10.

Unmutated and mutated human rhodopsin cDNAs were expressed from the T7 promoter to the Acyl site downstream of the coding sequence and the BstEII site in the coding sequence, respectively. Sizes of expressed RNAs and cleavage products were as predicted (Table 3). Resulting RNAs were mixed together with Rz10 RNA 0, 5, 10 and 15 mM MgCl₂ and incubated at 37° C. for 3 hours. Almost complete cleavage of the larger unmutated human rhodopsin RNA was obtained with a small residual amount of less than 10% of intact RNA present at 5 mM MgCl₂. In contrast, the mutated human rhodopsin RNA remained intact.

The mutant (Pro23Leu) human rhodopsin cDNA was expressed from the T7 promoter to the BstEII site in the coding sequence. Likewise, the Rz20 clone was expressed to the XbaI site. Resulting RNAs were mixed together with 5 mM magnesium chloride concentrations at 37° C. for 0, 0.5, 1, 2 and 5 hours. Sizes of expressed RNAs and cleavage products were as predicted (Table 3). Almost complete cleavage of mutant rhodopsin transcripts was obtained with a residual amount less than 5% of intact RNA left even after 5 hours.

The mutant (Pro23Leu) human rhodopsin cDNA was expressed from the T7 promoter to the BstEII site in the coding sequence. Likewise, the Rz10 clone was expressed to the XbaI site. Resulting RNAs were mixed together in 10 mM magnesium chloride at 37° C. for 0, 0.5, 1, 2 and 5 hours. Sizes of expressed RNAs and cleavage products were as predicted (Table 3). Almost complete cleavage of mutant rhodopsin RNA was obtained with a residual amount less than 5% of intact RNA remaining even after 5 hours.

The human collagen 1A1 cDNA containing the T allele of the polymorphism at 3210 was expressed from the T7 promoter to the XbaI site in the vector. Resulting RNA was mixed together with RzPolCol1A1 in 0, 5, 10, 15 mM MgCl₂ and incubated at 37° C. for 3 hours. RNA transcripts were cleaved efficiently by RzPolCol1A1—a residual amount less than 2% of RNA remained at 5 mM MgCl₂.

The human collagen 1A1 cDNA containing the C allele of the polymorphism at position 3210 was expressed from the T7 promoter to the XbaI site in the vector. Resulting RNA was mixed together with RzPolCol1A1 in 0, 5, 10, 15 mM MgCl₂ and incubated at 37° C. for 3 hours. RNA transcripts were not cleaved by RzPolCol1A1—RNA remained intact over the range of MgCl₂ concentrations.

The human collagen 1A1 cDNA containing the T allele of the polymorphism at position 3210 was expressed from the T7 promoter to the XbaI site in the vector. Resulting RNA was mixed together with RzPolCol1A1 in 5 mM magnesium chloride and incubated at 37° C. for 0, 0.5, 1, 2, and 5 hours. Transcripts were cleaved by RzPolCol1A1 immediately upon addition of MgCl₂.

The human collagen 1A1 cDNA containing the C allele of the polymorphism at position 3210 was expressed from the T7 promoter to the XbaI site in the vector. Resulting RNA was mixed together with RzPolCol1A1 in 5 mM magnesium chloride and incubated at 37° C. for 0, 0.5, 1, 2, and 5 hours. RNA transcripts were not cleaved by RzPolCol1A1 even after 5 hours—no cleavage products were observed on the gel.

The human collagen 1A2 cDNAs containing the A (B clone) and T (A clone) alleles of the polymorphism at position 907 were expressed from the T7 promoter to the MvnI and XbaI sites in the insert and vector, respectively. Resulting RNAs were mixed together with Rz907 in 0, 5, and 10 mM MgCl₂ and incubated at 37° C. for 3 hours. RNA transcripts from the T allele containing the 907 target site were cleaved by Rz907 upon addition of divalent ions—cleavage was obtained at 10 mM MgCl₂ with a residual amount less than about 10% of transcript from the T allele remaining. In contrast, transcripts expressed from the A allele (which are smaller in size to distinguish between the A (MvnI) and T (XbaI) alleles) were not cleaved by Rz907— no cleavage products were observed.

The human collagen 1A2 cDNAs (B)+(A) clones containing the A and T alleles of the polymorphism at position 907, respectively, were expressed from the T7 promoter to the MvnI and XbaI sites in the insert and vector, respectively. Resulting RNAs were mixed together with Rz907 and 10 mM magnesium chloride and incubated at 37° C. for 0, 0.25, 0.5, 1, 2, and 5 hours. RNA transcripts from the T allele containing the position 907 target site were cleaved by Rz907— complete cleavage was obtained after 4 hours. In contrast transcripts expressed from the A allele (which are smaller in size to distinguish between the A (MvnI) and T (XbaI) alleles) were not cleaved by Rz907— no cleavage products were observed.

The human collagen 1A2 cDNA (A) and (B) clones containing the G and A alleles of the polymorphism at position 902, respectively, were expressed from the T7 promoter to the MvnI and XbaI sites in the insert and vector respectively. Resulting RNAs were mixed together with Rz902 in 0, 5, 10 and 15 mM MgCl₂ and incubated at 37° C. for 3 hours. RNA transcripts from the B clone containing the 902 target site are cleaved by Rz902 upon addition of divalent ions—the cleavage obtained with Rz902 is not very efficient (about 10% cleavage in all samples after 3 hours). In contrast transcripts expressed from the G allele (which are smaller in size to distinguish between the G (MvnI) and the A (XbaI) alleles) were not cleaved at all by Rz902— no cleavage products were observed.

TABLE 3

| | | Estimated | |
|---|---|---|---|
| | Restriction Enzyme | RNA Size | Cleavage Products |
| Human rhodopsin | BstEII | ~851 bases | 287 + 564 bases (Rz10) |
| | AcyI | ~1183 bases | 287 + 896 bases (Rz10) |
| | FspI | ~309 bases | 287 + 22 bases (Rz10) |
| Human rhodopsin | BstEII | ~851 bases | |
| artificial | | | |
| polymorphism | | | |
| Human rhodopsin | BstEII | ~851 bases | 170 + 681 (Rz20) |
| Pro-Leu | | | |
| Human rhodopsin | BstEII | ~851 bases | 287 + 564 (Rz10) |
| Pro-Leu | | | |
| Rz10 | XbaI | ~52 bases | |
| Rz20 | XbaI | ~52 bases | |
| Human Collagen | XbaI | ~381 bases | 245 + 136 bases |
| 1A1 (A) T Allele | | | (RzPolCol 1A1) |
| Human Collagen | XbaI | ~381 bases | |
| 1A1 (B) C Allele | | | |
| RzPolCol 1A1 | XbaI | ~52 bases | |
| Human Collagen | XbaI | ~888 bases | 689 + 199 bases (Rz907) |

| | |
|---|---|
| 1A2 (A) | G@902 |
| | T@907 |

| | | |
|---|---|---|
| Human Collagen | MvnI | ~837 bases |

| | |
|---|---|
| 1A2 (B) | A@902 |
| | A@907 |

| | | | |
|---|---|---|---|
| Human Collagen | MvnI | ~837 bases | |
| 1A2 (A) | | | |
| Human Collagen | XbaI | ~888 bases | 683 + 205 bases (Rz902) |
| 1A2 (B) | | | |
| Rz902 | XbaI | ~52 bases | |
| Rz907 | XbaI | ~52 bases | |

(RNA sizes are estimates)

### Example 2

### Kinetic Analysis of the RzPolCol1A1 Ribozyme

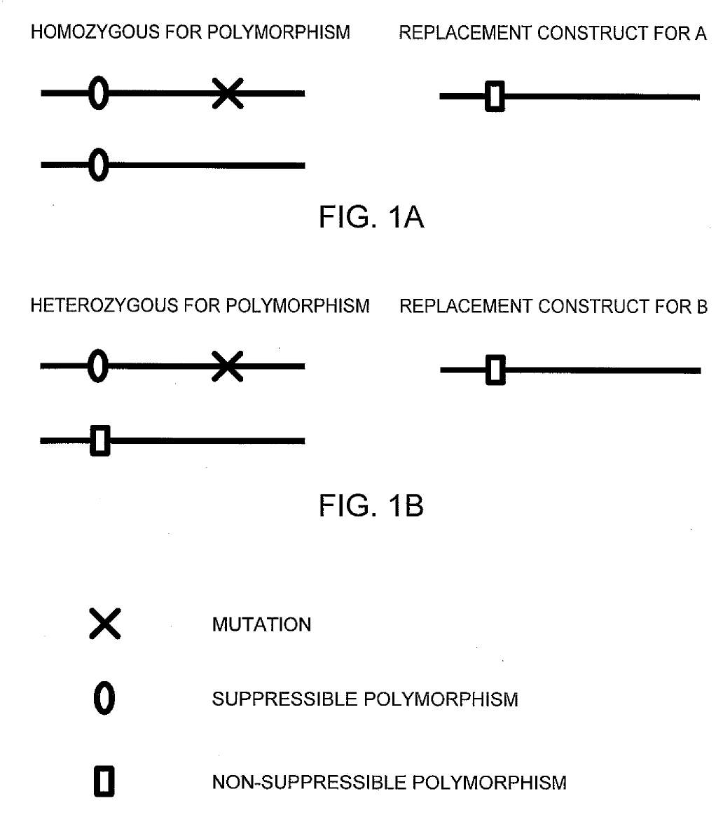

The embodiment exemplified in Example 1 relates to the specific suppression of alleles at polymorphic sites in genes with mutations which exert dominant negative or deleterious effect(s) (gain of function mutations). By not suppressing mutant alleles at mutation sites but at polymorphic sites some of the enormous genetic heterogeneity (situations where many different mutations within one gene giving rise to a similar disease pathology), associated with many diseases may be circumvented and the same method utilizing suppression and replacement used in cells of patients with different mutations in one gene. Thus a DNA or RNA sequence containing a known polymorphism that is on the same allele as one or more mutations in or around a gene is chosen as the target site of a suppression effector. The invention may be utilized as diagrammatically represented in FIGS. 1A and 1B.

In one embodiment, patients may be homozygous at a polymorphic site; that is, both the mutated allele and the wild-type allele have the same polymorphic variant at the site of suppression. Thus, the suppressor will suppress both the mutant and wild-type alleles. In rare instances, patients may be homozygous at a polymorphic site and also have the same mutation or different mutations present on both alleles in cells of such patients. In either case, suppression can be combined with a replacement step in which a replacement nucleic acid with an alternative polymorphic variant, which is not suppressed or is partially suppressed, is administered (FIG. 1A).

In another embodiment, patients may be heterozygous for a genetic polymorphism. In this situation the suppressing agent is targeted only to the polymorphic variant which is present on the same allele as the mutation. The suppressor will then selectively suppress the mutant allele at the polymorphic site and not suppress the wild-type allele, or suppress it less efficiently. To address situations where some disease pathology is associated with reductions in levels of wild-type protein (i.e., haploinsufficiency) a replacement gene with the polymorphic variant that is protected or partially protected from suppression will be administered (FIG. 1B). In some disorders the mechanism of disease pathology may be a function of both the reduction of wild-type protein and the presence of mutant protein. For example, in the case of OI, some collagen mutations cause a mild form of the disease due to haploinsufficiency (type I OI) while gain of function mutations result in more severe forms of the disease (types II-IV). Thus, in addition to suppressing a mutant allele, one may need to administer a wild-type allele to cells of OI patients.

The invention is exemplified below using a hammerhead ribozyme as a suppression agent. However, other suppressors such as antisense DNA or RNA, triple helix forming nucleic acids, double stranded RNA and PNAs may also be used. Though the exemplification provided herein is directed at OI, any mutant gene or genes with a dominant negative or deleterious effect, may be addressed using the allele suppression and replacement method of the invention.

The allele suppression and replacement methods of the invention have been exemplified with the hammerhead ribozyme, RzPolCol1A1, which targets the COL1A1 transcript at a common polymorphic site (T3210C). RzPolCol1A1 cleaves COL1A1 transcripts with a T at position 3210 (T-allele), but not COL1A1 transcripts with a C at position 3210 (C-allele). The 3210 polymorphism is extremely common; 2 pq=0.4 (p (T)=0.7; q (C)=0.3 (Millington-Ward et al., 1999). Therefore, it is estimated that 20% of OI patients will have the cleavable T at position 3210 of COL1A1 on the same allele as the mutation and a non-cleavable C on the other allele (FIG. 1A). In addition, 50% of OI patients will be homozygous for T at position 3210. It is therefore estimated that cells of about 70% of OI patients with dominant negative mutations in the COL1A1 gene could be treated with a single therapeutic agent (RzPolCOL1A1), and a replacement gene with a C at position 3210. The efficiency of T-allele cleavage by RzPolCol1A1 was assessed in vitro and RzPolCol1A1 was deemed to be highly efficient. In addition a method of protecting RzPolCol1A1 from nucleases was tested in vitro.

Oligonucleotides encoding the sense and antisense strands of RzPolCol1A1 were synthesized commercially as described above, purified, denatured and annealed in 25 mM NaCl overnight, according to standard methods as described in Example 1. Annealed primers were cut with XbaI and HindIII and were then cloned into the XbaI and HindIII sites of pcDNA3. The RzPolCol1A1 clone was cut with XbaI and transcribed in vitro using the T7 promoter in the vector with the incorporation of radioactively labeled ³²P αdUTP (Amersham). A 371 bp target Col1A1 transcript (positions 2977-3347) was cloned into pcDNA3 and transcribed in vitro. In vitro transcription products were gel purified and subsequently used in ribozyme cleavage reactions (Millington-Ward et al., 1997; Millington-Ward et al., 1999). Kinetic analysis was subsequently carried out to determine whether RzPolCol1A1 is a kinetically efficient ribozyme under both single- and multiple-turnover conditions (Millington-Ward et al., 1999).

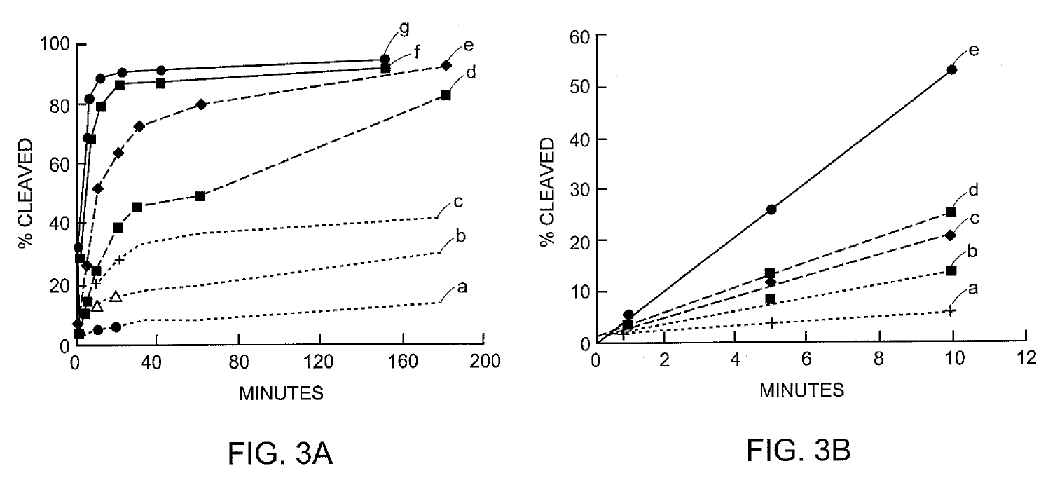

Prior to determining single-turnover parameters, it was necessary to identify the concentration of ribozyme required to achieve saturation. Under these conditions all molecules of substrate are bound by ribozyme, thus allowing an accurate quantification of t_{1/2} and k₂. Experimentally, substrate and ribozyme RNAs were combined in molar ratios ranging from 1:1-1:100 respectively. RNAs were heated at 90° C. for 3 minutes, cooled on ice and incubated at 37° C. in 50 mM Tris-HCl. Cleavage reactions were initiated with 10 mM MgCl₂ using standard protocols and as described herein. Aliquots were removed at various times, separated on 4-8% polyacrylamide gels and analyzed by radioactive instant imagery (Packard Instant Imager). Background was accounted for by analyzing equal areas of uncleaved and product RNAs. Ribozyme saturation rates were determined from graphs of percentage cleavage versus time (FIG. 3A). Notably, a ten fold molar excess of RzPolCol1A1 was sufficient to achieve saturation (FIG. 3A). Using a 100 fold molar excess of RzPolCol1A1 to T-allele RNA, the half-life of the substrate (t_{1/2}) and the rate of the cleavage step of the reaction (k₂) were determined. t_{1/2} was determined over 2 half lives from the initial rate of cleavage indicated by the slope of the graph of the fraction of uncleaved T-allele RNA versus time (FIG. 3A). k₂ was calculated from k₂=ln 2/t₁. Values of 1.2 min and 0.58 min⁻¹ were obtained for t_{1/2} and k₂ respectively (FIG. 3A, curve g).

In contrast to single-turnover reactions, multiple-turnover readings to measure Vₘₐₓ and Kₘ was taken under conditions of substrate excess within steady-state intervals. The steady-state interval is the time-frame in which cleavage reactions are proceeding most optimally and where no inhibition inherent to the reaction exists. The linear (steady-state) interval of T-allele cleavage by RzPolCol1A1 was determined by taking timepoints of cleavage reactions of varying ratios of substrate to ribozyme RNA. Plots of percentage cleavage versus time demonstrate that the reaction remains linear for up to at least 10 minutes (FIG. 3B). Subsequent cleavage reactions to measure Vₘₐₓ and Kₘ were performed at 8.5 minutes; a time deemed to be within the linear portion of the cleavage reaction. In these reactions, molar excesses of substrate to ribozyme RNA varied from 1.25:1-10:1. Vₘₐₓ and Kₘ were calculated using the following formulas (Cornish-Bowden and Wharton, 1990):

$${V}_{max} = \frac{\sum {v}^{2} / {a}^{2} \sum {v}^{2} - {( \sum {v}^{2} / a )}^{2}}{\sum {v}^{2} / {a}^{2} \sum v - \sum {v}^{2} / a \sum v / a}$$

$${K}_{m} = \frac{\sum {v}^{2} \sum v / a - \sum {v}^{2} / a \sum v}{\sum {v}^{2} / {a}^{2} \sum v - \sum {v}^{2} / a \sum v / a}$$

$$a = substrate concentration \\ v = rate of the reaction$$

RzPolCol1A1, despite its long target (371 bases) is very active, achieving a Vₘₐₓ of 0.4 min⁻¹. This is within the same order as commonly observed for many ribozymes directed to short unstructured RNAs (typically 20 bases, FIG. 3) which at maximum approximate ˜1 min⁻¹ (Zaug et al., 1988). Typically, reductions in Vₘₐₓ of 1000-fold or more have been observed for ribozymes directed to long structured RNAs (Heindenreich and Eckstein, 1992; Hendry et al, 1995; Janowsky and Schwenzer, 1996; Campbell et al, 1997). Such a reduction was not observed with RzPolCol1A1 targeting the 371 base human COL1A1 RNA. The Kₘ value for RzPolCol1A1 was 9.5 nM which may contribute to the ribozyme's high catalytic efficiency.

It is of note that RzPolCol1A1 is more active in vitro than many reported ribozymes (Stage-Zimmerman and Uhlenback, 1998). For example, one ribozyme which was significantly less active in vitro (Drenser et al., 1998) and targeting an artificially introduced site in the human rhodopsin gene provided strong in vivo suppression of the transcript when tested in a transgenic rat (Lewin et al., 1998). This study, while targeting an artificially generated ribozyme cleavage site (which is not present in wild-type human rhodopsin sequence) nevertheless demonstrated the potential utility of hammerhead ribozymes for gene suppression. Given efficient cleavage in vivo, RzPolCOL1A1 should prove valuable in the development of novel therapeutic approaches for autosomal dominantly inherited OI.

Ribozymes, such as RzPolCol1A1, that are highly efficient in vitro may, due to a short in vivo half-life, not be as efficient when they are delivered into cultured cells or animals. Thus, ribozymes which are delivered in an exogenous manner, are often protected from ribonucleases by the incorporation of a wide range of chemical modifications such as, the incorporation of phosphorothioates, 2′-aminonucleotides and 2′-O-alkyl groups, (Zhang et al., 1999; Pieken et al., 1991; Ludwig et al., 1998). The addition of 2′-aminonucleotides has the advantage of not only stabilizing ribozymes over 14,000-fold compared to unmodified versions (Sioud and Sorenson, 1998), but also of being easy to generate in vitro in the laboratory by simply adding protected nucleotides to transcription reactions.

For the reasons stated above RzPolCol1A1 was enzymatically generated with pyrimidines containing 2′-amino uridine and cytidine. Both the cleavage efficiency of protected RzPolCol1A1 and its stability in Dulbecco's modified eagle medium supplemented with 10% fetal calf serum (DMEM+) was tested in vitro. Protected and normal RzPolCol1A1 were incubated in DMEM+ for 0, 1 and 30 minutes and run on a polyacrylamide gel. After one minute, about half of the unprotected ribozyme had degraded, leaving no intact ribozyme after 30 minutes. The protected ribozyme, however, remained fully intact for at least 30 minutes. Due to the small quantity of protected RzPolCol1A1 generated, an unquantified amount of protected RzPolCol1A1 was added to timepoint (0, 30, 60 and 180 minutes) cleavage reactions of the T-allele RNA of COL1A1. Despite small quantities, protected RzPolCol1A1 cleaved the target transcript specifically. Other methods of protection such as the incorporation of phosphorothioates and 2′-O-allyl groups are also contemplated (Zhang et al, 1999; Ludwig et al., 1998).

### Example 3

### Methods for the Preparation and Handling of RNA

The source of RNA from which RNA molecules encoding proteins are isolated, is preferably obtained by in vitro transcription of template DNA. For example, the RNA can be synthesized in vitro from cDNA or genomic DNA. In a preferred embodiment of the invention, the DNA to be in vitro transcribed is operably linked to, and is located downstream of, an RNA polymerase promoter, e.g., a bacteriophage promoter, such as SP6, T3 or T7 promoter.

Many vectors for in vitro transcription are available commercially. These may contain one or more of the promoters SP6, T3 and T7 and may additionally contain a polyA sequence at the 3′ end of the polylinker in which the DNA of interest is inserted. A “polylinker” or “MCS” refers to a nucleotide sequence containing several restriction enzyme recognition sites. Examples of vectors include pcDNA3, the series of SP6 vectors, e.g., SP64 (Krieg and Melton, infra), BlueScript, and pCS2+. Vectors that can be used for in vitro transcription are also described, e.g., in U.S. Pat. No. 4,766,072.

An in vitro transcription reaction can be carried out according to methods well known in the art. Kits for performing in vitro transcription kits are also commercially available from several manufacturers. In an illustrative embodiment, an in vitro transcription reaction is carried out as follows. A vector containing an RNA polymerase promoter and an insert of interest is preferably first linearized downstream of the insert, by e.g., restriction digest with an appropriate restriction enzyme. The linearized DNA is then incubated for about 1 hour at 37° or 40° C. (depending on the RNA polymerase) in the presence of ribonucleotides, an RNAase inhibitor, an RNA polymerase recognizing the promoter that is operably linked upstream of the insert to be transcribed, and an appropriate buffer containing TrisHCl, MgCl₂, spermidine and NaCl. Following the transcription reaction, RNase free DNAse can be added to remove the DNA template and the RNA can be purified by, e.g., a phenol-chloroform extraction. Usually about 5-10 μg of RNA can be obtained per microgram of template DNA. Further details regarding this protocol are set forth, e.g., in Molecular Cloning A Laboratory Manual, 2nd Ed., ed. by Sambrook, Fritsch and Maniatis (Cold Spring Harbor Laboratory Press: 1989).

In another embodiment, the RNA is “capped,” i.e., that the 5′ nucleotide at the 5′ end of the RNA has a 5′-5′ linkage with a 7-methylguanylate (“7-methyl G”) residue. The presence of a 7-methyl G on an RNA molecule in a 5′-5′ linkage is referred to as a “cap.” Thus, in one embodiment, the RNA is contacted with methyl-7 (5′) PPP (5′) guanylate (available, e.g., from Boehringer Mannheim Biochemicals) in the presence of an in vitro transcription reaction mixture, to obtain capped RNA. In the case of in vitro transcribed RNA, capping is preferably carried out during in vitro transcription.

In order to prevent RNA from being degraded by nucleases, e.g., by RNases, that may be present, the extraction of RNA, and reactions involving RNA are performed in “RNase free conditions.” Various methods known in the art can be used to maintain RNase free conditions. For example, during RNA extraction, potent denaturing agents, such as guanidium hydrochloride and guanidium thiocyanate can be used to denature and thereby inactivate nucleases. Reducing agents, e.g., β-mercaptoethanol, can also be used to inactivate ribonucleases. This combination of agents is particularly useful when isolating RNA from tissues rich in ribonucleases, e.g., pancreas (Chirgwin et al., (1979) Biochemistry 18:5294).

Other reagents that can be added to a solution containing RNA to prevent degradation of the RNA include RNase inhibitors, also referred to herein as “protein inhibitor of RNases,” e.g., RNasin (.which can be obtained, from Promega Corp. (Madison, Wis.) (e.g., Cat #N2514). Protein inhibitors of RNases are preferably not included during extraction of RNA using potent denaturing agents (since these will also denature the protein inhibitor of RNases). However, it is preferable to include such protein inhibitors of RNases during RNA extraction using more gentle methods of cell lysis and RNase inhibitors are preferably present at all stages during the subsequent purification of RNA.

Yet another reagent that can be added to a solution containing RNA to prevent degradation of the RNA include vanadyl-ribonucleoside complexes. The complexes formed between the oxovanadium IV ion and any of the four ribonucleosides are transition-state analogs that bind to many RNases and inhibit their activity almost completely. The four vanadyl-ribonucleoside complexes are preferably added to intact cells and preferably used at a concentration of 10 mM during all stages or RNA extraction and purification. Yet in another embodiment, macaloid is used to absorb RNases.

RNA can also be extracted from cells or tissues according to methods known in the art. In a preferred embodiment, RNA can be extracted from monolayers of mammalian cells grown in tissue culture, cells in suspension or from mammalian tissue that can readily be dispersed in single cells. RNA can be extracted from such sources by, e.g., treating the cells with proteinase K in the presence of SDS. In another embodiment, RNA is extracted by organic solvents. In yet another embodiment, RNA is extracted by differential precipitation to separate high molecular weight RNA from other nucleic acids. RNA can also be extracted from a specific cellular compartment, e.g., nucleus or the cytoplasm. In such methods, the nucleus is either isolated for purification of RNA therefrom, or the nucleus is discarded for purification of cytoplasmic RNA. Further details regarding these and other RNA extraction protocols are set forth, e.g., in Molecular Cloning A Laboratory Manual, 2nd Ed., ed. by Sambrook, Fritsch and Maniatis (Cold Spring Harbor Laboratory Press: 1989).

For instance, RNA can be extracted by a method using guanidium thiocyanate and purification of the RNA on a cesium chloride gradient. Accordingly, tissue or cells are lysed in the presence of guanidium thiocyanate and the cell lysate is loaded on a cushion of cesium chloride (CsCl) and centrifuged at high speed, such that the RNA is recovered in the pellet and the DNA is left in the supernatant after the centrifugation. The RNA can then be recovered by ethanol precipitation. This method is set forth in detail, e.g., in Molecular Cloning A Laboratory Manual, 2nd Ed., ed. by Sambrook, Fritsch and Maniatis (Cold Spring Harbor Laboratory Press: 1989).

### Example 4

### Exogenous Gene Delivery of Ribozymes

Pre-formed ribozymes can be delivered exogenously using, for example, lipids, synthetic polymers, electroporation or microinjection. These ribozymes may require the introduction of modified nucleotides, to protect them from nucleases present in cells. There are three major target sites for modification present in RNAs: the base (A, U, C or G), the sugar, and the internucleotide phosphodiester linkage, where individual bases are linked to one another. Modified RNAs can be generated using chemical or enzymatic techniques.

For instance, RNAs transcribed by polII contain a 5′-cap which protects them against 5′-exoribonucleases (Furuichi et al., 1977). Thus if pre-formed ribozymes are generated with the 5′-cap they are partially protected from nuclease degradation. In addition, the 5′-end of a ribozyme (the first base) can comprise of a modified oligonucleotide containing modifications such as guanosine, guanosine 5′-monophosphate (GNT), guanosine 5′-O-(1-thiomonophosphate), ApG, CpG, AmpG, and others (reviewed in Gaur and Krupp, 1997). Oligonucleotides (either DNA or RNA) containing phosphorothioate linkages (on the sugar) can be synthesized by either chemical or enzymatic means (Eckstein, 1985) and are protected from degradation by endonucleases. In addition the 2′-hydroxyl groups (on the base) in RNAs can be exchanged for 2′ fluoro-, 2′-amino-, 2′-O-methyl-, 2′-O-allyl or 2′deoxy-nucleotides. These RNAs can be generated using chemical or enzymatic approaches. In addition, 2′-modified bases can be introduced at specific sites in chemically synthesized ribozymes, although only short RNAs can be synthesized chemically.

One method of RNA modification has been evaluated for RzPolCol1A1, targeting a polymorphic site (C3210T) in the COL1A1 transcript. RzPolCol1A1 cleaves the T-allele at 3210 in vitro, while leaving COL1A1 transcript with a C at position 3210 (C-allele) of the COL1A1 transcript intact (Millington-Ward et al., 1997; Millington-Ward et al., 1999). In preparation for exogenous delivery of RzPolCol1A1 to cells, the ribozyme was enzymatically generated, by in vitro transcription, with the incorporation of 2′-aminonucleotides; at both uracil and cytocine bases. The modified ribozyme was tested both for stability in serum and cleavage ability at position 3210 of the COL1A1 T-allele transcript. The incorporation of 2′-fluoro- and 2′-aminonucleotides into hammerhead ribozymes has previously been found to be a method of stabilizing the molecule (Pieken et al., 1991a). However, the catalytic efficiency of the ribozyme may be reduced by this modification, depending on how many bases are modified and where these are located (Pieken et al., 1991b; Heidenreich and Eckstein, 1992). The mechanism of inhibition of cleavage efficiency was explored in a study evaluating the effect(s) of each base substitution (Leirdal and Sioud, 1998). The study showed that efficient cleavage activity could be achieved for 2′-amino modified ribozymes when their sequences contained only a few pyrimidines in helix I (the antisense arm of the ribozyme that binds 3′ to the NUX site). Ribozymes with no pyrimidines in helix I cleave target RNAs with almost the same efficacy as unmodified ribozymes.

### Example 5

### Endogenous Gene Delivery of Ribozymes

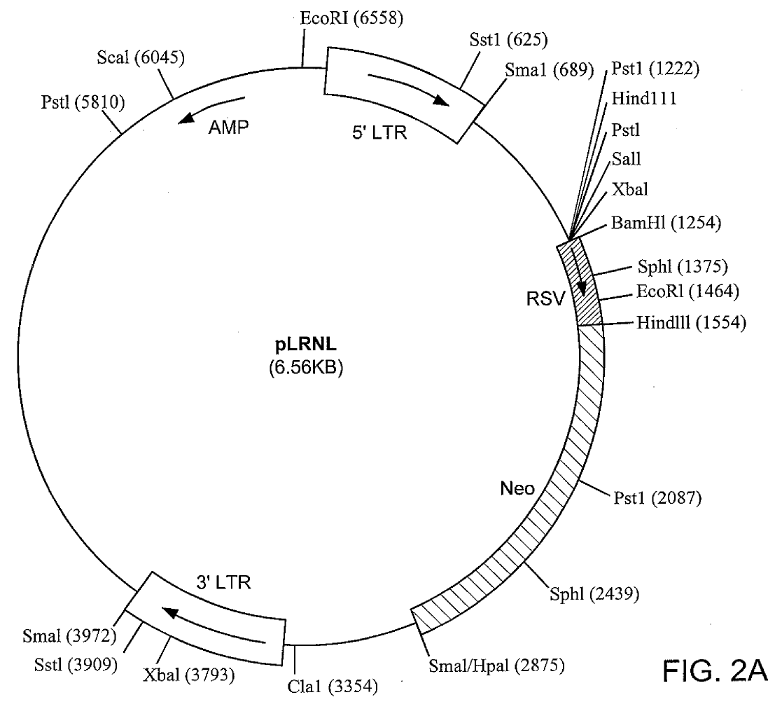

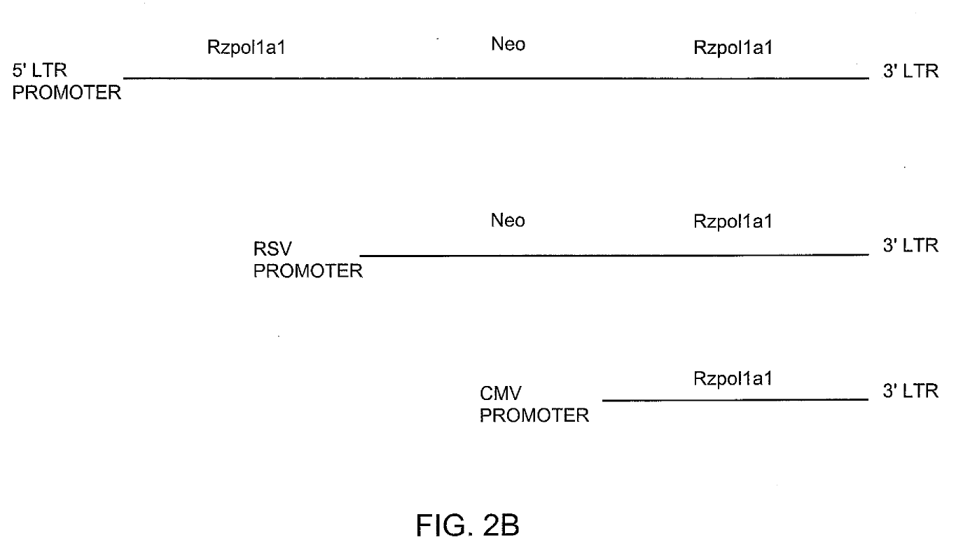

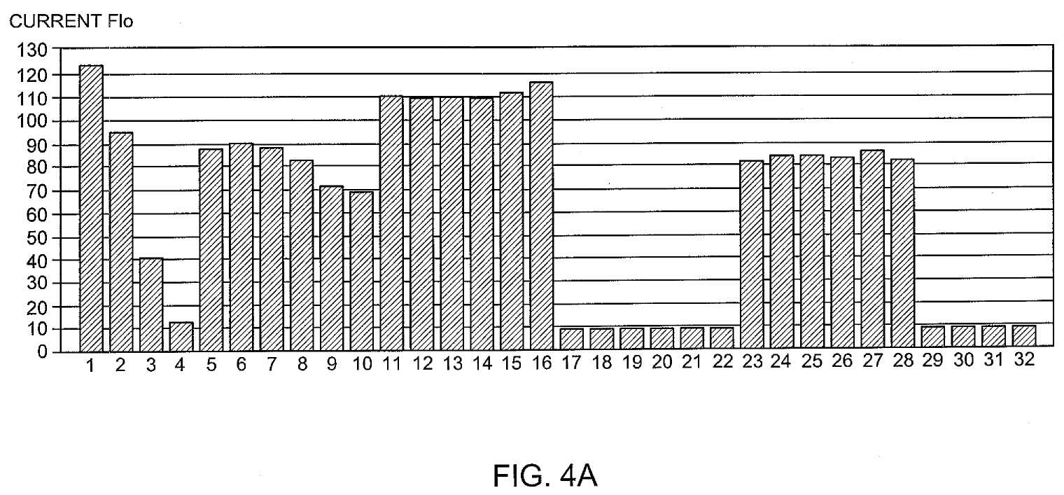

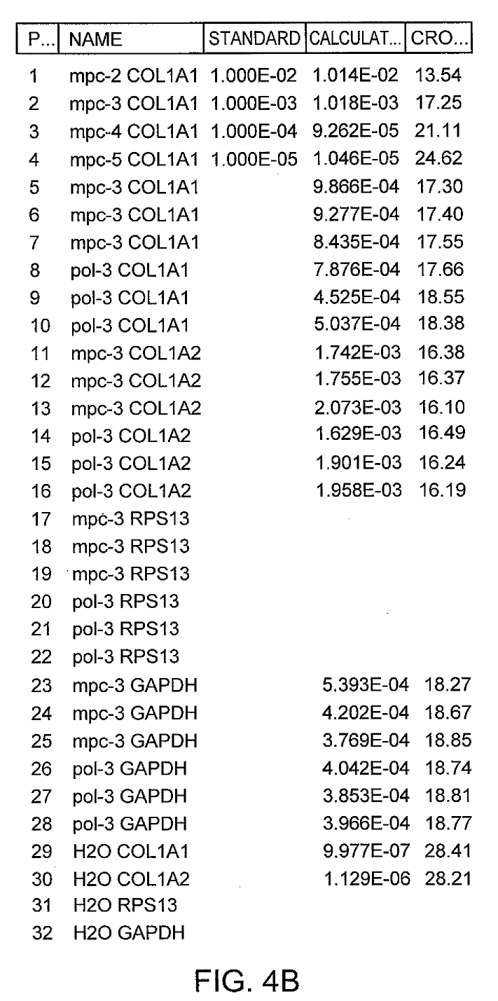

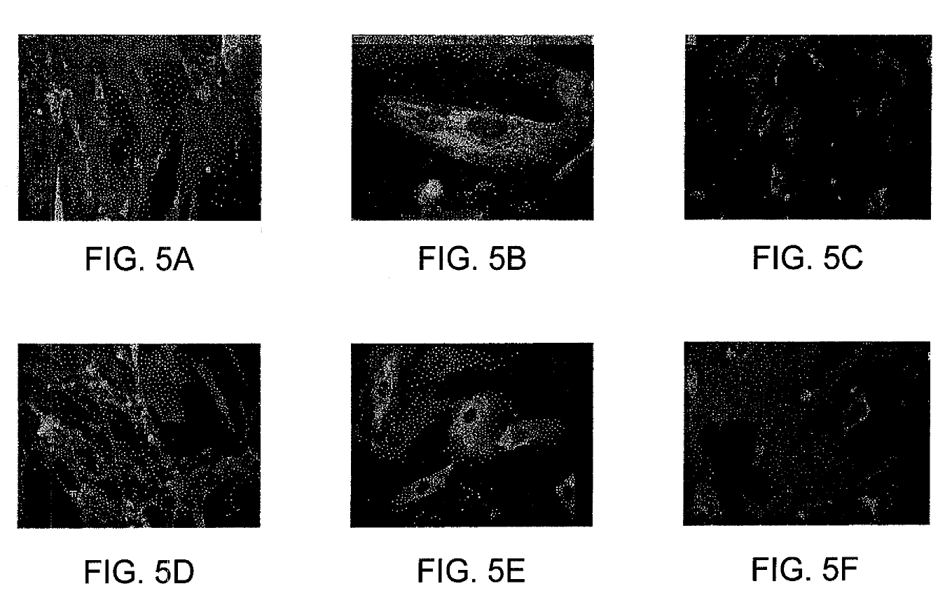

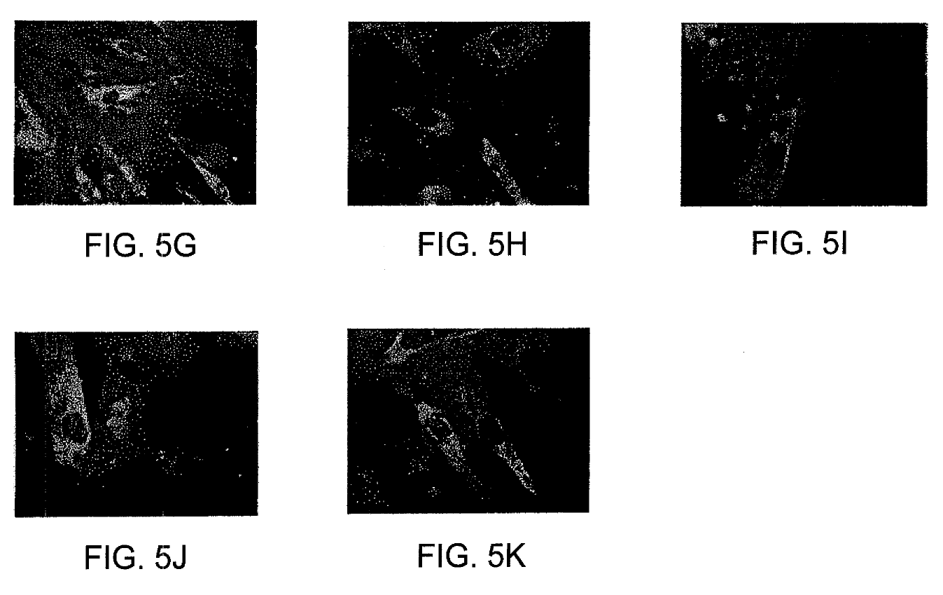

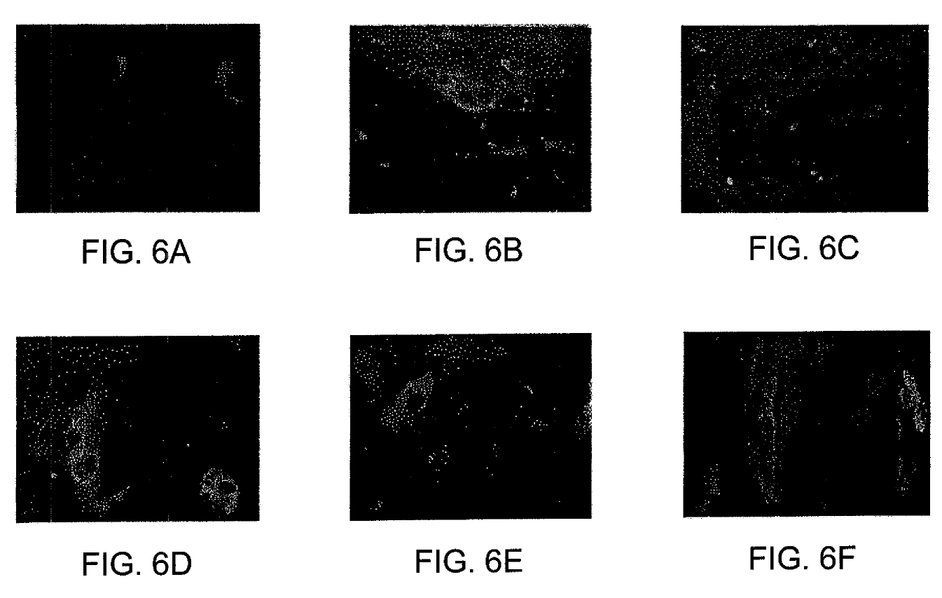

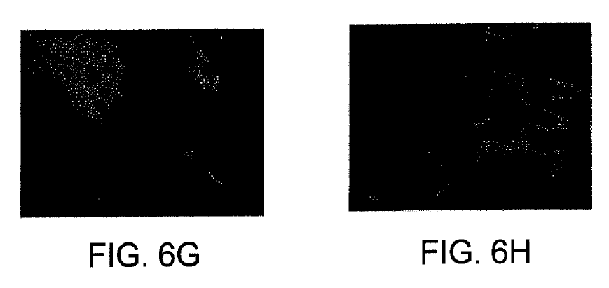

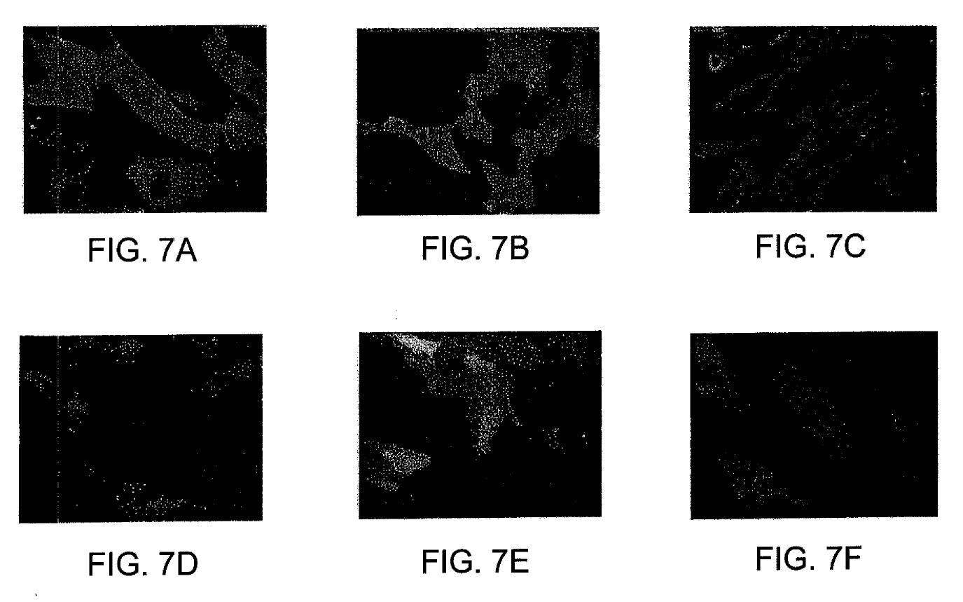

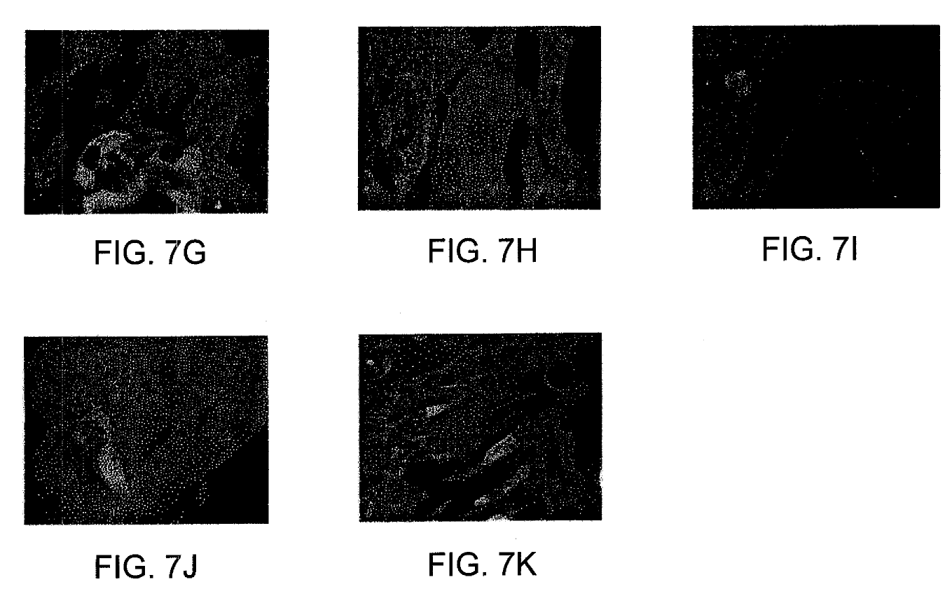

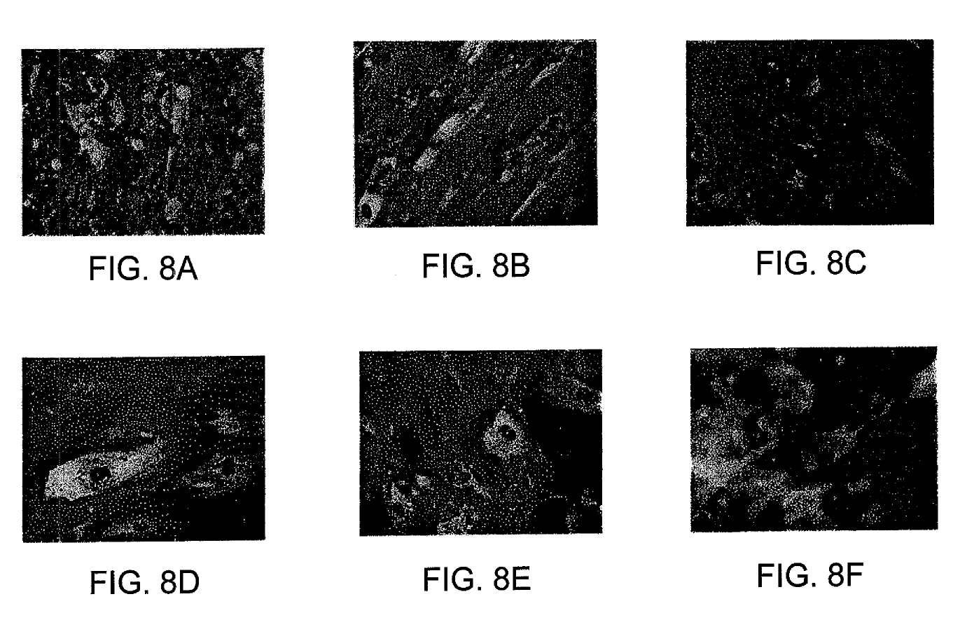

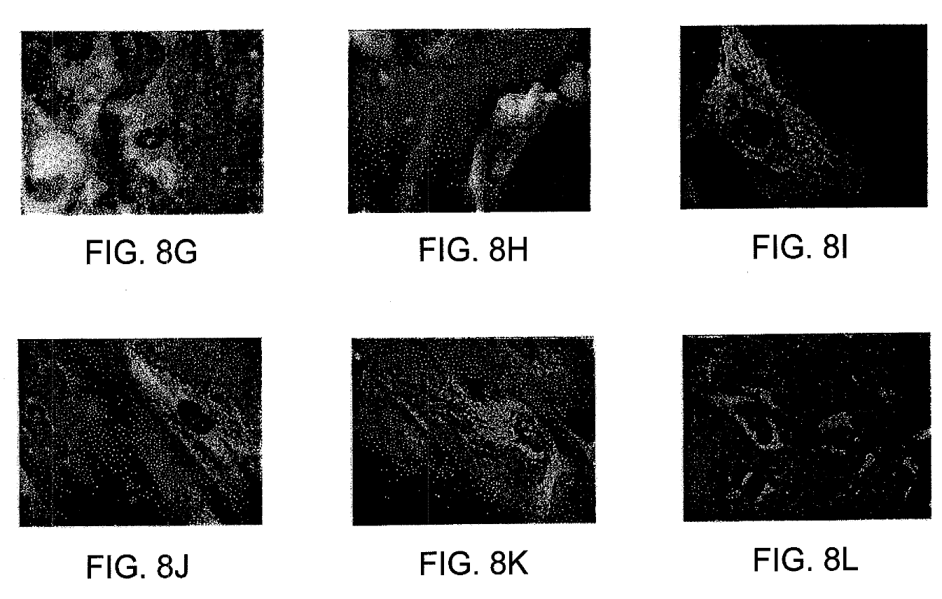

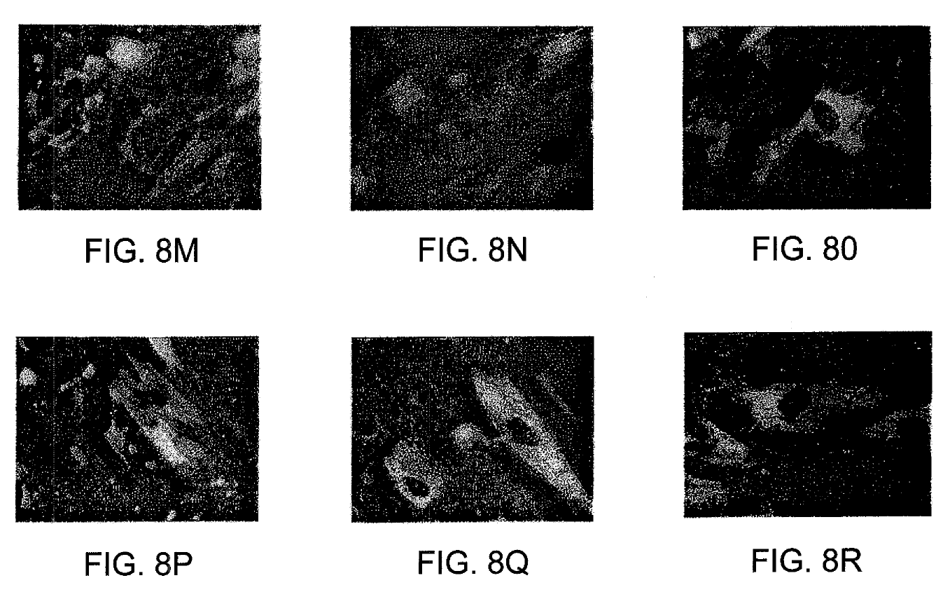

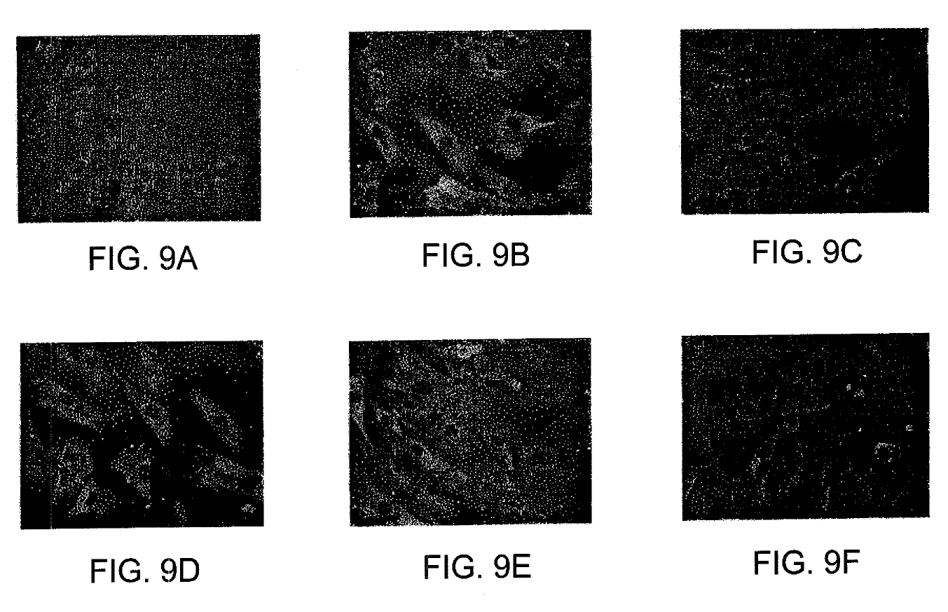

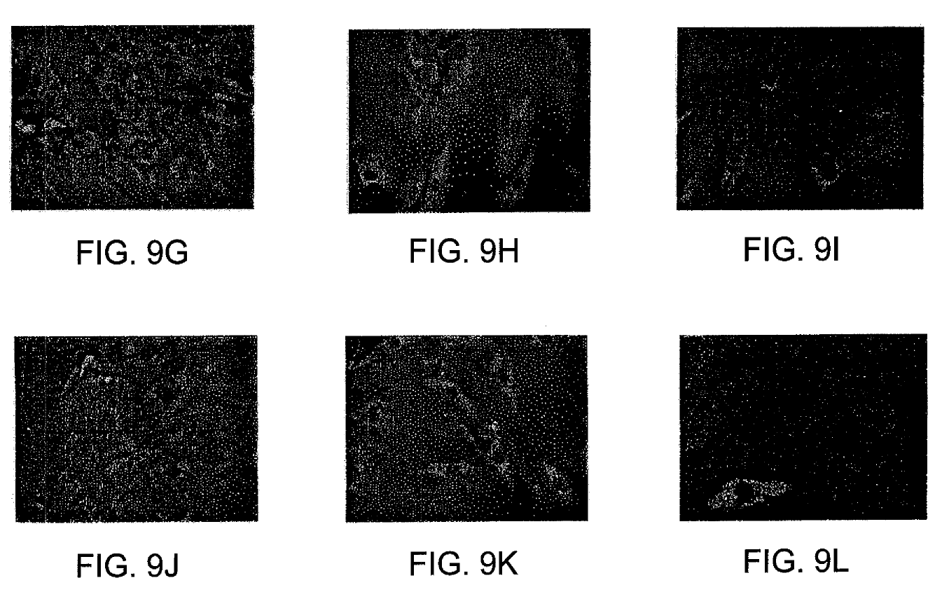

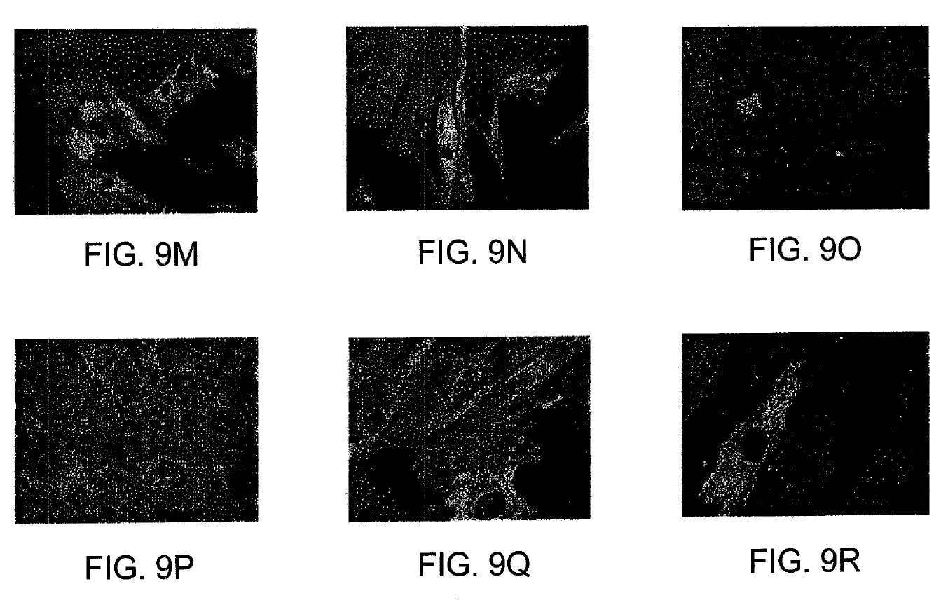

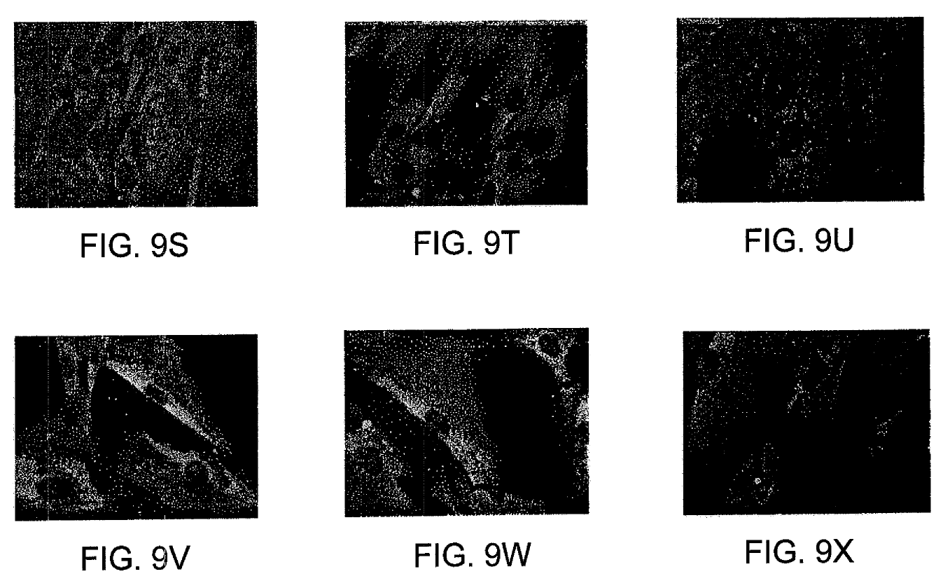

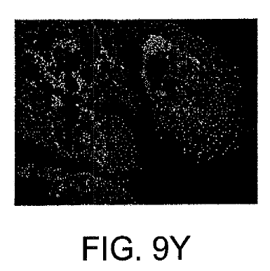

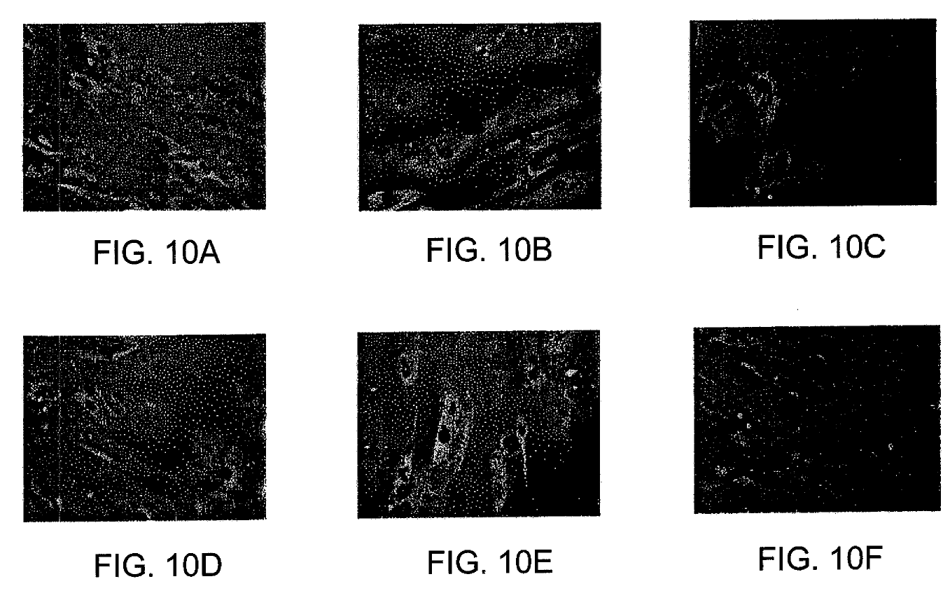

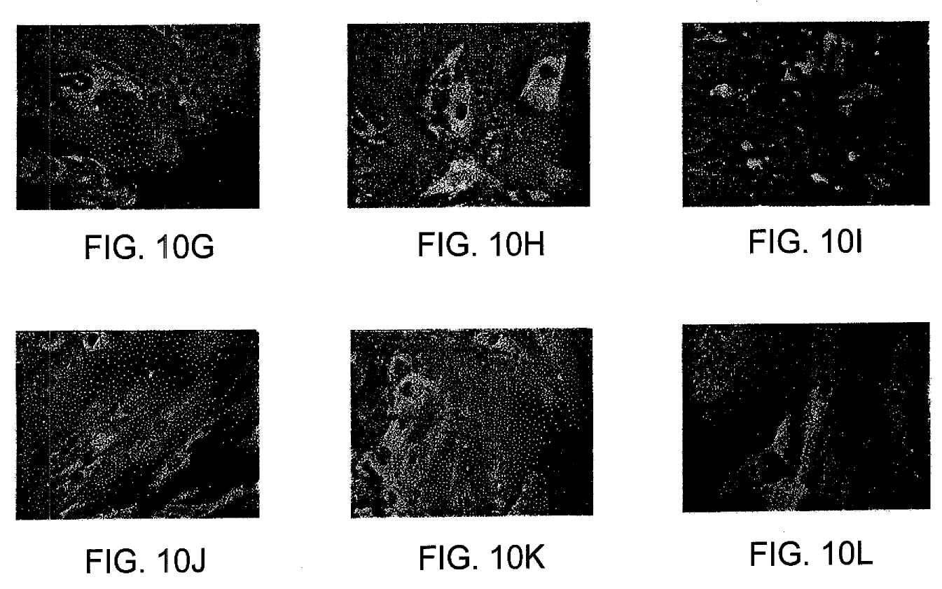

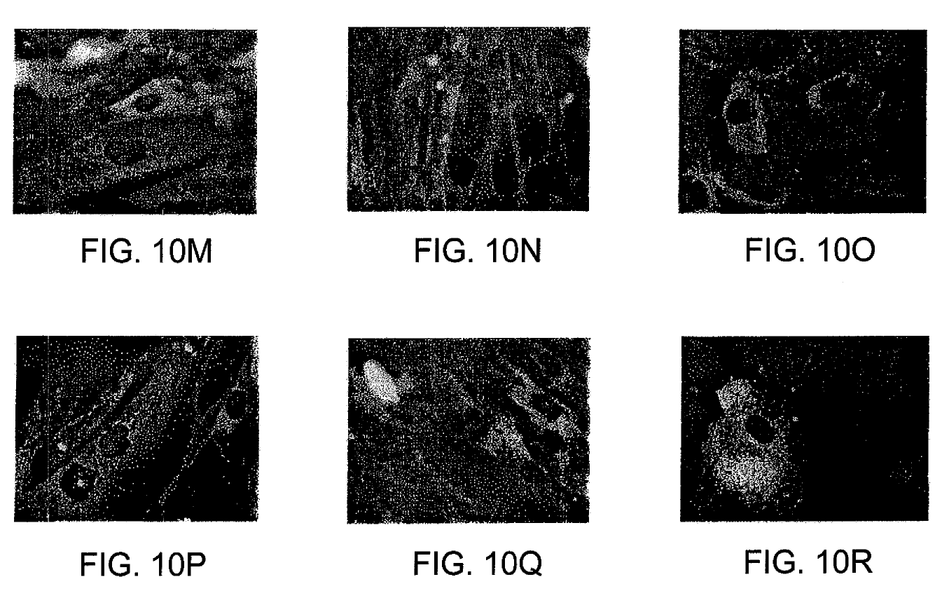

In addition to delivering preformed ribozymes (or indeed any genetic therapeutic), to a target tissue exogenously, endogenous gene delivery to cells is performed. Typically endogenous gene delivery involves use of transcriptional units such as plasmids or viruses. For this reason a retroviral vector with RzPolCol1A1 was generated. RzPolCol1A1 was cloned into two sites of plasmid pLRNL (Yee et al., 1994), a retroviral plasmid. One copy of RzPolCol1A1 was cloned in the Sal I and BamH I sites of pLRNL. In addition, a second RzPolCol1A1 driven by a CMV promoter was cloned into the Cla I site of pLRNL (FIG. 2A). Thus, any retrovirus generated with pLRNL will therefore give rise to three different RNA molecules containing either one or two copies of RzPolCol1A1 (FIG. 2B). The cloning of RzPolCol1A1 has been verified by automated sequencing. Resulting retrovirus has been used to stably transfect human mesenchymal progenitor stem cells (MPCs) carrying two T-alleles of the COL1A1 gene i.e. homozygous 3210T cells. Infections were carried out at MOI=5. Stable cells expressing viral RNA were selected after 48 hours with G418 and grown in bulk. Cellular RNA was isolated from MPCs stably expressing the retrovirus using TRIzol (GibcoBRL) and the manufacturer's protocols. RNAs were tested for ribozyme activity using real time RT PCR and the following two primer pairs given in a 5′ to 3′ orientation: NeoF GATGCCTGCTTGCCGAATAT NeoR GCTCTTCAGCAATATCACGG and pLRNLF AAGACAGGATATCAGTGGTC and pLRNLR CTATGGCTCGTACTCTATAG (Table 4). MPC cell lines were evaluated for COL1A1 down-regulation at both at the RNA and protein level using real time RT PCR and fluorescent anti-collagen antibodies respectively. RNA levels were shown to be down-regulated by 38.8% in 7 different experiments, each carried out in triplicate (i.e., 21 samples in all). In 4 of the 7 experiments total RNA levels were normalized to the housekeeping gene GAPDH using the following primer pair in a 5′ to a 3′ orientation: GAPDHF CAGCCTCAAGATCATCAGCA and GAPDHR CATGAGTCCTTCCACGATAC. In 3 of the 7 experiments total RNA levels were normalized to COL1A2 RNA levels; the natural associate of COL1A1 (FIG. 4). Primers used given in a 5′ to 3′ orientation were the following: COL1A2F CAAGGATGCACTATGGATGC and COL1A2R GGAGCTCCTATACCAGTTCT. This is the first ribozyme to have been shown to be active in MPCs, though ribozymes have been shown to be active in other cell types. Moreover this is the first ribozyme to have been shown to target a polymorphic site. To corroborate this result at the protein level and to confirm that down-regulation at the RNA level is indeed reflected at the protein level, stable MPC lines were treated with a FITC-labeled anti-type I collagen antibody. Briefly, 20,000 cells were grown on a coverslip for either 4 or 7 days, rinsed once in PBS, drained and fixed for 2 minutes in 50% acetone and 50% methanol. Fixed cells were rinsed with PBS+3% BSA and treated for 30 minutes with a 1/200 dilution of anti human type I collagen raised in mouse (Biodirect) at room temperature in a humid chamber. Cells were then washed twice over 5 minutes with PBS+3% BSA. Subsequently cells were treated at room temperature for 20 minutes with a 1/64 dilution of goat anti mouse IgG-FITC (Sigma) in a humid chamber. Cells were then washed twice in PBS over 5 minutes. Cells were mounted on a glass slide with Moial and analyzed with a fluorescent microscope using 100× and 200× magnifications (FIGS. 5-10). Cells expressing RzPolCol1a1 (Pol 1,2 and Pol 3 cells) were compared to control MPCs treated in the same manner after 4 and 7 days growth. In both cases a significant down-regulation of type I collagen was visible at the protein level, indicating that RzPolCol1a1 is highly active in human MPCs and in addition that a down-regulation at the RNA level is indeed mirrored at the protein level (FIGS. 5-10). Stable lines were grown in osteogenic medium (0.1 μM dexamethasone, 10 mM beta-glycerophosphate and 50 μM ascorbate-2-phosphate) for 7 days and were shown to have maintained their ability to differentiate into osteoblasts. Clearly a ribozyme delivered to MPCs by a retroviral vector targeting a polymorphic site has achieved significant levels of down-regulation at the RNA and protein level. Thus, suppression of human COL1A1 in the target human stem cells (MPCs) that give rise to bone tissue has been demonstrated. Cell lines with high levels of ribozyme activity and COL1A1 suppression may now be transfected with a second retrovirus carrying a replacement COL1A1 gene, with a C at position 3210 of the gene. Replacement COL1A1 transcripts are protected from suppression by RzPolCol1A1. Replacement COL1A1 genes could be driven by strong cell specific promoters such as the COL1A1 or COL1A2 promoters and would most likely contain appropriate intronic sequences to ensure high levels of expression. In addition, replacement gene could include sufficient quantities of 3′ downstream sequence to ensure replacement transcripts were sufficiently stable. A 2.3 kb portion of the COL1A1 promoter, the 1.6 kb first intron of COL1A1 and a 2 kb portion of 3′ downstream sequence of the COL1A1 are known to drive normal levels of COL1A1 expression in transgenic mice (Sokolov et al. 1993) and may thus be used in replacement constructs. Replacement COL1A1 constructs would not only carry a C at the polymorphic site at position 3210, but may also include additional change at positions adjacent to the cleavage site to minimize antisense effects of the ribozyme. Since the polymorphism is situated in the 3′UTR of COL1A1, small sequence alterations should not interfere with normal protein expression. Double stable MPC lines expressing both RzPolCol1A1 and the replacement construct are also generated to test the allele suppression and replacement methods of the invention. Cells, which show high levels of replacement collagen 1A1 expression, may then be injected into a COL1A1-linked OI patient's bone marrow or may be administered systemically, giving rise to generations of osteoblasts and chondrocytes expressing wild-type COL1 A1. Notably, the systemic administration of donor bone marrow cells to leukemia patients has been carried out routinely for many years. One elegant feature of the suppression and replacement method utilizing ex vivo gene delivery is that a patient's own mesenchymal progenitor stem cells may be used as gene delivery vehicles, most probably making the lifelong administration of immunosuppressors to the OI patients unnecessary.

Table 4

| | | | SEQ |
|---|---|---|---|
| | | | ID |
| Primer name | RNA Amplified | Primer Sequence | NO |
| COL1A1F | Collagen 1A1 | CAGGAATTCGGCTTCGA | 25 |
| COL1A1R | Collagen 1A1 | GGTTCAGTTTGGGTTGCTTG | 26 |
| COL1A2F | Collagen 1A2 | CAAGGATGCACTATGGATGC | 27 |
| COL1A2R | Collagen 1A2 | GGAGCTCCTATACCAGTTCT | 28 |
| GAPDHF | GAPDH | CAGCCTCAAGATCATCAGCA | 29 |
| GAPDHR | GAPDH | CATGAGTCCTTCCACGATAC | 30 |
| NeoF | Neo | GATGCCTGCTTGCCGAATAT | 31 |
| NeoR | Neo | GCTCTTCAGCAATATCACGG | 32 |
| pLRNLF | pLRNL | AAGACAGGATATCAGTGGTC | 33 |
| pLRNLR | pLNRL | CTATGGCTCGTACTCTATAG | 34 |

### Example 6

### Polymorphism and Genomics Driven Drug Design

Only recently is polymorphism being recognized for its enormous potential in genomics-driven drug design. This is demonstrated by aims of the Single Nucleotide Polymorphism (SNP) Consortium Ltd., as part of the human genome project, to characterize 150,000 SNPs distributed evenly throughout the human genome by the middle of 2001 (http://www.ncbi.nlm.nih.gov/SNP/). There are estimated to be 3 million SNPs in the human genome (http://www.ornl.gov/hgmis/faq/snps.html). In addition, many commercial companies, such as ORCHID Biosciences, Inc., have been set up for the sole purpose of characterizing SNPs and identifying proprietary medical applications of SNPs (http://www.noonanrusso.com/clients/bulbs/orchid.html). However, to date polymorphism has not been utilized as a tool for down-regulating in cells transcripts with dominant-negative mutations or mutations which predispose to pathology, whereby mutant alleles are specifically suppressed at SNPs and wild-type non-suppressible alleles of alternative polymorphic genotype are concurrently administered to cells of subjects. The strategy of allele suppression and replacement is demonstrated herein for the autosomal dominant brittle bone disorder OI. In addition, suppression of the rhodopsin transcript has been demonstrated in vitro. The allele suppression methods of the instant invention can be applied to cells with many other genes giving rise to or predisposing to dominant negative phenotype(s). Other common polymorphic genes which, when mutated, give rise to dominant negative ‘diseases or predispose to disease pathologies include amongst others P53, APC, FBN1 are presented in (Tables 5-11). For example, a significant number of tumors have dominant gain of function mutations in P53 (http://perso.curie.fr/Thierry.Souissi/p53_mutation.html). In addition, well over 100 P53 mutations have been characterized (http://www.lf2.cuni.cz/projects/dbu99_{—}08.txt). A portion of these are gain of function mutations and are therefore appropriate for the allele suppression and replacement methods of the invention. Table 5 shows polymorphic sites in a number of genes that create NUX sites and would therefore be suitable for allele specific suppression using hammerhead ribozymes.

Apolipoprotein E (ApoE) fulfills fundamental functions in lipid transport and neural tissue repair after injury. Its three most common isoforms (E2, E3 and E4) are critical determinants of diverse human diseases, including major cardiovascular and neurodegenerative disorders. ApoE4 is associated with an increased risk for Alzheimer's disease and poor clinical outcome after head injury or stroke. Recently, the effects of ApoE4 have been shown to be dominant negative in transgenic mouse models expressing the E3, E4 or both alleles (Buttini et al. 2000). In contrast, ApoE2 has been found to be associated with some vascular abnormalities, specifically, a “double-barrel” appearance and fibrinoid necrosis (McCarron et al. 2000). In addition to the three common ApoE isoforms, a number of other polymorphisms have been characterized in the gene (Table 11). Thus, the allele suppression strategy could be applied to either ApoE2 or ApoE4 related disease, using any of the polymorphisms described or indeed polymorphisms which have not as yet been characterized and a suppression agent. For example, the 783 mutation (Table 11) involves a polymorphism (c-t), which creates a NUX ribozyme cleavage site. The polymorphism is situated in a predicted open loop structure according to Plotfold analysis. Notably, the replacement gene could be of the ApoE3 isoform, an isoform which has not been associated with either neurological or vascular abnormalities. Thus, if a patient with ApoE4 linked Alzheimer's disease is heterozygous with one copy of the ApoE2 and one copy of the ApoE4 gene the replacement ApoE3 construct may treat the Alzheimer's disease and in addition protect the patient from vascular disease.

Many genes harboring dominant negative mutations or a mutation predisposing to disease pathologies demonstrate high levels of intragenic polymorphism (in some cases at NUX ribozyme target sites) and would therefore lend themselves to the selective allele suppression and replacement strategy (Table 5). The instant invention circumvents the problem of genetic heterogeneity inherent in such genes and provides methods and strategies for suppressing and/or replacing these genes. Digenic or polygenic disorders may also be treated by the methods and compositions of the invention. For example, digenic RP is caused by mutations at the unlinked peripherin/RDS and ROM1 loci (Clarke et al. 1998); Kajiwara et al. 1994). In addition, digenic autosomal dominant deafness is caused by disease haplotypes at both the DFNA2 and DFNA12 loci (Borg et al. 2000).

TABLE 5
Polymorphisms in heterogeneous dominant negative disease genes and intragenic
SNPs that create NUX ribozyme cleavage sites

| Disease | Gene | Polymorphic Site | Reference |
|---|---|---|---|
| OI | COL1A1 | C3210T | Westerhausen et al. Matrix |
| | | | 11: 375-379 (1991) |
| OI | COL1A2 | GTA868GTG | Filie et al. Hum Mutat 2: 380-388 |
| | | | (1993) |
| OI | COL1A2 | T385C | Strobel et al. Matrix 12: 87-91 ( |
| OI | COL1A2 | T225C | Zhuang et al. Hum Mutat 7: 89-99 |
| | | | (1996) |
| RP | Peripherin/ | Catcc-caccc 2046 in | http://www.irpa.org/sci-news/rdsmut.htm |
| | RDS | 3′UTR | |
| RP | Rhodopsin | C1090T | Saga et al. Ophthalmic Genet. |
| | | | 15: 61-67 (1994) |
| RP | Peripherin/ | G611T | Shastry et al. Biochem. |
| | RDS | | Biophys. Res. Com. 231: 103 |
| | | | (1997) |
| RP | Peripherin/ | A566G | Shastry et al. Biochem. |
| | RDS | | Biophys. Res. Com. 231: 103 |
| | | | (1997) |
| Cancer | P53 | GAC-GAT (codon | NM 000546; Ahuja et al. |
| | | 21/base 314) | Oncogene 5: 1409-1410 (1990) |
| Cancer | P53 | CCG-TCG (codon | NM 000546; Felley-Bosco et |
| | | 47/base 390) | al. Am J Hum Genet 53: 752-759 |
| | | | (1993) |
| | | | 4.7% in african americans |
| Cancer | P53 | CCG-GTC (codon | NM 000546; Murakami et al. |
| | | 189/base 817) | Cancer Res 51: 5520-5525 |
| | | | (1991) |
| Cancer | APC | GGA-GTA (codon | NM 000038 Powel et al. |
| | | 84/base 289) | Nature 359: 235-357 (1992) |
| Cancer | APC | TAT-TAC (codon | NM 000038 Miyoshi et al. |
| | | 486/base 1497 | Proc Natl Acad Sci (USA) |
| | | | 89: 4452-4456 (1992) |
| Alzheimer's | ApoE | GTG-GTA at base 369 | Accession No. K00396 |
| Alzheimer's | ApoE | ATG-ATC at base 568 | Accession No. K00396 |
| Alzheimer's | ApoE | GTC-GCC at base 783 | Accession No. K00396 |
| Deafness and | COL11A2 | GTC-GAC at base 7167 | Accession No. U32169 |
| other disorders | | | |
| Epidermolytic | Keratin 10 | GTT-GCT at base 3513 | Accession No. X14487 |
| hyperkeratosis | | | |
| Epidermolysis | Keratin 5 | TCT-TTT at base 42 | Accession No. U05845 |
| Bullosa Simplex | | | |
| Dystrophic | COL7A1 | GTC-GTG at base 9164 | Accession No. L02870 |
| Epidermolysis | | | |
| Bullosa | | | |
| EDS IV | COL3A1 | GGC2346GGT (codon | Tromp et al. Nucl Acids Res |
| | | 581) | 19: 681 (1991) |

TABLE 6
Polymorphisms in human Peripherin (RDS)
(http://www.irpa.org/sci-news/rdsmut.htm)

| Variation | Sequence | Nucleotide | Exon |
|---|---|---|---|
| (TA)n | | | 5′UTR |
| Tyr83Tyr | TAC-TAT | 0489 | 1 |
| Tyr101Tyr | TAC-TAT | 0543 | 1 |
| Val106Val | GTC-GTT | 0558 | 1 |
| Tyr236Tyr | TAC-TAT | 0948 | 2 |
| Glu304Gln | GAG-CAG | 1150 | 3 |
| Lys310Arg | AAG-AGG | 1169 | 3 |
| Pro313Leu | CCG-CTG | 1178 | 3 |
| Gly338Asp | GGC-Gac | 1253 | 3 |
| 1426G-A | G/A | 1426 | 3′UTR |
| 18271-g | Tacac-tacgc | 1827 | 3′UTR |
| 2046t-c | Catcc-caccc | 2046 | 3′UTR |
| 2131del4bp | Aatcaga-a_a | 2131 | 3′UTR |
| 2182a-c | Gccaa-gccca | 2182 | 3′UTR |
| 2285-1-c | Caaga-ccaga | 2285 | 3′UTR |
| 2641t-c | Ctttg-ctctg | 2641 | 3′UTR |
| 2659t-c | Tagtg-cagtg | 2659 | 3′UTR |

TABLE 7
Polymorphisms in Human MSH gene
(http://www.nfdht.nl/database/msh2-poly.htm)

| Exon | Codon | Nucleotide | Codon |
|---|---|---|---|
| Intron 1 | +9 of Ex 1 | c-g at 211 + 9 | |
| 2 | 113 | AAG-AAA | Lys-Lys |
| 2 | 110 | A-G at 329 | Lys-Arg |
| 2 | 96 | G-A at 287 | Arg-His |
| 2 | 113 | G-A at 339 | Lys-Lys |
| Intron 2 | 5′ poly-T region | Variable T deletion | |
| 3 | 133 | C-T at 399 | Asp-Asp |
| 3 | 153 | C-T at 459 | Ser-Ser |
| Intron 5 | Poly-A region | 10 bp del of A | |
| 6 | 322 | GGC-GAC at 965 | Gly-Asp |
| 6 | 328 | GCC-GCT | Ala-Ala |
| Intron 6 | −10 of 5′Ex 7 | t-c at 1077 − 10 | |
| Intron 7 | +49 of 3′Ex 7 | C-A at 1276 + 49 | |
| Intron 9 | −10 of 5′Ex 10 | g-t at 1511 − 10 | |
| Intron 9 | −9 of 5′Ex 10 | t-a at 1511 − 9 | |
| Intron 10 | +12 of 3′Ex 10 | g-a at 1661 + 12 | |
| 10 | 521 | T-A at 1563 | Tyr-Tyr |
| 11 | 556 | T-C at 1666 | Leu-Leu |
| 11 | 579 | A-G at 1737 | Lys-Lys |
| 11 | 585 | TCT-TCC at 1755 | Ser-Ser |
| 12 | 596 | A-G at 1787 | aSN-sER |
| Intron 12 | −6 of 5′Ex 13 | t-c at 2006 − 6 | |
| 13 | 713 | G-C at 2139 | Gly-Gly |
| 13 | 718 | A-G at 2154 | Gln-Gln |

TABLE 8
Polymorphisms in Human Collagen COL3A1

| Polymorphism | Reference |
|---|---|
| Pro501Thr | Tromp et al. J Clin Invest 91: 2539-2545 |
| CCT-ACT nt2104 | (1993) |
| Ala531Thr | Zafarullah et al. Nucl Acids Res 18: 6180 |
| GCT-ACT nt2194 | (1990) |
| Gly581Gly | Tromp et al. Nucl Acids Res 19: 681 (1991) |
| GGC-GGT nt2346 | |

TABLE 9
Polymorphisms in Human Collagen COL1A1
http://www.le.ac.uk/ge/collagen/col1a1.html#Table_4

| Polymorphism | Base | Reference |
|---|---|---|
| Arg59Arg CGG-CGT | N-propeptide nt296 | Mackay et al. Hum Mol Genet 2: 1155-1160 |
| | | (1993) |
| Pro228Pro CGG-CGT | 1667 | Marini et al. J Biol Chem 268: 2667-2673 |
| | | (1993) |
| Arg386His CGC-CAC | 1810 | Pruchno et al. Hum Genet 87: 33-40 (1991) |
| Ala410Ala GCT-GCC | 1883 | Mackay et al. Hum Mol Genet 2: 1155-1160 |
| | | (1993) |
| Gly-517Gly GGA- | 2204 | Nicholls et al. J Med Genet 28: 757-764 (1991) |
| GGT | | |
| Pro645Ala CCT-GCT | 2586 | Mackay et al. Hum Mol Genet 2: 1155-1160 |
| | | (1993) |
| Ala897Thr GCC-ACC | 3342 | Sokolov et al. Nucl Acids Res 19: 4302 (1991) |
| Pro899Pro CCT-CCC | 3350 | Lamande et al. J Biol Chem 264: 15809-15812 |
| | | (1989) |
| Pro902Pro CCT-CCC | 3359 | Lamande et al. J Biol Chem 264: 15809-15812 |
| | | (1989) |
| Val903Val GTC-GTT | 3362 | Bateman et al. Am J Med Genet 45: 233-240 |
| | | (1993) |
| Asp975Asp GAT- | 2578 | Zhuang et al. Hum Mutat 7: 89-99 (1996) |
| GAC | | |
| Ser1215Ser TCC-TCT | 4298 | Zhuang et al. Hum Mutat 7: 89-99 (1996) |
| Ser1256Thr TCC- | C-propeptide 4419 | Makela et al. Nucl Acids Res 16: 349 (1988) |
| ACC | | |
| TCA-CCA | 3′UTR 4602 | Zhuang et al. Hum Mutat 7: 89-99 (1996) |
| AGCA insertion | 3′UTR insertion following | Nuytinck et al. Matrix Biol 16: 349-352 (1997) |
| | 5194 | |

TABLE 10
Polymorphisms in Collagen COL1A2
http://www.le.ac.uk/ge/collagen/col1a2.html#Table_4

| Polymorphism | Nucleotide | Reference |
|---|---|---|
| Thr29Thr ACT-ACC | N-propeptide 226 | Zhuang et al. Hum Mutat 7: 89-99 (1996) |
| Pro59Thr CCA-ACA | N-propeptide 314 | Kuivaniemi et al. Biochem J 252: 633-640 (1988) |
| Asp82Asp GAT-GAC | N-propeptide 385 | Strobel et al. Matrix 12 87-91 (1992) |
| Gly127Gly GGG-GGT | 790 | Filie et al. Hum Mutat 2: 380-388 (1993) |
| Gly139Gly GGT-GGC | 826 | Filie et al. Hum Mutat 2: 380-388 (1993) |
| Gly145Gly GGA-GGC | 844 | Filie et al. Hum Mutat 2: 380-388 (1993) |
| Val153Val GTA-GTG | 868 | Filie et al. Hum Mutat 2: 380-388 (1993) |
| Pro158Pro CCT-CCC | 883 | Filie et al. Hum Mutat 2: 380-388 (1993) |
| Asn159Ile AAT-ATT | 885 | Filie et al. Hum Mutat 2: 380-388 (1993) |
| Gly166Gly GGT-GGC | 907 | Filie et al. Hum Mutat 2: 380-388 (1993) |
| Gly172Gly GGT-GGC | 925 | Filie et al. Hum Mutat 2: 380-388 (1993) |
| Thr186Ala ACT-GCT | 965 | Filie et al. Hum Mutat 2: 380-388 (1993) |
| Gly187Gly GGA-GGT | 970 | Filie et al. Hum Mutat 2: 380-388 (1993) |
| Ser275Ser TCT-TCC | 1234 | Filie et al. Hum Mutat 2: 380-388 (1993) |
| Gly277Gly GGT-GGG | 1240 | Filie et al. Hum Mutat 2: 380-388 (1993) |
| Pro392Pro CCA-CCC | 1585 | Constantinou et al. Nucl Acids Res 18: 5577 (1990) |
| Val420Ala GTT-GCT | 1668 | Wenstrup et al. J Biol Chem 266: 2590-2594 (1991) |
| Ala459Pro GCT-CCT | 1784 | Bateman et al. Hum Mutat 1: 55-62 (1992) |
| Val536Val GTG-GTT | 2017 | Bateman et al. Am J Med Genet 45: 233-240 (1993) |
| T-G | +661 bp within IVS | Strobel et al. Matrix 12: 87-91 (1992) |
| | 33 | |
| Ala653Gly GCC-GGC | 2367 | Kuivaniemi et al. Biochem J 252: 633-640 (1988) |
| Arg732His CGT-CAT | 2604 | Zhuang et al. Hum Mutat 7: 89-99 (1996) |
| Pro795Pro CCT-CCA | 2791 | Baldwin et al. J Biol Chem 264: 3002-3006 (1989) |
| Gly862Gly GGC-GGT | 2995 | Baldwin et al. J Biol Chem 264: 3002-3006 (1989) |
| Phe932Leu TTC-TTA | 3205 | Baldwin et al. J Biol Chem 264: 3002-3006 (1989) |
| Gly955Gly GGC-GGT | 3274 | Strobel et al. Matrix 12: 87-91 (1992) |
| Thr983Thr ACG-ACA | 3358 | Baldwin et al. J Biol Chem 264: 3002-3006 (1989) |
| Leu1011Pro CTA-CCA | 3441 | Baldwin et al. J Biol Chem 264: 3002-3006 (1989) |
| Thr1058Pro ACC-CCC | 3581 | Oliver et al. Hum Mutat 7: 318-326 (1996) |
| Glu1099Asp GAA- | C-propeptide 3706 | Marini et al. J Biol Chem 268: 2667-2673 (1993) |
| GAT | | |
| Ala1100Ala GCC-GCT | C-propeptide 3709 | Marini et al. J Biol Chem 268: 2667-2673 (1993) |
| Cys1105Cys TGC-TGT | C-propeptide 3724 | Marini et al. J Biol Chem 268: 2667-2673 (1993) |
| Pro1108Ser CCT-TCT | C-propeptide 3731 | Makela et al. Biochim Biophys Acta 1049: 171-176 |
| | | (1990) |
| Val1152Val GTT-GTA | C-propeptide 3865 | Marini et al. J Biol Chem 268: 2667-2673 (1993) |
| Gly1194Gly GGC- | C-propeptide 3991 | Marini et al. J Biol Chem 268: 2667-2673 (1993) |
| GGA | | |

TABLE 11
Polymorphisms in ApoE nucleotide numbering according
to accession number K00396

| Nucleotide | Base change | Reference |
|---|---|---|
| 267 | g-c | J. Biol. Chem. 258, 11422-11422 (1983) |
| 279 | g-a | J. Biol. Chem. 258, 11422-11422 (1983) |
| 288 | g-a | J. Biol. Chem. 258, 11422-11422 (1983) |
| 409 | g-a | J. Biol. Chem. 259 6498-6504 (1984) |
| 586 | c in ε-3 allele; | Biochem. Biophys. Res. Commun. 130, |
| | t in ε-2 allele | 1261-1266 (1985); J. Biol. Chem. 259, |
| | | 5495-5499 (1984) |
| 702 | c-g | J. Biol. Chem. 258, 11422-11422 (1983) |
| 783 | c-t | www.ncbi.nlm.nih.gov:80/entrez/ |
| | (creating an NUX | query.fcgi?cmd=Retrieve&list_uids= |
| | NUX cleavage | 178850&dopt=GenBank |
| | site) | |
| 831 | g-t | M10065 |
| 858 | g-a | J. Biol. Chem. 258, 11422-11422 (1983) |

ApoE: http://srs.ebi.ac.uk/srs6bin/cgi-binlwgetz?−id+4Flds1EEB6c+−e+[HGBASE-GeneSymbol:apoe] and
http://www.ncbi.nim.nih.gov:80/entrez/query.fcgi?cmd=Retrieve&db=Nucleotide&list_uids=178850&dopt=Gen Bank

### Example 7

### Procedure for Genotyping OI Patients for the Presence of the 3210 Polymorphism

In order to determine whether cells from an OI patient may be used for treatment according to the methods of the invention, the patient is first genotyped to determine the presence of one or more polymorphisms and the disease causing mutation in a target gene and the disease-causing mutation. An exemplary procedure for determining the patient's suitability for genetic therapy using RzPolCol11 (targeting the 3210 T polymorphic variant of the COL1A1 transcript) is as follows.

A blood sample (e.g., 5 ml) is taken from the OI patient and centrifuged at 3000 rpm for 10 minutes and the pelleted cells treated with a standard lysis solution such as 1× tris/triton (10 mMTrisHCl pH9, 1% triton X-100) for 5 minutes and heated to 94° C. for 10 minutes to release DNA from the cells. PCR reactions are carried out on DNAs using standard protocols. Primers are designed to amplify exon 52 plus the 3′UTR across the polymorphic site. The forward primer is shown in SEQ ID NO: 10 (position 2977-2996 of accession number KO1228 and SEQ ID NO:20). The reverse primer is 5′GGTAAGGTTGAATGCACTTTTG 3′ (position 3326-3347 of accession number KO₁₂₂₈ and SEQ ID NO: 11). Resulting PCR products are cleaned using standard protocols and sequenced on an automated sequencer such as the AIB373A DNA sequencer, using a sequencing kit (e.g., Applied Biosystems) and the above reverse primer to assess the patient's genotype at position 3210 of Col1 A1. There are three possible genotypes: 3210 C/C which is an unsuitable genotype for gene therapy using RzPolCl1A1; 3210 T/T, which is ideal for cell treatment using RzPolCol1A1; and 3210 C/T, which must be further analyzed to determine whether or not the T-allele is on the same DNA strand as the mutation; only then is the patient suitable for gene therapy using RzPolCol1A1.

Skin biopsies (obtained by scraping skin or cheek swab) are taken from OI patients who are heterozygous for the 3210 polymorphism in Col1A1. RNA is extracted from biopsies using kits such as the PolyATract system 1000 (Promega) according to manufacturer's protocols. Complementary DNAs are synthesized from RNA using kits such as the Universal RiboClone cDNA synthesis system (Promega) and manufacturer's protocols. A long range PCR reaction is carried out on cDNAs from heterozygous C/T patients. Both the forward and reverse primer are typically 20 nucleotides long and have 50-60% G/C content. The forward primer binds within the 5′UTR of the COL1A1 gene and the reverse primer binds within the 3′UTR, such that the amplification product contains the entire coding sequence of the COL1A1 gene. In addition the reverse primer is designed so that its most 3′ nucleotide is exactly at the polymorphic 3210 site. The 3′ nucleotide is an adenine (A). In this way the reverse primer is specific and will only amplify DNA which has a T at position 3210. Thus most of the amplification product is taken from the T allele and a minority from the C allele. Over rounds of PCR amplification enrichment for the T-allele occurs. The longrange PCR reaction is carried out using a kit such as Expand Long Template PCR Systems (Boerhinger Mannheim), according to manufacturer's protocols. In addition, a negative control for the allele specific long range PCR will be carried out on cDNA derived from a person known to be homozygous C/C at position 3210. No amplification product should arise. Subsequent PCR product is sequenced across the entire COL1A1 gene using a series of primers (typically 20-mers with 50-60% G/C content) and an automated sequencing system as mentioned above. If a mutation is indeed found on the T-allele, cells of this heterozygous patient are suitable for treatment with RzPolCol1A1.

### Example 8

### MPC Gene Therapy

MPCs differentiate into, amongst others, osteoblasts, which are the target cells for OI. To undertake ex vivo gene therapy for OI, MPCs are taken from bone marrow of OI patients and transfected stably ex vivo with, for example, retroviruses carrying the therapeutic ribozyme and a replacement collagen gene with a non-suppressible polymorphism. MPCs which express both therapeutic ribozyme and replacement collagen gene are then selected, expanded ex vivo and subsequently transplanted back into OI patients.

Human MPC cells are obtained from mammalian subjects according to standard methods in the art, for example, those provided in Conget and Minguell (2000) or Allay et al. (1997). Briefly, 10 mls of bone marrow is obtained from the posterior iliac crest of adults who have given informed consent under e.g., an IRB-approved protocol to the Hematopoietic Stem Cell Facility of the Case Western Reserve University Ireland Cancer Center. Mononuclear cells are separated by centrifugation in a Ficoll-Hypaque gradient (density=1.077 g/cm³; Sigma), suspended in α-MEM containing 20% fetal bovine serum (FBS; Hyclone) and seeded at a concentration of 1×10⁶ cells/cm². Alternatively, single cell suspensions of bone marrow are layered on 70% (Sigma) gradients and low density mononuclear cells are recovered. 50×10⁶ cells are plated onto 100 mm² plastic tissue culture plates in DMEM+10% FBS, prescreened for growth and maintenance of the osteogenic potential of human MPCs as described in Goshima et al. 1991a,b; Lennon et al. (1995); and Allay et al. 1997. Cultures are maintained at 37° C. in a humidified atmosphere containing 5% CO₂. After 3 days, the medium is changed to remove nonadherent hematopoietic cells. Thereafter, the medium is changed twice weekly. Approximately 10-12 days after primary culture, the cells are detached from the plate with 0.25% trypsin containing 1 mM EDTA (GIBCO) for 5 min. at 37° C. The cells are diluted 1:3 and cyclically replated in fresh medium when cells reach 80% confluence. Methods and reagents for transducing mesenchymal stems cells are provided in the art, for example in U.S. Pat. Nos. 5,591,625; 5,486,359; 5,827,735; 5,226,914, the entire contents of which are incorporated herein by reference.

Human MPC cells are transfected according to standard methods in the art, for example, those provided in Allay et al. (1997). Briefly, MPC cells are obtained as described above. Human MPCs are grown in DMEM+20-30% heat-inactivated (HI) FBS for 18-24 hours following first or second passage to increase cell proliferation and enhance the rate of gene transfer. Medium is replaced with 4 mls of 0.45 mm-filtered viral supernatant (containing, 0.1-1000 MOI of retrovirus, virus comprising a desired ribozyme sequence), containing 6 mg/ml Polybrene (Sigma). After 6 hours, viral supernatant is removed and cells are cultured in DMEM+20-30% HI FBS for 18 hours and repeated daily for 4 days. Cultures of transduced human MPC are trypsinized and replated at clonal density in G418 to determine the number of clonal cells expressing the proviral genes, or expanded in G418. For all in vivo experiments, transduced cell populations, not individual clones, are generally used.

For X-Gal staining, an aliquot of virally transduced or untransduced human MPC are fixed in freshly prepared 2% formaldehyde, 0.2% glutaraldehyde in phosphate-buffered saline (PBS) for 5 minutes at 4° C., washed and stained in fresh 1 mg/ml X-Gal in 20 mM potassium ferrocyanide, 20 mM potassium ferricyanide, and 2 mM MgCl₂ in PBS and counterstained with 0.1% crystal violet.

Human MPC cells are transplanted according to standard methods in the art, for example, those provided in Horwitz et al. (1999). Briefly, approximately 5.7−7.5×10⁸ MPC cells/kg are infused into a patient by intravenous infusion. Moderate dose total body irradiation may be added to the regime for a patient if there is a mismatch at the HLA-DRβ1 allele. Cells are administered with cyclosporin (e.g., 45-60 mg/kg×2), phenyloin and bisulfan (e.g., 1 mg/kg×16) where appropriate or injected into bone. Notably chemoprophylaxis against graft versus host disease may not be required, as the MPCs are derived from the patient's own marrow. However, if required, chemoprophylaxis against graft versus host disease consists of intravenous cyclosporine 2.5 mg/kg every 12 hours), begun 2 days before transplantation. Patients receive 3 day courses of tetracycline at approximately 3 weeks and 1 week before biopsy. A 5.0-mm core of illiac bone is taken before and 6 months after transplantation with a trephine inserted through a 1.5 cm incision, from patients sedated by general anesthesia. Histological changes are determined on sections 5 μm in thickness of polymethyl methacrylate-embedded samples, using a Zeiss microscope.

### REFERENCES

Akai, J et al. (1999) Gene 239:65-73.
Antoniv, T T et al., (2001) J. Biol. Chem. 276(24):21754-64.
Ayre, B G et al. (1999) Proc. Natl. Acad. Sci. 96(7):3507-12.
Allay, J A et al. (1997) Hum. Gene Ther. 8(12):1417-27.
Bahramian, M B et al. (1999) Mol. Cell. Biol. 19(1):274-283.
Barabas, A P (1996) Br. Med. J. 5515:682-4.
Been and Cech, 1986, Cell, 47:207-216.
Bemoist and Chambon (1981) Nature 290:304-310.
Birikh, K R et al. (1997) Eur. J. Biochem. 245(1):1-16.
Borg, E et al. (2000) Acta Otolaryngol 120 (1):51-7.
Breault, et al. (1997) J. Biol. Chem. 272(50):31241-50.
Brinster, et al. (1982) Nature 296:39-42.
Buttini, M et al. (2000) Neuroscience 97(2):207-10.
Byers, P H (1989) Am. J. Med. Genet. 34(1):72-80.
Byers, P H (1990) Trends Genet. 6(9):293-300.
Byers, P H (1992) Second international symposium on the Marfan syndrome, Nov. 7-9, 1992 San Francisco, Calif. Hum. Mutat. 1993; 2(2):80-1.
Byers, P H et al. (1997) Am. J. Med. Genet. 72(1):94-105.
Carter, G and Lemoine, N R (1993) Cancer Res. 67: 869-876.
Cazenave et al. (1989) Nucleic Acids Res. 17: 4255-4273.
Campbell, T B et al. (1997) Nucleic Acids Res. 25(24):4985-93.
Chadwick, D R et al. (2000) Gene Ther. 7(16):1362-1368.
Chan, P P et al. (1997) J. Mol. Med. 75(4):267-282.
Chang, G Q et al. (1993) Neuron 11(4):595-605.
Chen, C J et al. (1992) Nucleic Acids Res. 20(17)4581-4589.
Chen, S J et al. (1999) J Invest Dermatol. 1112(1):49-57.
Chinnery, P F et al. (1999) Gene Ther. 6(12):1919-1928.
Chowrira, B M et al. (1994) J. Biol. Chem. 269(41):25856-64.
Conget, P A, and Minguell, J J. (2000) Exp. Hemat. 28:382-390.
Cornish-Bowden, A. and Wharton, C. W. Simple enzyme kinetics. In Enzyme Kinetics. D Rickwood, E D 1990 IRL Press, Oxford, pp. 14-45.
Courvalin, P et al. (1995) CR Acad. Sci. III 318(12):1207-12.
D'Alessio, M et al. (1991) Am. J. Hum. Genet. 49: 400-406.
Dalgleish, R et al. (1986) Hum. Genet. 73: 91-92.
Davidson, F F and Steller, H (1998) Nature 391(6667):587-91.
Dosaka-Akita, H et al. (1995) Cancer Res. 55:1559-1564.
Drenser, K A et al. (1998) Invest. Opthalmol. Vis. Sci. 39(5):681-9.
Dryja, T P et al. (1990) Nature 343: 364-366.
Duval-Valentin et al. (1992) Proc. Natl. Acad. Sci. USA 89: 504-508.
Eckstein, F (1985) Ann. Rev. Biochem. 54:367-402.
Ellis and Rodgers (1993) Nucleic Acids Res. 21: 5171-5178.
Farrar, G J et al. (1991) Nature 354: 478-480.
Farrar, G J et al. (1991) Genomics 14: 805-807.
Farrar, G J et al. (1995) Invest. Ophthamol. Vis. Sci. (ARVO) 36: (4).
Fedor, M J and Uhlenbeck, O C (1992) Biochemistry 31(48):12042-54.
Feldstein, P A et al. (1989) Gene 82(1):53-61.
Feng, M et al. (1995) Cancer Res. 55: 2024-2028.
Filie et al. (1993) Hum. Mut. 2: 380-388.
Fire, A et al. (1998) Nature 391(6669):806-811.
Furuichi, Y et al. (1977) Nature 266(5599):235-9.
Gaughan, D J et al. (1995) FEBS Letters 374: 241-245.
Gautier, et al. (1987) Nucl. Acids Res. 15:6625-6641.
Gaur, R K and Krupp G (1997) Methods Mol. Biol. 74:99-110.
Goshima, J et al. (1991a) Clin. Orthop. 262:298-311.
Goshima, J et al. (1991b) Biomaterials 12(2):253-8.
Hampson, G et al. (1998) Diabetologia 41(11):1314-20.
Hanvey, J C et al. (1992) Science 258: 1481-1485.
Hardenbol, P and Van Dyke, M W (1996) Proc. Natl. Acad. Sci. USA 93: 2811-2816.
Haseloff and Gerlach, 1988, Nature, 334:585-591
Haseloff, J et al. (1989) Gene 82(1):43-52.
Heidenreich, O and Eckstein, F (1992) J. Biol. Chem. 267(3):1904-9.
Helene, C (1991) Anticancer Drug Des. 6(6):569-584.
Helene, C et al. (1992) Ann NY Acad. Sci. 660:27-36.
Hendry, P et al. (1995) Nucleic Acids Res. 23(19):3922-7.
Hendry, P et al. (1997) In Ribozyme Protocols PC Turner ED Humana Press, New Jersey, US.
Herschlag, D et al. (1994) EMBO 13(12):2913-2924.
Herskowitz et al. (1987) Nature 329: 219-222.
Hertel, K J et al. (1994) Biochemistry 33(11):3374-85.
Horster, A et al. (1999) Gene Ther. 6(7):1231-1238.
Horwitz, E M et al. (1999) Nature Med. 5(3):309-13.
Humphries, P et al. (1992) Science 256: 804-808.
Humphries, M et al. (1997) Nature Genet. 15: 216-219.
Ianov, I P et al. (2000) Nucleic Acids Res. 28(17):3185-3196.
Inoue et al., 1987, Nucl. Acids Res. 15:6131-6148.
Inoue et al., 1987, FEBS Lett. 215:327-330.
Jankowsky, E and Schwenzer, B (1996) Biochemistry 35(48):15313-21.
Jankowsky, E and Schwenzer, B (1996) Nucleic Acids Res. 24(3):423-429.
Jones, J T et al. (1996) Nature Med. 2: 643-648.
Jordan, S A et al. (1993) Nature Genet. 4: 54-58.
Kajiwara, K et al. (1991) Nature 354: 480-483.
Kajiwara, K et al. (1994) Science 264(5165):1604-1608.
Keen, R W et al. (1999) Arthritis Rheum. 42(2):285-90.
Knudsen, H and Nielsen, P E (1996) Nucleic Acids Res. 24(3):494-500.
Krebsbach, P H et al. (1993) Mol. Cell. Biol. 13(9):5168-5174.
Krol et al., (1988) BioTechniques 6:958-976.
Kuwabara, T et al. (1996) Nucleic Acids Res. 24(12):2302-2310.
Kuwabara, T et al. (1998) Mol. Cell. 2(5):617-27.
Lange, W et al. (1993) Leukemia 7: 1786-1794.
Latchman, D S (2000) Histol. Histopathol. 15(4):1253-1259.
Lehmann, H W et al. (1994) Arch. Dermatol. Res. 286(8):425-8.
Leirdal, M and Sioud, M (1998) Biochem. Biophys. Res. Commun. 250 (1):171-4.
Lemaitre et al., 1987, Proc. Natl. Acad. Sci. 84:648-652.
Lennon, D P et al. (1995) Exp. Cell Res. 219(1):211-22.
Letsinger, R L et al., 1989, Proc. Natl. Acad. Sci. U.S.A. 86:6553-56.
Lewin, A S et al. (1998) Nature Med. 4(8):967-71.
Li, S W et al. (1998) Matrix Biol. 17(3):213-221.
Ludwig, J et al. (1998) Nucleic Acids Res. 26(10):2279-85.
Macejak, D G et al. (1999) J. Virol. 73(9):7745-7751.
Maher, L J et al. (1992) Bioassays 14(12):807-815.
Maheshwari, A et al. (2000) Mol. Ther. 2(2):121-130.
Mansergh, F et al. (1999) Am. J. Hum. Genet. 64(4):971-85.
Mansergh, F V et al. (1995) J. Med. Genet. 32: 855-858.
Mashhour, B et al. (1994) Gene Therapy 1: 122-126.
Matsumoto, T et al. (1994) Jpn. J. Hum. Genet. 39(1):205-206.
McCarron, M O and Nicoll, J A (2000) Ann. NY Acad. Sci. 903:179-9.
McKay, R A et al. (1996) Nucleic Acids Res. 24(3):411-417.
McNally et al. (1999) Hum. Mol. Genet. 8(7):1309-1312.
McWilliam, P et al. (1989) Genomics 5:612-619.
Millington-Ward, S et al. (1997) Hum. Mol. Genet. 6(9):1415-26.
Millington-Ward, S et al. (1999) Antisense Nucleic Acid Drug Dev. 9(6);537-42.
Miyatake, S et al. (1996) Jpn. J. Hum. Genet. 41(2):253-255.
Muramatsu, T et al. (2000) Int. J. Mol. Med. 7(1):61-66.
Nielsen, P E (2000) Pharmacol. Toxicol. 86(1):3-7.
Ohkawa, J et al. (1993) Proc. Natl. Acad. Sci. 9 USA 0:11302-11306.
Ohta, Y et al. (1996) Nucleic Acids Res. 24(5):938-942.
Ott, J et al. (1989) Proc. Natl. Acad. Sci. USA 87:701-704.
Oyama, T et al. (1995) Pathol. Int. 45: 45-50.
Phillips C L et al. (1990) J. Clin. Invest. 86: 1723-1728.
Pieken, W A et al. (1991) Science 253(5017):314-7.
Pieken, W A et al. (1991b) Nucleic Acids Symp. Ser. 1991b; (24):51-3.
Pittenger, M F et al. (1999) Science 284(5411):143-7.
Postel et al. (1991) Proc. Natl. Acad. Sci. USA 88: 8227-8231.
Porumb, H et al. (1996) Cancer Res. 56: 515-522.
Puttaraju, M et al. (1999) Nat. Biotechnol. 17(3):246-252.
Quattrone, A et al. (1995) Cancer Res. 55:90-95.
Reaves, P Y et al. (2000) Methods 22(3):211-218.
Redeker, E et al. (1993) Hum. Mol. Genet. 2(6):823.
Rimsky et al. (1989) Nature 341: 453-456.
Rossi, G A et al. (1983) Boll. Soc. Ital. Biol. Sper. 59(6):806-812.
Rupert et al. (1995) Eur. J. Hum. Genet. 3(6):333-343.
Sarin et al. (1988) Proc. Natl. Acad. Sci. USA 85:7448-7451.
Sarver et al., 1990, Science 247:1222-1225.
Saville, B J et al. (1990) Cell 61(4)685:696.
Sillence, D O et al. (1979) J. Med. Genet. 16(2):101-16.
Sioud, M et al. (1998) Nature Biotechnol. 16(6):556-561
Sizemore, D R et al. (1995) Science 270 (5234):299-302.
Sokolov, B P et al. (1993) Biochemistry 32(35):9242-9249.
Sokolov, B P et al. (1995) J. Biol. Chem. 270 (16):9622-9629.
Stage-Zimmerman, T K and Uhlenback, O C (1998) RNA 4(8):875-89.
Stein et al. (1988) Nucl. Acids Res. 16:3209.
Stein et al., (1988) Cancer Res 48:2659-2668.
Strayer, D S et al. (2000) Gene Ther. 7(10):886-895.
Sullenger, B A and Cech, T R. (1994) Nature 371:619-622.
Sun, J S et al. (1989) Proc. Natl. Acad. Sci. USA 86:9198-9202.
Tam, P et al. (2000) Gene Ther. 7(21): 1867-1874.
Takahashi, M et al. (1999) J. Virol. 73(9):7812-7816.
Taylor, R W et al. (1997) Nature Genet. 15:212-215.
Trauger, J W et al. (1996) Nature 382:559-561.
Valera, A et al. (1994) J. Biol. Chem. 269:28543-28546.
Van der Krol et al., (1988) Biotechniques 6:958-976.
Van Soest, S et al. (1994) Genomics 22:499-504.
Van Stijn, T et al. (1995) Anim. Genet. 26(4):279.
Vasan, N S et al. (1991) Am. J. Hum. Genet. 48:305-317.
Wagner et al. (1981) Proc. Natl. Acad. Sci. U.S.A. 78:1441-1445.
Wagner, R. 1994. Nature 372:333.
Wang, F S et al. (1999) Int. J. Cancer 81(6):944-950.
Wei, Z et al. (1996) Nucleic Acids Res. 24:(4) 655-661.
Wenstrup, R J et al. (1990) Am. J. Hum. Genet. 46(5):975-92.
Westerhausen, A I et al. (1990) Nucleic Acids Res. 18:4968.
Wevrick, R et al. (1994) Hum. Mol. Genet. 3(10):1877-1882.
Willing, M C et al. (1993) Am. J. Hum. Genet. 45:223-227.
Wu, H N et al. (1989) Proc. Natl. Acad. Sci. USA 86(6):1831-1835.
Yamamoto et al. (1980) Cell 22:787-797.
Yee, J K et al. (1994) Methods Cell. Biol. 43 Pt A:99-112.
Zack, D J et al. (1991) Neuron. 6(2):187-99.
Zack, D J et al. (1994) Vis. Neurosci. 11(6):1227-1231.
Zaug, A J et al. (1988) Biochemistry 27(25);8924-31.
Zaug, et al., 1984, Science, 224:574-578.
Zaug and Cech, 1986, Science, 231:470-475.
Zaug, et al., 1986, Nature, 324:429-433.
Zhang, et al. (1999) Biochemistry 38(37):12111-23.
Zhuang J et al. (1996) Hum. Mut. 7:89-99.
Zon (1988) Pharm. Res. 5:539-549.

### EQUIVALENTS

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes which come within the meaning and range of equivalency of the claims are therefore intended to be embraced herein.

1. A method for suppressing the expression of a mutant allele of a gene in a cell, the method comprising the steps of:
selecting a mutant allele which encodes a message comprising a nucleotide region comprising an NUX ribozyme cleavage site within or adjacent to a polymorphic variation characteristic of the mutant allele; and
introducing into the cell a ribozyme that hybridizes with the message within or adjacent to the polymorphic variation, wherein the ribozyme cleaves the message at the NUX ribozyme cleavage site.

2. The method of claim 1, wherein the ribozyme is operatively linked to an expression vector.

3. The method of claim 1, wherein the ribozyme is specific for mammalian collagen 1A1 RNA comprising a T3210C polymorphism, wherein the nucleotide at position 3210 is a T.

4. The method of claim 1, wherein the ribozyme is specific for mammalian collagen 1A2 RNA comprising an A902G polymorphism, wherein the nucleotide at position 902 is an A or T907A polymorphism, wherein the nucleotide at position 907 is a T.

5. The method of claim 1, wherein the ribozyme is specific for mammalian rhodopsin RNA comprising a polymorphism selected from the group consisting of Pro23Leu, Gly120Gly and Ala173Ala.

6. The method of claim 1, wherein the ribozyme is specific for mammalian peripherin RNA having a polymorphism selected from the group consisting of C558T, Glu304Gln, Lys310Arg and Gly338Asp.

7. The method of claim 1, further comprising the step of providing a replacement nucleic acid which is not cleaved by, or is only partially inhibited by, the ribozyme, the replacement nucleic acid comprising the nucleotide sequence for an allele of the gene which encodes a normal or non-disease-causing protein.

8. The method of claim 7, wherein the normal or non-disease-causing protein is selected from the group consisting of rhodopsin, collagen 1A1, collagen 1A2 and peripherin.

9. A suppression effector comprising a ribozyme that hybridizes on either side of a polymorphic variation of a nucleic acid, and wherein said ribozyme cleaves the nucleic acid with the polymorphic variation but does not cleave a nucleic acid that does not contain the polymorphic variation.

10-13. (canceled)

14. A method for suppressing the expression of a mutant allele of a gene, the method comprising the steps of
providing a suppression effector comprising a nucleic acid having a sequence complementary to a region of a mutant mRNA comprising a polymorphic variation, in an amount sufficient to suppress the expression of the mutant mRNA; and
providing a replacement nucleic acid comprising a nucleotide sequence encoding a non-disease-causing allele of the gene which does not comprise the polymorphic variation, and is not suppressed, or is only partially suppressed, by the suppression effector.

15. The method of claim 14, wherein the suppression effector is a nucleic acid or a peptide nucleic acid (PNA).

16. The method of claim 14, wherein the suppression effector is a peptide or antibody.

17. The method of claim 14, wherein the suppression effector is operatively linked to an expression vector.

18. The method of claim 15, wherein the nucleic acid is an antisense nucleic acid.

19. (canceled)

20. The method of claim 14, wherein the suppression effector is a ribozyme.

21. The method of claim 15, wherein the suppression effector is a nucleic acid that forms a triple helix with the allele comprising the polymorphic variation.

22. The method of claim 14, wherein the replacement nucleic acid is a wild-type or non-disease causing allele of the gene which does not comprise the polymorphism.

23. The method of claim 14, wherein the replacement nucleic acid is operatively linked to an expression vector.

24. The method of claim 14, wherein the polymorphic variation is located in one or more sites selected from the group consisting of a coding region, a 5′ untranslated region, a 3′ untranslated region and an intronic region.

25-26. (canceled)

27. A suppression effector complementary to a region of an mRNA comprising a polymorphic variation, wherein the suppression effector suppresses the expression of the mRNA comprising the polymorphic variation but does not inhibit, or only partially inhibits, the expression of an mRNA that does not comprise the polymorphic variation, the suppression effector comprising a ribozyme having a nucleic acid sequence that hybridizes to either side of the polymorphic variation.


SEQUENCE LISTING (34 sequences)

SEQ ID NO: 1 - DNA, 473 bp, Homo sapiens
  misc_feature: human rhodopsin cDNA
ggcttatcgaaattaatacgactcactatagggagacccaagcttagagtcatccagctggagccctgagtggctgagctcaggccttcgcagcattcttgggtgggagcagccacgggtcagccacaagggccacagccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaat

SEQ ID NO: 2 - DNA, 20 bp, Artificial Sequence
  5′ human rhodopsin primer over Hind III site
acccaagcttagagtcatcc

SEQ ID NO: 3 - DNA, 20 bp, Artificial Sequence
  3′ human rhodopsin primer over BstEII site
accatgcgggtgacctcctt

SEQ ID NO: 4 - DNA, 20 bp, Artificial Sequence
  5′ human rhodopsin primer over mutation site
atcgtgctggtcttccccat

SEQ ID NO: 5 - DNA, 20 bp, Artificial Sequence
  3′ human rhodopsin primer over mutation site
atggggaagaccagcacgat

SEQ ID NO: 6 - DNA, 19 bp, Artificial Sequence
  5′ human collagen 1A2 primer
cagagatggtgaagatggt

SEQ ID NO: 7 - DNA, 20 bp, Artificial Sequence
  3′ human collagen 1A2 primer
agtcctctggcaccagtagc

SEQ ID NO: 8 - DNA, 23 bp, Artificial Sequence
  5′ human collagen 1A2 mutagenesis primer
taacgctggtcctactggacccg

SEQ ID NO: 9 - DNA, 23 bp, Artificial Sequence
  3′ human collagen 1A2 mutagenesis primer
cgggtccagtaggaccagcgtta

SEQ ID NO: 10 - DNA, 20 bp, Artificial Sequence
  5′ human collagen 1A1 primer
agtcacaccggagcctgggg

SEQ ID NO: 11 - DNA, 22 bp, Artificial Sequence
  3′ human collagen 1A1 primer
ggtaaggttgaatgcacttttg

SEQ ID NO: 12 - DNA, 335 bp, Homo sapiens
  misc_feature: human rhodopsin cDNA with C to G change at position 477
acgggtcagccacaagggccacagccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtgaccgtccagcacaagaagctgcgcacgcctctcaactacatcctgctcaacctagccgtggctgacctcttcatggtcctaggtggcttcaccagcaccctctacacctctctgcatggatacttc

SEQ ID NO: 13 - DNA, 358 bp, Homo sapiens
  misc_feature: human rhodopsin with C to T change at codon 23
cagccacaagggccacagccatgaatggcacagaaggccctaacttctacgtgcccttctccaatgcgacgggtgtggtacgcagcctcttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttgga

SEQ ID NO: 14 - DNA, 37 bp, Artificial Sequence
  Rz10 hammerhead ribozyme
ggacggtctgatgagtccgtgaggacgaaacgtagag

SEQ ID NO: 15 - DNA, 37 bp, Artificial Sequence
  Rz20 hammerhead ribozyme
tactcgaactgatgagtccgtgaggacgaaaggctgc

SEQ ID NO: 16 - DNA, 3347 bp, Homo sapiens
  Unsure (1871)..(1871): Unknown
ggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccccgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggaccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgaccggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgccaaaggcgatgctggtccccctgggcctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccccggtcccatgggcccccctggattggctggaccccctggtgaatctggacgtgagggggctcctgctgccgaaggttcccctggacgagacggttctcctggcgccaagggtgaccgtggtgagaccggccccgctggaccccctggtgctcntggtgctcntggtgcccctggccccgttggccctgctggcaagagtggtgatcgtggtgagactggtcctgctggtcccgccggtcccgtcggccccgctggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctcccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcncccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctcnagggctccaacgagatcgagatccgcgccgagggcaacagccgcttcacctacagcgtcactgtcgatggctgcacgagtcacaccggagcctggggcaagacagtgattgaatacaaaaccaccaagtcctcccgcctgcccatcatcgatgtggcccccttggacgttggtgccccagaccaggaattcggcttcgacgttggccctgtctgcttcctgtaaactccctccatcccaacttggctccctcccacccaaccaactttccccccaacccggaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttacc

SEQ ID NO: 17 - DNA, 370 bp, Homo sapiens
  misc_feature: human collagen 1A1 cDNA Allele A
gtcacaccggagcctggggcaagacagtgattgaatacaaaaccaccaagtcctcccgcctgcccatcatcgatgtggcccccttggacgttggtgccccagaccaggaattcggcttcgacgttggccctgtctgcttcctgtaaactccctccatcccaacttggctccctcccacccaaccaactttccccccaacccggaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttacc

SEQ ID NO: 18 - DNA, 370 bp, Homo sapiens
  misc_feature: human collagen 1A1 cDNA Allele B
gtcacaccggagcctggggcaagacagtgattgaatacaaaaccaccaagtcctcccgcctgcccatcatcgatgtggcccccttggacgttggtgccccagaccaggaattcggcttcgacgttggccctgtctgcttcctgtaaactccctccatcccaacttggctccctcccacccaaccaactttccccccaacccggaacagacaagcaacccaaactgaacccccccaaaagccaaaaaatgggagacaatttcacatggactttggaaaatatttttttcctttgcattcatctctcaaacttagtttttatctttgaccaaccgaacatgaccaaaaaccaaaagtgcattcaaccttacc

SEQ ID NO: 19 - DNA, 37 bp, Artificial Sequence
  RzPolCol1A1 hammerhead ribozyme
tggcttttctgatgagtccgtgaggacgaaagggggt

SEQ ID NO: 20 - DNA, 2372 bp, Homo sapiens
  misc_feature: human type I collagen 1A2 cDNA
ggcgggccaggtgatacctccgccggtgacccaggggctctgcgacacaaggagtctgcatgtctaagtgctagacatgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtcccgatggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcatggtgctgtaggtgcccctggtcctgctggagccacaggtgaccggggcgaagctggggctgctggtcctgctggtcctgctggtcctcggggaagccctggtgaacgtggcgaggtcggtcctgctggccccaacggatttgctggtccggctggtgctgctggtcaaccgggtgctaaaggagaaagaggaggcaaagggcctaagggtgaaaacggtgttgttggtcccacaggccccgttggagctgctggcccagctg

SEQ ID NO: 21 - DNA, 879 bp, Homo sapiens
  misc_feature: human collagen 1A2 (A) cDNA with G at position 902 and T at position 907
cagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggact

SEQ ID NO: 22 - DNA, 879 bp, Homo sapiens
  misc_feature: human collagen 1A2 (B) cDNA with A at positions 902 and907
cagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctactggacccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggact

SEQ ID NO: 23 - DNA, 36 bp, Artificial Sequence
  Rz902 hammerhead ribozyme
ggtccagctgatgagtccgtgaggacgaaaggacca

SEQ ID NO: 24 - DNA, 35 bp, Artificial Sequence
  Rz907 hammerhead ribozyme
cggcggctgatgagtccgtgaggacgaaaccagca

SEQ ID NO: 25 - DNA, 17 bp, Artificial Sequence
  Collagen 1A1 forward primer
caggaattcggcttcga

SEQ ID NO: 26 - DNA, 20 bp, Artificial Sequence
  Collagen 1A1 reverse primer
ggttcagtttgggttgcttg

SEQ ID NO: 27 - DNA, 20 bp, Artificial Sequence
  Collagen 1A2 forward primer
caaggatgcactatggatgc

SEQ ID NO: 28 - DNA, 20 bp, Artificial Sequence
  Collagen 1A2 reverse primer
ggagctcctataccagttct

SEQ ID NO: 29 - DNA, 20 bp, Artificial Sequence
  GAPDH forward primer
cagcctcaagatcatcagca

SEQ ID NO: 30 - DNA, 20 bp, Artificial Sequence
  GAPDH reverse primer
catgagtccttccacgatac

SEQ ID NO: 31 - DNA, 20 bp, Artificial Sequence
  NeoF RT PCR primer
gatgcctgcttgccgaatat

SEQ ID NO: 32 - DNA, 20 bp, Artificial Sequence
  NeoR RT PCR primer
gctcttcagcaatatcacgg

SEQ ID NO: 33 - DNA, 20 bp, Artificial Sequence
  pLRNLP RT PCR primer
aagacaggatatcagtggtc

SEQ ID NO: 34 - DNA, 20 bp, Artificial Sequence
  pLRNLR RT PCT primer
ctatggctcgtactctatag